SEQUENCE LISTING (40 sequences)

SEQ ID NO: 1 - DNA, 23 bp, Artificial Sequence
  synthetic oligonucleotide
  misc_feature (1)..(23)
acaatccgggcaatctccattgg

SEQ ID NO: 2 - protein, 1368 aa, Streptococcus pyogenes [sequence omitted]

SEQ ID NO: 3 - protein, 1394 aa, Artificial Sequence
  synthetic polypeptide
  MISC_FEATURE (1)..(1394): Cas9 from S. pyogenes with linker between Cas9 and HIS tag
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVAAACAAALEHHHHHH

SEQ ID NO: 4 - protein, 1368 aa, Artificial Sequence
  synthetic polypeptide
  MISC_FEATURE (1)..(1368): dCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1307 aa, Artificial Sequence
  synthetic polypeptide
  MISC_FEATURE (1)..(1307): ddCasCfp1
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 6 - protein, 1228 aa, Artificial Sequence
  synthetic polypeptide
  MISC_FEATURE (1)..(1228): ddLbCfp1
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 7 - protein, 1314 aa, Artificial Sequence
  synthetic polypeptide
  MISC_FEATURE (1)..(1314): ddFnCfp1
MYPYDVPDYASGSGMSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 8 - DNA, 60 bp, Artificial Sequence
  synthetic oligonucleotide
  misc_feature (1)..(60): wild type
atagagcaggtatcttgagaagccaatggagattgcccggattgtggcccggtgcctgtg

SEQ ID NO: 9 - DNA, 57 bp, Artificial Sequence
  synthetic oligonucleotide
  misc_feature (1)..(57): Mutation 1
atagagcaggtatcttgagaagccaatgggaattcccccgttttgccggtgcctgtg

SEQ ID NO: 10 - DNA, 56 bp, Artificial Sequence
  synthetic oligonucleotide
  misc_feature (1)..(56): Mutation 2
atagagcaggtatcttgagaagccaagggaattcccgttgtggcccggtgcctgtg

SEQ ID NO: 11 - DNA, 59 bp, Artificial Sequence
  synthetic oligonucleotide
  misc_feature (1)..(59): Mutation 3
atagagcaggtatcttgagaagccaagggaaattcccccgatttggcccggtgcctgtg

SEQ ID NO: 12 - DNA, 54 bp, Artificial Sequence
  synthetic oligonucleotide
  misc_feature (1)..(54): Mutation 4
atagagcaggtatcttgagaagccaaggaattcccccatttcccggtgcctgtg

SEQ ID NO: 13 - DNA, 59 bp, Artificial Sequence
  synthetic oligonucleotide
  misc_feature (1)..(59): Mutation 5
atagagcaggtatcttgagaagccaatggaattcccggatttgtggcccggtgcctgtg

SEQ ID NO: 14 - DNA, 61 bp, Artificial Sequence
  synthetic oligonucleotide
  misc_feature (1)..(61): Mutation 6
atagagcaggtatcttgagaagccaatggagaattgcccggattgtggcccggtgcctgtg

SEQ ID NO: 15 - DNA, 61 bp, Artificial Sequence
  synthetic oligonucleotide
  misc_feature (1)..(61): Mutation 7
atagagcaggtatcttgagaagccaatggagattgccccggattgtggcccggtgcctgtg

SEQ ID NO: 16 - DNA, 60 bp, Artificial Sequence
  synthetic oligonucleotide
  misc_feature (1)..(60): Mutation 8
atagagcaggtatcttgagaagccaatggagattgccccggatttggcccggtgcctgtg

SEQ ID NO: 17 - DNA, 660 bp, Artificial Sequence
  synthetic oligonucleotide
  misc_feature (1)..(660): Cas9-C construct nucleic acid (entire sequence)
gactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggcggccgcactcgagcaccaccaccaccaccacttgagatccggctgctaacaaagcccg

SEQ ID NO: 18 - protein, 216 aa, Artificial Sequence
  synthetic polypeptide
  MISC_FEATURE (1)..(216): Cas9-C construct protein sequence (entire sequence)
DFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVAAALEHHHHHHLRSGC

SEQ ID NO: 19 - protein, 7 aa, Artificial Sequence
  synthetic polypeptide
  MISC_FEATURE (1)..(7): SV40 NLS
PKKKRKV

SEQ ID NO: 20 - protein, 6 aa, Artificial Sequence
  synthetic polypeptide
  MISC_FEATURE (1)..(6): His Tag
HHHHHH

SEQ ID NO: 21 - DNA, 660 bp, Artificial Sequence
  synthetic oligonucleotide
  misc_feature (1)..(660): Cas9-5C construct nucleic acid (entire sequence)
ctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggcggccgcatgttgctgttgctgtgcggccgcactcgagcaccaccaccaccaccactgagatccg

SEQ ID NO: 22 - protein, 217 aa, Artificial Sequence
  synthetic polypeptide
  MISC_FEATURE (1)..(217): Cas9-5C construct protein sequence (entire sequence)
LEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVAAACCCCCAAALEHHHHHH

SEQ ID NO: 23 - protein, 5 aa, Artificial Sequence
  synthetic polypeptide
  MISC_FEATURE (1)..(5): Cys Tag
CCCCC

SEQ ID NO: 24 - DNA, 600 bp, Artificial Sequence
  synthetic orligonucleotide
  misc_feature (1)..(600): Cas9-C only nucleic acid
gactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtg

SEQ ID NO: 25 - protein, 189 aa, Artificial Sequence
  synthetic polypeptide
  MISC_FEATURE (1)..(189): Cas9-C only protein acid
DFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - DNA, 600 bp, Artificial Sequence
  synthetic oligonucleotide
  misc_feature (1)..(600): Cas9-5C only nucleic acid
ctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggcggcc

SEQ ID NO: 27 - protein, 187 aa, Artificial Sequence
  synthetic polypeptide
  MISC_FEATURE (1)..(187): Cas9-5C only protein acid
LEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 28 - DNA, 20 bp, Artificial Sequence
  synthetic oligonucleotide
  misc_feature (1)..(20): Sense phosphorothioate oligonucleotide
tccatgagcttcctgatgct

SEQ ID NO: 29 - DNA, 20 bp, Artificial Sequence
  synthetic oligonucleotide
  misc_feature (1)..(20): antisense phosphorothioate oligonucleotide
agcatcaggaagctcatgga

SEQ ID NO: 30 - DNA, 23 bp, Artificial Sequence
  synthetic oligonucleotide
  misc_feature (1)..(23): gRNA for PS-Cas9-STAT3 RNP
acaatccgggcaatctccattgg

SEQ ID NO: 31 - DNA, 23 bp, Artificial Sequence
  synthetic oligonucleotide
  misc_feature (1)..(23): gRNA for PS-Cas9-PDCD1 RNP
catgtggaagtcacgcccgttgg

SEQ ID NO: 32 - DNA, 23 bp, Artificial Sequence
  synthetic oligonucleotide
  misc_feature (1)..(23): gRNA for PS-Cas9-PDCD2 RNP
ccccttcggtcaccacgagcagg

SEQ ID NO: 33 - DNA, 23 bp, Artificial Sequence
  synthetic oligonucleotide
  misc_feature (1)..(23): gRNA for PS-Cas9-TET2 RNP
ggatagaaccaaccatgttgagg

SEQ ID NO: 34 - DNA, 23 bp, Artificial Sequence
  synthetic oligonucleotide
  misc_feature (1)..(23): gRNA for PS-Cas9-PARG RNP
accagttggatggacactaaagg

SEQ ID NO: 35 - DNA, 23 bp, Artificial Sequence
  synthetic oligonucleotide
  misc_feature (1)..(23): gRNA for PS-Cas9-5C-TCRA/TCRB RNP (TCRA)
agagtctctcagctggtacacgg

SEQ ID NO: 36 - DNA, 23 bp, Artificial Sequence
  synthetic oligonucleotide
  misc_feature (1)..(23): gRNA for PS-Cas9-5C-TCRA/TCRB RNP (TCRB)
gcagtatctggagtcattgaggg

SEQ ID NO: 37 - DNA, 23 bp, Artificial Sequence
  synthetic oligonucleotide
  misc_feature (1)..(23): gRNA for PS-S-Cas9-STAT3 RNP1
cattcgactcttgcaggaagcgg

SEQ ID NO: 38 - DNA, 23 bp, Artificial Sequence
  synthetic oligonucleotide
  misc_feature (1)..(23): gRNA for PS-S-Cas9-STAT3 RNP2
agtggcatgtgattctttgctgg

SEQ ID NO: 39 - DNA, 23 bp, Artificial Sequence
  synthetic oligonucleotide
  misc_feature (1)..(23): gRNA for PS-S-dCas9-VP64 RNPA (VEGFA-A)
gagcagcgtcttcgagagtgagg

SEQ ID NO: 40 - DNA, 23 bp, Artificial Sequence
  synthetic oligonucleotide
  misc_feature (1)..(23): gRNA for PS-S-dCas9-VP64 RNPB (VEGFA-B)
ggtgagtgagtgtgtgcgtgtgg

(19) United States
(12) Patent Application Publication
(10) Pub. No.: US 2024/0110176 A1
(43) Pub. Date: Apr. 4, 2024

(54) PHOSPHOROTHIOATE NUCLEIC ACID CONJUGATES INCLUDING DNA EDITING ENZYMES

(71) Applicant: CITY OF HOPE, Duarte, CA (US)

(72) Inventors: Hua YU, Duarte, CA (US); Yi-Jia LI, Duarte, CA (US); Andreas HERRMANN, Duarte, CA (US)

(21) Appl. No.: 17/999,680

(22) PCT Filed: May 21, 2021
(86) PCT No.: PCT/US21/33760

§ 371 Date: Nov. 22, 2022

Related U.S. Application Data
(60) Provisional application No. 63/029,225, filed on May 22, 2020.

(51) Int. Cl. C12N 15/11; C12N 9/22

(52) U.S. Cl. CPC C12N 15/11; C12N 9/22; C12N 2310/20; C12N 2310/315; C12N 2310/3513

(57) ABSTRACT

Provided herein are, inter alia, complexes useful for editing (e.g., repairing, modifying) DNA in a cell in vitro and in vivo. The complexes provided herein include a DNA editing agent bound to a phosphorothioate nucleic acid through a chemical linker. The chemical linker (e.g., disulfide linker) may be a linker that dissociates once the complex has entered the inside of the cell, thereby releasing the DNA editing agent and allowing the DNA editing agent to access and edit a cellular target sequence. The complexes provided herein exhibit high transfection efficiency and editing efficacy and therefore provide for useful therapeutic and diagnostic tools.

### CROSS-REFERENCES TO RELATED APPLICATIONS

This application claims the benefit of U.S. Provisional Application No. 63/029,225, filed May 22, 2020, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under NIH CA247368 awarded by The National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-764001WO_ST25.TXT, created on May 21, 2021, and having a size of 89,323 bytes, machine format IBM-PC, MS-Windows operating system, is hereby incorporated by reference.

### BACKGROUND OF THE INVENTION

Editing gene(s) in cells such as T cells, hematopoietic stem cells, cancer cells and other types of cells can have high impact on treating various diseases. As one example, Cas9-mediated gene deletion in CAR-T cells ex vivo results in improved efficacy of the CAR-T cells in cancer patients (Edward Stadtmauer et al, Science, 2020). However, current methodologies to introduce Cas9 enzyme with guide RNA(s) into cells, especially T cells and hematopoietic stem cells, face multiple challenges. Electroporation of Cas9 protein/guide RNA into T cells can only achieve low transfection efficiency while killing and weakening T cells. After electroporation of Cas9 protein/guide RNA, only a very small number of T cells can be transduced by lentiviral vectors (Edward Stadtmauer et al, Science, 2020), which is needed to generate CAR-T cells. Using lentivirus to deliver Cas9 encoding gene and specific guide RNA(s) also adds unnecessary viral genes into therapeutic T cells and causes manufacturing delays, reducing the chance to have successful CAR-T therapy for patients. Provided herein are, inter alia, solutions to these and other problems in the art.

### BRIEF SUMMARY OF THE INVENTION

In an aspect, a complex for delivering a gene editing agent to a cell is provided. The complex includes a gene editing agent covalently bound to a phosphorothioate nucleic acid through a chemical linker.

In an aspect, a complex for delivering a gene editing agent to a cell is provided. The complex includes (i) a double-stranded phosphorothioate oligonucleotide; (ii) a first gene editing agent covalently bound to a first phosphorothioate nucleic acid through a first chemical linker; and (ii) a second gene editing agent covalently bound to a second phosphorothioate nucleic acid through a second chemical linker; wherein at least a portion of the first phosphorothioate nucleic acid and a portion of the second phosphorothioate nucleic acid are complementary to each other and wherein at least a portion of the first phosphorothioate nucleic acid is hybridized to at least a portion of the second phosphorothioate nucleic acid thereby forming the double-stranded phosphorothioate oligonucleotide.

In another aspect, a complex for delivering a gene editing agent to a cell is provided. The complex includes (i) a double-stranded phosphorothioate oligonucleotide; (ii) a gene editing agent covalently bound to a first phosphorothioate nucleic acid through a first chemical linker; and (ii) a targeting agent covalently bound to a second phosphorothioate nucleic acid through a second chemical linker, wherein at least a portion of the first phosphorothioate nucleic acid and a portion of the second phosphorothioate nucleic acid are complementary to each other. And wherein at least a portion of the first phosphorothioate nucleic acid is hybridized to at least a portion of the second phosphorothioate nucleic acid thereby forming the double-stranded phosphorothioate oligonucleotide.

In an aspect, a pharmaceutical composition is provided. The pharmaceutical composition includes a pharmaceutical excipient and a complex as provided herein including embodiments thereof.

In an aspect, a method of delivering a gene editing agent to a cell is provided. The method includes, s contacting a cell with a complex as provided herein including embodiments thereof thereby delivering the gene editing agent to the cell.

### BRIEF DESCRIPTION OF THE DRAWINGS

FIG. 1 is a representative image of a gel showing phosphorothioated-oligo (PS) conjugation to Cas9 protein with primary amine-aldehyde covalent bond. Cas9 proteins were mixed with aldehyde-phosphorothioated-DNA oligos and incubated at 1 to 5 ratio and for the indicated time points. Phosphorothioated-DNA oligo was conjugated with FAM to label the modified Cas9 with green fluorescence (Abs λₘₐₓ=495 nm, Em λₘₐₓ=520 nm) to enable detection.

FIGS. 2A and 2B show representative images of gels of phosphorothioated-DNA oligo (PS-oligo) conjugated to Cas9-Cys protein with disulfide bonds. FIG. 2A. Cas9-Cys protein and thiol-phosphorothioated-oligo were incubated at 1 to 5 ratio and incubated for 16 hours. The thiol-phosphorothioated-oligo is conjugated to Cas9-Cys through disulfide bond formation. 15 Cas9-Cys-PS indicates the conjugation mixture without protein purification. 2^{nd} Cas9-Cys-PS presents Cas9-Cys-PS purified after PS conjugation. Left panel shows Coomassie blue staining and the right panel indicates the fluorescence labeled Cas9-Cys-PS. FIG. 2B. In vitro Cas9 activity assay. A STAT3 guild RNA-targeted DNA fragment (2.1 kb) was amplified by PCR and used as Cas9 substrate for in vitro enzymatic assay. As shown, the full-length DNA substrate was cleaved into two 1.5 and 0.6 kb DNA fragments by 200 ng of Cas9 after 2 hours incubation at 37° C. After PS conjugation and purification, same amount (200 ng) of unmodified or modified Cas9-Cys was used to perform in vitro enzymatic activity assay.

FIGS. 3A and 3B show efficient cellular entry of the modified Cas9/guide RNA using 1 or 5 PS-oligo per 1 Cas9/guide RNA.

FIGS. 4A and 4B show that the cell penetrating Cas9-Cys-PS protein shows great efficiency to internalize into mouse splenic T cells. (FIG. 4A) 2 μg of Cas9-Cys-PS was incubated with 1×10⁵ or 1×10⁶ of mouse splenic CD3+ T cells (with or without CD3 and CD28 activation) for 16 hours. Cell penetration of Cas9-Cys-PS was examined by flow cytometry. (FIG. 4B) The green fluorescent intensity was shown as indicated.

FIGS. 5A and 5B illustrate that the cell penetrating Cas9-Cys-PS protein efficiently penetrates into CAR-T cells. FIG. 5A. 5 μg of PS-modified protein was incubated with 1×10⁵ of mock or CAR-T cells for 16 hours. Cell penetration of PS-modified protein was examined by flow cytometry. Mock-T, human T cells without lentivirus mediated CAR-T engineering. CAR-T, human T cells that have been engineered through lentivirus transduction. Trypsin was added to eliminate cell surface binding of PS-modified protein. FIG. 5B. The green fluorescent intensity was shown as indicated.

FIG. 6 shows STAT3 gRNA-targeted DNA sequences used for Cas9-Cys-PS conjugates. The cell penetrating Cas9-Cys-PS induced 90% of STAT3 gene mutation in human T cells. Human T cells were incubated with Cas9-Cys-PS for 72 hours before the DNA was extracted. The STAT3 gRNA-targeted DNA fragment was amplified by PCR and subcloned to pCR2.1 DNA vector. The gene mutation frequency was examined by Sanger DNA sequencing. The DNA sequence highlighted in blue shows the PAM sequence and the nucleotides in yellow are the insertion mutations. The red hyphen signs indicate the deletion of nucleotides. “m”, nucleotide mutation. The frequency per type of DNA mutation is shown by percent.

FIGS. 7A-7F show the cell penetration and gene disruption of PS-Cas9-RNP in human CAR-T cells. FIG. 7A shows cell penetration efficiency of PS-Cas9-RNP labeled with FITC in human CD19 CAR-T cells was examined by fluorescent microscopy. Scale bar, 20 μm. FIG. 7B shows intracellular PS-Cas9-RNP was monitored by fluorescent gel imaging and western blotting after adding PS-Cas9-RNP to human CAR-T cells at the indicated concentrations. FIG. 7C shows the cell viability of CAR-T cells was not affected by PS-Cas9-RNP. 1×10⁵ CAR-T cells were cultured in absence or presence of PS-Cas9-RNP at the indicated concentrations for 4 days. FIGS. 7D, 7E and 7F. 10 μg (58.8 pmole) of PS-Cas9-RNP was added to 1×10⁵ CAR-T cells and cultured for 4 days. FIG. 7D shows the cell penetrating efficacy of PS-Cas9-PDCD1 RNP or PS-Cas9-TET2 RNP or both was examined by flow cytometry. The results indicated that PS-Cas9-RNP can penetrate more than 90% of CAR-T cells. FIG. 7E shows the gene perturbation of TET2 in CAR-T was examined by western blotting (left panel) and DNA sequencing of targeted TET2 loci (right panel) after PS-Cas9-TET2 RNP transfection. FIG. 7F. The gene perturbation of PDCD1 in CAR-T cells by PS-Cas9-PDCD-RNP was examined by flow cytometry (left panels), western blotting (middle panel) and DNA sequencing of targeted PDCD1 loci (right panel). Two different PDCD1 gRNAs were initially used to generate PS-Cas9-PDCD1 RNPs (PS-Cas9-PDCD1 RNP1 and PS-Cas9-PDCD1 RNP2). As shown in western blotting, perturbation of the PDCD1 gene mediated by PS-Cas9-PDCD1 RNP2 was better than PS-Cas9-PDCD1 RNP1.

FIGS. 8A-8D. FIG. 8A shows high cell penetrating efficacy (around 90%) and STAT3 gene disruption of PS-Cas9-STAT3-RNP in mouse splenic T cells with (serum+CD3/CD28) or without (serum only) activation, as shown by flow cytometry. One day after T cell activation, PS-Cas9-STAT3 RNP (58.8 pmole) was added to 1×10⁵ mouse splenic T cells. Flow cytometry was performed 4 days after. FIG. 8B shows cell penetration of PS-Cas9-RNP was further confirmed by fluorescent microscopy. Scale bar, 20 μm. FIG. 8C shows similar high cell penetration efficacies of PS-Cas9-STAT3 RNP were observed in resting and activating splenic T cells. FIGS. 8D and 8E show gene perturbation of STAT3 by PS-Cas9-STAT3 RNP in the splenic T cells was examined by qPCR (FIG. 8C) and western blotting (FIG. 8D). qPCR. A forward primer is designed to anneal to the gene perturbation site.

FIGS. 9A and 9B. FIG. 9A shows PS-Cas9-RNP efficiently penetrates primary murine macrophages and disrupts the targeted gene. PS-Cas9-STAT3 RNP (58.8 pmole) was added to 1×10⁵ mouse macrophages. 4 days later, cell penetration of PS-Cas9-STAT3 RNP (PS-Cas9/STAT3 gRNA) in murine macrophages was examined by fluorescent microscopy and the penetration efficacy was shown by a bar graft on the right panel. Shown are mean of the cell penetration in four different fields of the image. Scale bar, 20 μm. FIG. 9B shows STAT3 gene perturbation by PS-Cas9-STAT3 RNP was confirmed by T7E1 assay. *, full length of PCR product amplified from the target gene. Black arrows indicate cleaved DNA products.

FIGS. 10A-10B. PS-Cas9-RNP efficiently penetrates primary human NK cells and disrupts the targeted gene. FIG. 10A shows 1×10⁵ human NK were isolated from donor PBMC cells and incubated with PS-Cas9-STAT3 RNP (58.8 pmole) for 4 days. Cell penetration of PS-Cas9 (no guide RNA) or PS-Cas9-STAT3 RNP (FITC labeled PS-Cas9 with STAT3 gRNA #1) in human NK cells was examined by flow cytometry. FIG. 10B shows STAT3 gene perturbation by PS-Cas9-STAT3 RNP in FITC-positive cells (FITC+) was confirmed by T7E1 assay. *, full length of PCR product amplified from the target gene. The black arrows indicate cleaved DNA products. The gene perturbation rate was quantified by the band intensity of PCR product.

FIGS. 11A-11D. PS-oligo conjugation enables cell penetration of Cas9 RNP and target gene (STAT3) knockout in mouse pancreatic tumor KPC tumor organoid. FIG. 11A shows cell penetration of PS-Cas9-STAT3 RNP (Cy5 labeled) in mouse KPC tumor organoid was visualized after 16 hours incubation by confocal microscopy. Scale bar: 20 μm. FIG. 11B shows targeting Stat3 loci using PS-Cas9-STAT3 RNP. *, PCR products of the target region of Stat3 from KPC organoids incubated with or without PS-Cas9-STAT3 RNP. M, marker. Black arrows indicate the expected cleaved products after T7E1 cleavage assay. FIG. 11C shows relative gene expression of the target Stat3 gene was measured by qPCR after KPC organoids added with or without PS-Cas9-STAT3 RNP for 3 days. Unpaired student t-test; **; P<0.01. FIG. 11D shows fluorescent IHC staining indicates STAT3 expression was significantly decreased in KPC tumor organoid incubated with PS-Cas9-STAT3 RNP for 3 days. Scale bar: 50 μm.

FIG. 12 shows the cell penetration of PS-Cas9-PARG RNP (FITC labeled) in patient-derived ovarian tumor organoid was visualized after 3- or 5-days incubation by confocal microscopy. Scale bar: 100 μm.

FIGS. 13A-13E shows in vivo cell penetration of PS-Cas9-PARG RNP mediated PARG gene perturbation showed significant anti-tumor effects in Olaparib-resistant human ovarian cancer OVCAR8 xenograft tumors. FIG. 13A show tumor bearing NSG mice were treated with 100 μg of PS-Cas9-PARG RNP (Cy5 labeled; 0.58 nmol) or vehicle (HBSS) only once when the tumor size reached to 100 mm³. The images indicate the peritumor injection of PS-Cas9-PARG RNP (Cy5 labeled) in tumor bearing mice after 0.5 hour or day 5 of injection. The mice were euthanized after 5 days of treatment and the size of tumors was shown on the bottom box. FIG. 13B shows the tumor weight was measured and shown in a bar graph. FIG. 13C shows the T7E1 assay was used to measure the PARG gene perturbation in the tumors treated with HBSS or PS-Cas9-PARG RNP. *, full length of PCR product amplified from the target gene. The arrows indicate the cleaved gene products. FIG. 13D shows the effect of gene perturbation by PS-Cas9-PARG RNP was further confirmed by DNA sequencing. The ratio of wildtype and mutant PARG gene was shown in a pie chart. FIG. 13E shows the protein level of PARG in the tumors treated with HBSS or PS-Cas9-PARG RNP were measured by western blotting. The level of actin was detected in parallel as a loading control.

FIG. 14 shows Cas9 with 5 PS oligos (Cas9-5C) vs previously tested Cas9 with one PS oligo (Cas9-C). Our engineered DNA and protein sequences of Cas9-C and Cas9-5C to enable site-specific PS attachments.

FIGS. 15A-15D show Cas9-5C is superior than Cas9-C in both cell penetration and gene disruption. FIG. 15A shows an in vitro Cas9 assay that revealed the enzymatic activity of Cas9 was not interfered by C-terminal Cys-tag and PS-oligo conjugation. Black arrows indicate cleaved DNA products. FIG. 15B. To compare the cell penetrating efficacy of PS-Cas9-C and PS-Cas9-5C (both are FITC labeled), lower dose of PS-conjugated Cas9 RNP (15 pmole) was used for the transfection. flow cytometry showed that cell penetrating efficiency of PS-Cas9-5C-STAT3 RNP was higher than PS-Cas9-C-STAT3 RNP in mouse splenic T cells and lymphocytes. FIGS. 15C and 15D show that STAT3 gene perturbation was examined by western blotting in total cell population (FIG. 15C) and T7E1 assay in sorted FITC-positive cells (FIG. 15D) 4 days post adding PS-Cas9-STAT3 RNP.

FIGS. 16A and 16B. 100 μg of PS-Cas9-5C (0.58 nmole; FITC labeled) was mixed with equal molecules of T cell receptor A/B (TCRA/TCRB) gRNAs to generate PS-Cas9-5C-TCRA/TCRB RNP (FIG. 16A). The cell penetrating efficacy (around 67.1%) of PS-Cas9-5C-TCRA/TCRB RNP (FITC labeled) in human CD4+ T cells was examined by flow cytometry 4 days after adding 0.58 nmole to 1×10⁶ human CD4+ T cells which were isolated from donor PBMCs. The effect of gene perturbation on TCRA/TCRB gene loci was examined by flow cytometry with no detectable PS-Cas9-5C (FITC⁻) and highly PS-Cas9-5C RNP positive (FITC++) CD4 T cells, respectively (FIG. 16B). The expression of TCRα/β was significantly decreased in PS-Cas9-TCRA/TCRB RNP highly positive (FITC++) CD4 T cells. The results also suggest the possibility to generate allogeneic CAR-T cells by using PS-Cas9-TCRA/TCRB RNP.

FIGS. 17A and 17B. PS-Cas9-5C—RNP efficiently penetrates and disrupts murine hippocampal neurons. The neurons were prepared from C57BL/6 mice by using a commercial kit (Pierce Primary Neuron Isolation Kit). 58 pmol of PS-Cas9-5C (10 μg) with (PS-Cas9-5C-STAT3 RNP) or without STAT3 gRNA (PS-Cas9) was added to 1×10⁵ neurons. Three days after adding STAT3 gRNA (PS-Cas9) or PS-Cas9-5C-STAT3 RNP, the efficiency of cell penetration and gene perturbation were examined by flow cytometry (FIG. 17A) and T7E1 assay (FIG. 17B). FIG. 17A. Ctrl: no transfection; PS-Cas9: PS-Cas9 alone; PS-Cas9-STAT3 RNP: PS-Cas9/STAT3 gRNA. FIG. 17B. Left panel: The result of T7E1 assay was shown by DNA electrophoresis. *, full length of PCR product amplified from the target gene. The black arrows indicate cleaved DNA products. Right panel: the level of gene perturbation was quantified by the intensity of DNA bands shown on DNA gel.

FIGS. 18A and 18B. PS-Cas9-5C—RNP efficiently penetrates and disrupts human bone marrow CD34+ hematopoietic stem cells. FIG. 18A. 1×10⁵ human bone marrow CD34+ hematopoietic stem cells (purchased from Zenbio) were incubated with PS-Cas9-STAT3 RNP (58.8 pmole; STAT3 gRNA #3) for 4 days. Cell penetration of PS-Cas9-5C-STAT3 RNP (FITC labeled) in human bone marrow CD34+ hematopoietic stem cells was examined by flow cytometry and the penetration efficacy was shown by a bar graft on the right panel. Shown are mean of three independent experiments. Control: no PS-Cas9. FIG. 18B. Gene perturbation of target gene by PS-Cas9-STAT3 RNP was shown by cell sorting (FITC positive: FITC+; FITC negative: FITC-) and T7E1 assay. *, full length of PCR product amplified from the target gene. Black arrows indicate cleaved DNA products.

FIGS. 19A and 19B. Hybridization of two complementary phosphonothioate oligos enables physical conjugation of an antibody to Cas9, leading to cell-type selective gene perturbation by Cas-9. FIG. 19A. PS-AS-Herceptin is the anti-sense (AS)PS-oligo (Cy5 labeled) conjugated to Herceptin (human anti-Her2 antibody). PS—S-Cas9 is the sense (S)PS-oligo (FITC labeled) conjugated to Cas9. After mixing PS-AS-Herceptin and PS—S-Cas9 at 1:1 ratio, the dimerization of antibody-Cas9 complex was examined by non-denature SDS-PAGE and visualized by fluorescent imaging (Cy5 and FL488). FIG. 19B. Upper panel, the cell penetrating efficacy of PS-Cas9-PS-Herceptin was higher in MCF7-Her2 expressing cells than in MCF7 (Her2-) cells, as shown by flow cytometry. Lower panel, the mean of fluorescent intensity (Cy5) was quantified by a software, FlowJo, and shown in a bar graph.

FIGS. 20A-20C. FIG. 20A. 1×10⁶ Her2 negative MCF7 and Her2 expressing MCF7-Her2 cells were incubated with suboptimal concentration of the indicated PS-Cas9s to discern potential superiority of cell-penetrating Cas9 link to an antibody (30 pmole of PS-Cas9 RNP (STAT3 gRNA #1) or PS-Cas9-PS-Herceptin RNP (STAT3 gRNA #1) were used for the experiments. Upper panels, protein expression of STAT3 was examined by western blotting. Lower panels, the protein band intensity was quantified by a software, ImageJ, and shown in bar graphs. FIG. 20B. The level of Her2 expression in four ovarian cancer cell lines was examined by flow cytometry. SKO3 has the highest level of Her2 expression while OVCAR3 has the lowest level. SKOV3 and OVCAR3 cells were used to transfect PS-Cas9 RNP (STAT3 gRNA #1) or PS-Cas9-PS-Herceptin RNP (STAT3 gRNA #1) for 4 days. Upper right panels, protein expression of STAT3 was examined by western blotting. Lower right panels, the protein band intensity was quantified by Image and shown in bar graphs. FIG. 20C. The efficacy of STAT3 gene perturbation by PS-Cas9 RNP or PS-Cas9-PS-Herceptin in MCF7-Her2+ cells was further confirmed by DNA sequencing. The ratio of wildtype and mutated STAT3 gene was shown in a pie chart.

FIG. 21. Dimerized PS-Cas9 increases Cas9 activities in disrupting targeted gene. Hybridization of two complementary (phosphorothioate) oligos enables two molecules of PS-Cas9 to form a dimer. PS—S-Cas9 is a sense (S)PS-oligo (FITC labeled) conjugated Cas9. PS-AS-Cas9 is an anti-sense (AS)PS-oligo (Cy5 labeled) conjugated Cas9. After mixing PS-AS-Cas9 and PS—S-Cas9 at 1:1 ratio, the dimerization of PS-Cas9 complex was examined by non-denature SDS-PAGE and visualized by fluorescent imaging (Cy5.5 and FL488).

FIGS. 22A and 22B. FIG. 22A shows that PS monomer (PS—S-Cas9-STAT3 RNP1; 100 μg; 0.58 nmole) or dimer (PS—S-AS-Cas9-STAT3 RNP; 50 μg of each PS—S-Cas9-STAT3 RNP1 and PS-AS-Cas9-STAT3 RNP2) of PS-Cas9-STAT3 RNP were used to transfect 1×10⁶ human myeloid cells (CD11b⁺) that was isolated from PBMC of two healthy donors. Two different STAT3 gRNAs (gRNA #1 and #2, with 47 bp between the two) were used to generate PS-Cas9 RNP dimer (paired with sense (S) and anti-sense (AS) PS-oligo). Four days after adding the PS-Cas9 RNP dimer or monomer, cell penetration efficacy was examined by flow cytometry. As shown in the figures, cell penetrating efficacy of monomer PS—S-Cas9-STAT3 RNP (100 μg) in myeloid cells from two different donors was 56.9% and 68.5%, respectively. The penetrating efficacy of PS-Cas9-STAT3 RNP dimer (PS—S-AS-Cas9-STAT3 RNP), under suboptimal experimental conditions (50 μg of each PS—S-Cas9 and PS-AS-Cas9), was 35.5% and 35.1%. FIG. 22B shows a T7E1 assay used to examine the on-target gene perturbation efficacy of monomer (PS—S-Cas9-STAT3 RNP1 or PS—S-Cas9-STAT3 RNP2), two different monomers mix (50 μg of each PS—S-Cas9-STAT3 RNP1 and PS—S-Cas9-STAT3 RNP2; both RNPs are conjugated with sense-PS-oligo) or dimer of PS-Cas9-STAT3 RNP. The results indicate PS—S-AS-Cas9-STAT3 dimer was more efficient than monomer or two-monomer mix of PS-Cas9-STAT3 RNP in STAT3 gene perturbation. *, full length of PCR product amplified from the target gene. Black arrows indicate the cleaved DNA fragments.

FIGS. 23A and 23B. Dimerized PS-dead Cas9 (dCas9) increases gene activation capacity over single PS-dead Cas9. FIG. 23A. Phosphorothioate oligo dimerization enables two molecules of PS-dCas9-VP64 to form a dimer. PS—S-dCas9 is a sense (S)PS-oligo (FITC labeled) conjugated dCas9. PS-AS-dCas9 is an anti-sense (AS)PS-oligo (Cy5 labeled) conjugated dCas9. After mixing PS-AS-dCas9 and PS—S-dCas9 at 1:1 ratio, the dimerization of PS-dCas9 complex was examined by non-denature SDS-PAGE and visualized by fluorescent imaging (Cy5.5 and FL488). FIG. 23B. Functional analysis of monomer and dimer PS-dCas9 in activating gene. Two gRNAs (FIGS. 23A and 23B) designed to target the VEGFA promoter loci are closed to each other. PS—S-dCas9-VP64 was incubated with gRNA A to form PS—S-dCas9-VP64-RNP-A while PS-AS-dCas9-VP64-RNP—B was incubated with gRNA B to form PS-AS-dCas9-VP64 RNP. PS—S-dCas9-VP64 RNP and PS-AS-dCas9-VP64 RNP were mixed at 1:1 ratio to form a dimer complex. 1×10⁶ HEK293 cells were incubated with 100 μg (0.58 nmole) of PS—S-dCas9-VP64-RNP-A, PS-AS-dCas9-VP64-RNP—B or PS—S-AS-dCas9-VP64 RNP-AB dimer for 2 days. The relative VEGFA expression was examined and quantified by qPCR. Shown are mean±SD; n=3; two-tailed student's t test; ***, P<0.005; ****, P<0.001.

FIG. 24 shows that based on off-target gene prediction (IDT online software: www.idtdna.com/pages/products/crispr-genome-editing/alt-r-crispr-cas9-system) of STAT3 gRNA #1 and #2, the off-target gene perturbation by PS-Cas9-STAT3 RNP dimer in comparison to its monomer counter parts (both single monomer and two-monomer mix) was assessed. Off-target gene cutting on Chromosomes 12 and 16 by STAT3 gRNA #1 was detected, while STAT3 gRNA #2 had off-target gene disruption only in Chromosome 1 by the two monomer controls. In contrast, no off-target gene cutting by the PS—S-AS-Cas9-STAT3 dimer was detected. *, full length of PCR product amplified from the target gene. Black arrows indicate the predicted DNA products after T7E1 cleavage.

### DETAILED DESCRIPTION OF THE INVENTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5.6-fused ring heteroarylene refers to two rings fused together, wherein one ring has 5 members and the other ring has 6 members, and wherein at least one ring is a heteroaryl ring. Likewise, a 6.6-fused ring heteroarylene refers to two rings fused together, wherein one ring has 6 members and the other ring has 6 members, and wherein at least one ring is a heteroaryl ring. And a 6.5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

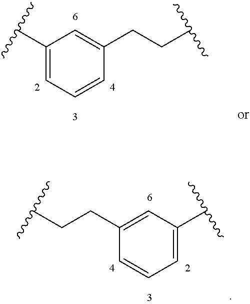 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Where a moiety is substituted with an R substituent, the group may be referred to as “R-substituted.” Where a moiety is R-substituted, the moiety is substituted with at least one R substituent and each R substituent is optionally different. For example, where a moiety herein is R^{1A}-substituted or unsubstituted alkyl, a plurality of R^{1A} substituents may be attached to the alkyl moiety wherein each R^{1A} substituent is optionally different. Where an R-substituted moiety is substituted with a plurality of R substituents, each of the R-substituents may be differentiated herein using a prime symbol (′) such as R′, R″, etc. For example, where a moiety is R^{3A}-substituted or unsubstituted alkyl, and the moiety is substituted with a plurality of R^{3A} substituents, the plurality of R^{3A} substituents may be differentiated as R^{3A′}, R^{3A″}, R^{3A′″}, etc. In some embodiments, the plurality of R substituents is 3.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo,
halogen, —CCl₃, —CBr₃, —CF₃, —CI₃, —CN, —OH, —NH₂, —COOH, —CONH₂, —NO₂, —SH, —S O₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCH Br₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo,
   halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OC HBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo,
      halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —O CHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H,
      —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OC HBr₂,
      —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “chemical linker,” as provided herein, is a covalent linker, anon-covalent linker, a peptide linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof. Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

As used herein, the term “conjugate” refers to the association between atoms or molecules. The association can be direct or indirect. For example, a conjugate between a nucleic acid and a protein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive moieties or functional groups used for conjugate chemistries (including “click chemistries” as known in the art) herein include, for example:
(a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold;
(h) amine or sulfhydryl groups, which can be, for example, acylated, alkylated or oxidized;
(i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds; and
(n) sulfones, for example, vinyl sulfone.

Chemical synthesis of compositions by joining small modular units using conjugate (“click”) chemistry is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’ Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Trisubstituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stockmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry), all of which are hereby incorporated by reference in their entirety and for all purposes.

The reactive functional groups can be chosen such that they do not participate in, or interfere with, the chemical stability of the proteins or nucleic acids described herein. By way of example, the nucleic acids can include a vinyl sulfone or other reactive moiety (e.g., maleimide). Optionally, the nucleic acids can include a reactive moiety having the formula —S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

The term “reactive moiety” as provided herein refers to a chemically functional group of a molecule (e.g., compound or antigen binding domain provided herein), which is capable of forming a covalent or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like) (e.g., covalent or non-covalent bonds) with another reactive moiety of the same or a different molecule. In embodiments, the reactive moiety is a click chemistry reactive group or click chemistry reactive moiety (i.e., a reactive moiety or functional group useable for conjugate chemistries (including “click chemistries” as known in the art)). As described above a click chemistry reactive group is a chemically functional group useful for conjugate chemistry. Thus, in embodiments, the reactive moiety is an azide moiety. In embodiments, the reactive moiety has the structure of —N═N⁺═N⁻. In embodiments, the reactive moiety is alkyne.

In embodiments, the reactive moiety is DBCO. The term “DBCO” as provided herein refers in a customary sense to dibenzocyclooctyl identified by PubChem No. 77078258 or any reactive group including DBCO. In embodiments, the reactive moiety has or includes the structure:

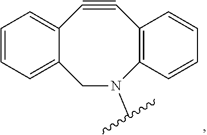 [SMILES: *N1Cc2ccccc2C#Cc2ccccc21]

wherein [symbol] indicates the point of attament to the reminder of the molecule. In embodiments, the reactive moiety is 30 kDa pegylated-DBCO.

In embodiments, the reactive moiety is a trans-cyclooctene (TCO) moiety. The term “TCO” as provided herein refers in a customary sense to trans-cyclooctene identified by PubChem No. 89994470 or any reactive group including TCO. In embodiments, the reactive moiety has or includes the structure:

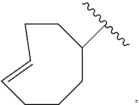 [SMILES: *C1CC/C=C/CCC1]

wherein [symbol] indicates the point of attament to the reminder of the molecule.

In embodiments, the reactive moiety is a tetrazine moiety. The term “tetrazine” as provided herein refers in a customary sense to tetrazine identified by PubChem No. 9263 or any reactive group including tetrazine. In embodiments, the reactive moiety has or includes the structure:

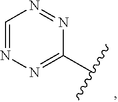 [SMILES: *c1nncnn1]

wherein [symbol] indicates the point of attament to the reminder of the molecule.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded RNA, and hybrid molecules having mixtures of single and double stranded DNA and RNA. Examples of nucleic acids, e.g. polynucleotides, contemplated herein include, but are not limited to, any type of RNA, e.g., mRNA, siRNA, miRNA, sgRNA, and guide RNA and any type of DNA, genomic DNA, plasmid DNA, and minicircle DNA, and any fragments thereof. In embodiments, the nucleic acid is messenger RNA. In embodiments, the messenger RNA is messenger ribonucleoprotein (RNP). The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “phosphorothioate nucleic acid” refers to a nucleic acid in which one or more internucleotide linkages are through a phosphorothioate moiety (thiophosphate) moiety. The phosphorothioate moiety may be a monothiophosphate (—P(O)₃(S)³⁻—) or a dithiophosphate (—P(O)₂(S)₂³⁻—). In embodiments of all the aspects provided herein, the phosphorothioate moiety is a monothiophosphate (—P(O)₃(S)³⁻—). That is, in embodiments of all the aspects provided herein, the phosphorothioate nucleic acid is a monothiophosphate nucleic acid. In embodiments, one or more of the nucleosides of a phosphorothioate nucleic acid are linked through a phosphorothioate moiety (e.g. monothiophosphate) moiety, and the remaining nucleosides are linked through a phosphodiester moiety (—P(O)₄³⁻). In embodiments, one or more of the nucleosides of a phosphorothioate nucleic acid are linked through a phosphorothioate moiety (e.g. monothiophosphate) moiety, and the remaining nucleosides are linked through a methylphosphonate linkage. In embodiments, all the nucleosides of a phosphorothioate nucleic acid are linked through a phosphorothioate moiety (e.g. a monothiophosphate) moiety.

Phosphorothioate oligonucleotides (phosphorothioate nucleic acids) are typically from about 5, 6, 7, 8, 9, 10, 12, 15, 25, 30, 40, 50 or more nucleotides in length, up to about 100 nucleotides in length. Phosphorothioate nucleic acids may also be longer in lengths, e.g., 200, 300, 500, 1000, 2000, 3000, 5000, 7000, 10,000, etc. As described above, in certain embodiments. the phosphorothioate nucleic acids herein contain one or more phosphodiester bonds. In other embodiments, the phosphorothioate nucleic acids include alternate backbones (e.g., mimics or analogs of phosphodiesters as known in the art, such as, boranophosphate, methylphosphonate, phosphoramidate, or O-methylphosphoroamidite linkages (see Eckstein, Oligonucleotides and Analogues: A Practical Approach, Oxford University Press). The phosphorothioate nucleic acids may also include one or more nucleic acid analog monomers known in the art, such as, peptide nucleic acid monomer or polymer, locked nucleic acid monomer or polymer, morpholino monomer or polymer, glycol nucleic acid monomer or polymer, or threose nucleic acid monomer or polymer. Other analog nucleic acids include those with positive backbones; non-ionic backbones, and nonribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. Phosphorothioate nucleic acids and phosphorothioate polymer backbones can be linear or branched. For example, the branched nucleic acids are repetitively branched to form higher ordered structures such as dendrimers and the like.

As used herein, a “phosphorothioate polymer backbone” is a chemical polymer with at least two phosphorothioate linkages (e.g. monothiophosphate) (e.g. linking together sugar subunits, cyclic subunits or alkyl subunits). The phosphorothioate polymer backbone may be a phosphorothioate sugar polymer, which is a phosphorothioate nucleic acid in which one or more (or all) of the chain of pentose sugars lack the bases (nucleobases) normally present in a nucleic acid. The phosphorothioate polymer backbone can include two or more phosphorothioate linkages. In embodiments, the phosphorothioate polymer backbone includes at least 8, 9, 10, 11, 12, 13, 14, 15, 16, 17, 18, 19, 20, 21, 22, 23, 24, 25, 26, 27, 28, 29, or 30 phosphorothioate linkages. In embodiments, the phosphorothioate polymer backbone includes 8, 9, 10, 11, 12, 13, 14, 15, 16, 17, 18, 19, 20, 21, 22, 23, 24, 25, 26, 27, 28, 29, or 30 phosphorothioate linkages. In embodiments, the phosphorothioate nucleic acid includes at least 8, 9, 10, 11, 12, 13, 14, 15, 16, 17, 18, 19, 20, 21, 22, 23, 24, 25, 26, 27, 28, 29, or 30 phosphorothioate linkages. In embodiments, the phosphorothioate nucleic acid includes 8, 9, 10, 11, 12, 13, 14, 15, 16, 17, 18, 19, 20, 21, 22, 23, 24, 25, 26, 27, 28, 29, or 30 phosphorothioate linkages. The phosphorothioate polymer backbone can include 5, 6, 7, 8, 9, 10, 12, 15, 25, 30, 40, 50 or more linkages and can contain up to about 100 phosphorothioate linkages. Phosphorothioate polymer backbones may also contain a larger number of linkages, e.g., 200, 300, 500, 1000, 2000, 3000, 5000, 7000, 10,000, and the like.

The phosphorothioate nucleic acids and phosphorothioate polymer backbones may be partially or completely phosphorothioated. In embodiments, at least 10%, 20%, 30%, 40%, 50%, 60%, 70%, 80%, 90%, 95%, 98% or 100% of the internucleotide linkages of a phosphorothioate nucleic acid are phosphorothioate linkages. In embodiments, 10%, 20%, 30%, 40%, 50%, 60%, 70%, 80%, 90%, 95%, 98% or 100% of the internucleotide linkages of a phosphorothioate nucleic acid are phosphorothioate linkages. For example, 50% or more of the internucleotide linkages of a phosphorothioate nucleic acid can be phosphorothioate linkages. In embodiments, 5%, 10%, 15%, 20%, 25%, 30%, 35%, 40%, 45%, 50%, 55%, 60%, 65%, 70%, 75%, 80%, 85%, 90%, 95%, or 99% of the internucleotide linkages of a phosphorothioate nucleic acid are phosphorothioate linkages. In embodiments, 50%, 55%, 60%, 65%, 70%, 75%, 80%, 85%, 90%, 95%, or 99% of the internucleotide linkages of a phosphorothioate nucleic acid are phosphorothioate linkages. In embodiments, 75%, 80%, 85%, 90%, 95%, or 99% of the internucleotide linkages of a phosphorothioate nucleic acid are phosphorothioate linkages. In embodiments, 90%, 95%, or 99% of the internucleotide linkages of a phosphorothioate nucleic acid are phosphorothioate linkages. In embodiments, the remaining internucleotide linkages are phosphodiester linkages. In embodiments, the remaining internucleotide linkages are methylphosphonate linkages. In embodiments, 100% of the internucleotide linkages of the phosphorothioate nucleic acids are phosphorothioate linkages. Similarly, 5%, 10%, 15%, 20%, 25%, 30%, 35%, 40%, 45%, 50%, 55%, 60%, 65%, 70%, 75%, 80%, 85%, 90%, 95%, or 99%, of the intersugar linkages in a phosphorothioate polymer backbone can be phosphorothioate linkages. In embodiments, 50%, 55%, 60%, 65%, 70%, 75%, 80%, 85%, 90%, 95%, or 99%, of the intersugar linkages in a phosphorothioate polymer backbone can be phosphorothioate linkages. In embodiments, 75%, 80%, 85%, 90%, 95%, or 99%, of the intersugar linkages in a phosphorothioate polymer backbone can be phosphorothioate linkages. In embodiments, 90%, 95%, or 99%, of the intersugar linkages in a phosphorothioate polymer backbone can be phosphorothioate linkages. In embodiments, the remaining internucleotide linkages are phosphodiester linkages. In embodiments, the remaining internucleotide linkages are methylphosphonate linkages. In embodiments, 100% of the intersugar linkages of the phosphorothioate polymer backbone are phosphorothioate linkages.

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art. In embodiments, the phosphorothioate nucleic acid or phosphorothioate polymer backbone is connected to a detectable label through a chemical linker.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

The phosphorothioate nucleic acids and phosphorothioate polymer backbones provided herein can include one or more reactive moieties, e.g., a covalent reactive moiety. A reactive moiety may be attached to the remainder of the phosphorothioate nucleic acids and phosphorothioate polymer backbones using any appropriate linker, such as a polymer linker known in the art or alternatively a polyethylene glycol linker or equivalent. The linker may, in embodiments, include (i.e. be attached to) a detectable label as described herein. As used herein, the term “covalent reactive moiety” refers to a chemical moiety capable of chemically reactive with an amino acid of a gene editing agent or targeting agent, as described herein, to form a covalent bond and, thus, a conjugate as provided herein.

Nucleic acids can include nonspecific sequences. As used herein, the term “nonspecific sequence” refers to a nucleic acid sequence which does not encode for a particular function. For example, a nonspecific sequence may contain a series of residues that are not designed to be complementary to or are only partially complementary to any other nucleic acid sequence. A nonspecific sequence may be a sequence that does not encode for a functional nucleic acid or protein. In embodiments, a nonspecific sequence is a sequence of a nucleic acid that includes nucleotides randomly attached to each other. In embodiments, a nonspecific sequence does not encode for a biological function. A nonspecific sequence may be referred to as a “scrambled” sequence (e.g., scrambled nucleic acid sequence). A scrambled sequence (e.g., scrambled nucleic acid sequence) may be created by a software tool to create the sequence scramble as negative control for a functional sequence (e.g., nucleic acid sequence). A nonspecific sequence may be generated by randomly attaching nucleotides to each other, without a certain order. By way of example, a nonspecific sequence (e.g., nucleic acid sequence) is a sequence (e.g., nucleic acid sequence) that does not function as an inhibitory nucleic acid when contacted with a cell or organism. The phosphorothioate nucleic acids provided herein are nonspecific nucleic acid sequences, which do not include specific sequence information and/or do not encode for a functional nucleic acid or protein. Therefore, the function of the phosphorothioate nucleic acids provided herein is independent from their sequence, but dependent on whether they include phosphorothioate linkages.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions). Nucleic acids that are substantially complementary or complementary as provided herein if they include sequences that are at least 50%, 60%, 65%, 70%, 75%, 80%, 85%, 90%, 95%, 97%, 98%, 99%, or 100% complementary over a region of 5, 6, 7, 8, 9, 10, 11, 12, 13, 14, 15, 16, 17, 18, 19, 20, 21, 22, 23, 24, 25, 30, 35, 40, 45, 50, or more contiguous nucleotides.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “genome editing” or “gene editing” as provided herein refer to stepwise processes involving enzymes such as polymerases, ligases, exonucleases, endonucleases or the like or a combinations thereof. For example, gene editing may include processes where a nucleic acid molecule is cleaved, nucleotides at the cleavage site or in close vicinity thereto are excised, new nucleotides are newly synthesized and the cleaved strands are ligated. A “gene editing agent” provided herein therefor includes any protein or enzyme or combination thereof capable of editing a genomic sequence. Gene editing may result in the insertion of nucleotides, deletion of nucleotides or mutations (point mutation) at a target sequence. As a result of gene editing one or more nucleotides may be inserted, deleted or replaced by one or more chemically different nucleotides at a target sequence (target locus). Thus, cells modified using various gene editing methods (e.g, methods using a homologous recombination (HR), nonhomologous end joining (NHEJ), transposon-mediated system, loxP-Cre system, CRISPR/Cas9 or TALEN) are within the scope of the disclosure. In embodiments, one or more target locus within the subject's genomic DNA is targeted and modified. A treatment method comprises gene editing tools available in the art, e.g., CRISPR, zinc finger nucleases, meganucleases, where a target DNA locus, e.g., a gene of interest, is modified to create a mutation in the gene product, e.g., a protein or enzyme, with reduced activity or no activity (loss-of-function mutation).

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

PAM refers to “protospacer adjacent motif”. These sites are generally 2-6 base pair DNA sequences that are adjacent to DNA sequence bound by Cas9. Thus, in some instances, DNA-binding modulation-enhancing agents other than Cas9 might be used and in other instances a single Cas9/RNA complex might be used as a DNA-binding modulation-enhancing agent (either alone or in conjunction with a different DNA-binding modulation-enhancing agent).

A “guide RNA” or “gRNA” as provided herein refers to a ribonucleotide sequence capable of binding a nucleoprotein, thereby forming ribonucleoprotein complex. In embodiments, the guide RNA includes one or more RNA molecules. In embodiments, the gRNA includes a nucleotide sequence complementary to a target site (e.g., target sequence). The complementary nucleotide sequence included in the gRNA may mediate binding of the ribonucleoprotein complex to said target sequence thereby providing the sequence specificity of the ribonucleoprotein complex. Thus, in embodiments, the guide RNA is complementary to a target nucleic acid (e.g., a target sequence). In embodiments, the guide RNA binds a target nucleic acid sequence (e.g., a target sequence). In embodiments, the guide RNA is complementary to a CRISPR nucleic acid sequence. In embodiments, the complement of the guide RNA has a sequence identity of about 50%, 55%, 60%, 65%, 70%, 75%, 80%, 85%, 90%, 95%, 96%, 97%, 98% or 99% to a target nucleic acid (e.g., a target sequence). A target nucleic acid sequence as provided herein is a nucleic acid sequence expressed by a cell. In embodiments, a target nucleic acid sequence as provided herein is a target sequence. In embodiments, the target sequence is an exogenous nucleic acid sequence. In embodiments, the target sequence is an endogenous nucleic acid sequence. In embodiments, the target sequence forms part of a cellular gene. Thus, in embodiments, the guide RNA is complementary to a cellular gene or fragment thereof. In embodiments, the guide RNA is about 50%, 55%, 60%, 65%, 70%, 75%, 80%, 85%, 90%, 95%, 96%, 97%, 98% or 99% to the target sequence. In embodiments, the guide RNA is about 50%, 55%, 60%, 65%, 70%, 75%, 80%, 85%, 90%, 95%, 96%, 97%, 98% or 99% complementary to the sequence of a cellular gene. In embodiments, the guide RNA binds a cellular gene sequence.

In embodiments, the guide RNA is a single-stranded ribonucleic acid. In embodiments, the guide RNA is about 10, 20, 30, 40, 50, 60, 70, 80, 90, 100 or more nucleic acid residues in length. In embodiments, the guide RNA is from about 10 to about 30 nucleic acid residues in length. In embodiments, the guide RNA is about 20 nucleic acid residues in length. In embodiments, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, the guide RNA is from 10 to 15, 10 to 20, 10 to 30, 10 to 40, or 10 to 50 residues in length.

In general, a guide RNA is any polynucleotide sequence having sufficient complementarity with a target polynucleotide sequence to hybridize with the target sequence (e.g., a genomic or mitochondrial DNA target sequence) and direct sequence-specific binding of a RNA-guided DNA endonuclease to the target sequence. In embodiments, the degree of complementarity between a guide RNA and its corresponding target sequence, when optimally aligned using a suitable alignment algorithm, is about or more than about 50%, 60%, 75%, 80%, 85%, 90%, 95%, 97.5%, 99%, or more. In embodiments, the degree of complementarity between a guide RNA and its corresponding target sequence, when optimally aligned using a suitable alignment algorithm, is at least about 80%, 85%, 90%, 95%, or 100%. In embodiments, the degree of complementarity is at least 90%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In embodiments, a guide RNA is about or more than about 10, 20, 30, 35, 40, 45, 50, 75, or more nucleotides in length. In embodiments, a guide RNA is about 10 to about 50, about 15 to about 30, or about 20 to about 25 nucleotides in length. In embodiments, a guide RNA is less than about 75, 50, 45, 40, 35, 30, 25, 20, 15, 12, or fewer nucleotides in length. In embodiments, the guide RNA is about or more than about 20 nucleotides in length. The ability of a guide RNA to direct sequence-specific binding of a complex (e.g., CRISPR complex) to a target sequence may be assessed by any suitable assay. For example, the components of a CRISPR system sufficient to form a complex (e.g., CRISPR complex), including the guide RNA to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a complex (e.g., CRISPR complex), including the guide RNA to be tested and a control guide RNA different from the test guide RNA, and comparing binding or rate of cleavage at the target sequence between the test and control guide RNA reactions. Other assays are possible, and will occur to those skilled in the art.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, In embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 15 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Nat. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

A “therapeutic antibody” as provided herein refers to any antibody or functional fragment thereof (e.g., a nanobody) that is used to treat cancer, autoimmune diseases, transplant rejection, cardiovascular disease or other diseases or conditions such as those described herein. Non-limiting examples of therapeutic antibodies include murine antibodies, murinized or humanized chimera antibodies or human antibodies including, but not limited to, Erbitux (cetuximab), ReoPro (abciximab), Simulect (basiliximab), Remicade (infliximab); Orthoclone OKT3 (muromonab-CD3); Rituxan (rituximab), Bexxar (tositumomab) Humira (adalimumab), Campath (alemtuzumab), Simulect (basiliximab), Avastin (bevacizumab), Cimzia (certolizumab pegol), Zenapax (daclizumab), Soliris (eculizumab), Raptiva (efalizumab), Mylotarg (gemtuzumab), Zevalin (ibritumomab tiuxetan), Tysabri (natalizumab), Xolair (omalizumab), Synagis (palivizumab), Vectibix (panitumumab), Lucentis (ranibizumab), and Herceptin (trastuzumab).

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

For specific proteins described herein (e.g., Cas9, Cpf1, TALEN, etc.), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In embodiments, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference or functional fragment or homolog thereof.

The term “RNA-guided DNA endonuclease” and the like refer, in the usual and customary sense, to an enzyme that cleaves a phosphodiester bond within a DNA polynucleotide chain, wherein the recognition of the phosphodiester bond is facilitated by a separate RNA sequence (for example, a single guide RNA or a pegRNA).

A “pegRNA” or “prime editing guide RNA” is a ribonucleic acid molecule capable of (i) hybridizing to a target nucleotide sequence that is intended to be edited, and (ii) which encodes new genetic information that replaces the target sequence or portions thereof (at least one nucleotide (e.g., 2, 3, 4, 5, 6)). The pegRNA includes an extended single guide RNA (sgRNA) including a primer binding site (PBS) and a reverse transcriptase (RT) template sequence. During DNA editing, the primer binding site allows the 3′ end of the nicked DNA strand to hybridize to the pegRNA, while the RT template serves as a template for the synthesis of edited genetic information.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system Both type II and type V systems are included in Class II of the CRISPR-Cas system.

The term “nuclease-deficient RNA-guided DNA endonuclease enzyme” or “nuclease-deficient RNA-guided DNA endonuclease” refer, in the usual and customary sense, to an RNA-guided DNA endonuclease (e.g., a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., sgRNA), but lacks significant endonuclease activity (e.g. any amount of detectable endonuclease activity). In embodiments, the nuclease-deficient RNA-guided DNA endonuclease is dCas9, dCpf1, ddCpf1, a nuclease-deficient Cas9 variant, a nuclease-deficient Class II CRISPR endonuclease, a zinc finger domain, a leucine zipper domain, a winged helix domain, TAL effector, a helix-turn-helix motif, a helix-loop-helix domain, an HMB-box domain, a Wor3 domain, an OB-fold domain, an immunoglobulin domain, or a B3 domain. In embodiments, the nuclease-deficient RNA-guided DNA endonuclease enzyme is a zinc finger domain, a leucine zipper domain, a winged helix domain, TAL effector, a helix-turn-helix motif, a helix-loop-helix domain, an HMB-box domain, a Wor3 domain, an OB-fold domain, an immunoglobulin domain, or a B3 domain. In embodiments, the nuclease-deficient RNA-guided DNA endonuclease is a zinc finger domain. In embodiments, the nuclease-deficient RNA-guided DNA endonuclease is a leucine zipper domain. In embodiments, the nuclease-deficient RNA-guided DNA endonuclease is a winged helix domain. In embodiments, the nuclease-deficient RNA-guided DNA endonuclease is a TAL effector. In embodiments, the nuclease-deficient RNA-guided DNA endonuclease is a helix-loop-helix domain. In embodiments, the nuclease-deficient RNA-guided DNA endonuclease is an HMB-box domain. In embodiments, the nuclease-deficient RNA-guided DNA endonuclease is a Wor3 domain. In embodiments, the nuclease-deficient RNA-guided DNA endonuclease is an OB-fold domain. In embodiments, the nuclease-deficient RNA-guided DNA endonuclease is an immunoglobulin domain. In embodiments, the nuclease-deficient RNA-guided DNA endonuclease is a B3 domain. In embodiments, the nuclease-deficient RNA-guided DNA endonuclease is dCas9, ddCpf1, a nuclease-deficient Cas9 variant, or a nuclease-deficient Class II CRISPR endonuclease.

In embodiments, the nuclease-deficient RNA-guided DNA endonuclease is dCas9. The terms “dCas9” or “dCas9 protein” as referred to herein is a Cas9 protein in which both catalytic sites for endonuclease activity are defective or lack activity. In embodiments, the dCas9 protein has mutations at positions corresponding to D10A and H840A of S. pyogenes Cas9. In embodiments, the dCas9 protein lacks endonuclease activity due to point mutations at both endonuclease catalytic sites (RuvC and HNH) of wild type Cas9. The point mutations can be D10A and H840A. In embodiments, the dCas9 has substantially no detectable endonuclease (e.g., endodeoxyribonuclease) activity. In embodiments, the dCas9 has substantially no detectable endonuclease (e.g., endodeoxyribonuclease) activity.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. In embodiments, the Cas9 protein has at least 75% sequence identity to the amino acid sequence of the protein identified by the UniProt reference number Q99ZW2. In embodiments, the Cas9 protein has at least 80% sequence identity to the amino acid sequence of the protein identified by the UniProt reference number Q99ZW2. In embodiments, the Cas9 protein has at least 85% sequence identity to the amino acid sequence of the protein identified by the UniProt reference number Q99ZW2. In embodiments, the Cas9 protein has at least 90% sequence identity to the amino acid sequence of the protein identified by the UniProt reference number Q99ZW2. In embodiments, the Cas9 protein has at least 95% sequence identity to the amino acid sequence of the protein identified by the UniProt reference number Q99ZW2.

In embodiments, the nuclease-deficient RNA-guided DNA endonuclease is “ddCpf1” or “ddCas12a”. The terms “DNAse-dead Cpf1” or “ddCpf1” refer to mutated Acidaminococcus sp. Cpf1 (AsCpf1) resulting in the inactivation of Cpf1 DNAse activity. In embodiments, ddCpf1 includes an E993A mutation in the RuvC domain of AsCpf1. In embodiments, the ddCpf1 has substantially no detectable endonuclease (e.g., endodeoxyribonuclease) activity.

In embodiments, the nuclease-deficient RNA-guided DNA endonuclease is dLbCpf1. The term “dLbCpf1: refers to mutated Cpf1 from Lachnospiraceae bacterium ND2006 (LbCpf1) that lacks DNAse activity. In embodiments, dLbCpf1 includes a D832A mutation. In embodiments, the dLbCpf1 has substantially no detectable endonuclease (e.g., endodeoxyribo-nuclease) activity.

In embodiments, the nuclease-deficient RNA-guided DNA endonuclease is dFnCpf1. The term “dFnCpf1” refers to mutated Cpf1 from Francisella novicida U112 (FnCpf1) that lacks DNAse activity. In embodiments, dFnCpf1 includes a D917A mutation. In embodiments, the dFnCpf1 has substantially no detectable endonuclease (e.g., endodeoxyribo-nuclease) activity.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In embodiments, the Cpf1 protein is substantially identical to the protein identified by the UniProt reference number U2UMQ6 or a variant or homolog having substantial identity thereto. In embodiments, the Cpf1 protein is identical to the protein identified by the UniProt reference number U2UMQ6. In embodiments, the Cpf1 protein has at least 75% sequence identity to the amino acid sequence of the protein identified by the UniProt reference number U2UMQ6. In embodiments, the Cpf1 protein has at least 80% sequence identity to the amino acid sequence of the protein identified by the UniProt reference number U2UMQ6. In embodiments, the Cpf1 protein is identical to the protein identified by the UniProt reference number U2UMQ6. In embodiments, the Cpf1 protein has at least 85% sequence identity to the amino acid sequence of the protein identified by the UniProt reference number U2UMQ6. In embodiments, the Cpf1 protein is identical to the protein identified by the UniProt reference number U2UMQ6. In embodiments, the Cpf1 protein has at least 90% sequence identity to the amino acid sequence of the protein identified by the UniProt reference number U2UMQ6. In embodiments, the Cpf1 protein is identical to the protein identified by the UniProt reference number U2UMQ6. In embodiments, the Cpf1 protein has at least 95% sequence identity to the amino acid sequence of the protein identified by the UniProt reference number U2UMQ6.

An “Argonaut” or “Argonaut protein” as referred to herein includes any of the recombinant or naturally-occurring forms of Argonaut (Natronobacterium gregoryi) endonuclease or variants or homologs thereof that maintain Argonaut endonuclease enzyme activity (e.g. within at least 50%, 80%, 90%, 95%, 96%, 97%, 98%, 99% or 100% activity compared to Argonaut). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Argonaut protein. In embodiments, the Argonaut protein is substantially identical to the protein identified by the UniProt reference number A0A172MAH6 or a variant or homolog having substantial identity thereto.

In embodiments, the nuclease-deficient RNA-guided DNA endonuclease is a nuclease-deficient Class II CRISPR endonuclease. The term “nuclease-deficient Class II CRISPR endonuclease” as used herein refers to any Class II CRISPR endonuclease having mutations resulting in reduced, impaired, or inactive endonuclease activity.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC) can be distinguished. Embryonic stem cells reside in the blastocyst and give rise to embryonic tissues, whereas somatic stem cells reside in adult tissues for the purpose of tissue regeneration and repair. A “neural stem cell” as provided herein refers to a stem cell capable to self-renew through mitotic cell division and to differentiate into a neural cell (e.g., glia cell, neuron, astrocyte, oligodendrocyte).

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetofection and electroporation. For viral-based methods, any useful viral vector can be used in the methods described herein. Examples of viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a fusion protein as provided herein and a nucleic acid sequence (e.g., target DNA sequence).

As defined herein, the term “inhibition”, “inhibit”, “inhibiting,” “repression,” repressing,” “silencing,” “silence” and the like when used in reference to a composition as provided herein (e.g., fusion protein, complex, nucleic acid, vector) refer to negatively affecting (e.g., decreasing) the activity (e.g., transcription) of a nucleic acid sequence (e.g., decreasing transcription of a gene) relative to the activity of the nuclei acid sequence (e.g., transcription of a gene) in the absence of the composition (e.g., fusion protein, complex, nucleic acid, vector). In embodiments, inhibition refers to reduction of a disease or symptoms of disease (e.g., cancer). Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In embodiments, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “target polynucleotide sequence” or “target sequence” as provided herein is a nucleic acid sequence present in, or expressed by, a cell, to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. In embodiments, the target polynucleotide sequence is an exogenous nucleic acid sequence. In embodiments, the target polynucleotide sequence is an endogenous nucleic acid sequence.

The target polynucleotide sequence or target sequence may be any region of the polynucleotide (e.g., DNA sequence) suitable for epigenome editing. In embodiments, the target polynucleotide sequence is part of a gene. In embodiments, the target polynucleotide sequence is part of a transcriptional regulatory sequence. In embodiments, the target polynucleotide sequence is part of a promoter, enhancer or silencer. In embodiments, the target polynucleotide sequence is part of a promoter. In embodiments, the target polynucleotide sequence is part of an enhancer. In embodiments, the target polynucleotide sequence is part of a silencer.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g. leukemia, lymphoma, B cell lymphoma, or multiple myeloma)) means that the disease (e.g. cancer, (e.g. leukemia, lymphoma, B cell lymphoma, or multiple myeloma)) is caused by (in whole or in part), or a symptom of the disease is caused by (in whole or in part) the substance or substance activity or function.

As used herein, the terms “cell-penetrating” or “cell-penetration” refer to the ability of a molecule (e.g., a protein) to pass from the extracellular environment into a cell in a significant or effective amount. Thus, a cell-penetrating conjugate is a molecule that passes from the extracellular environment, through the membrane, and into a cell.

As used herein, the terms “non-cell penetrating” or “non-cell penetration” refers to the inability of a molecule (e.g., a protein or peptide) to pass from the extracellular environment into a cell in a significant or effective amount. Thus, non-cell penetrating peptides or proteins generally are not capable of passing from the extracellular environment, through the cell membrane, and into a cell in order to achieve a significant biological effect on a population of cells, organ or organism. The term does not exclude the possibility that one or more of the small number of peptides or proteins may enter the cell. However, the term refers to molecules that are generally not able to enter a cell from the extracellular environment to a significant degree. Examples of non-cell penetrating molecules and substances include, but are not limited to, large molecules such as, for example, high molecular weight proteins. Peptides or proteins can be determined to be non-cell penetrating using methods known to those of skill in the art. By way of example, a peptide or protein can be fluorescently labeled and the ability of the peptide or protein to pass from the extracellular environment into the cell can be determined in vitro by flow cytometric analysis or confocal microscopy.

As used herein, “molecular weight” (M.W.) or “molecular mass” refers to the sum of the atomic weights of all the atoms in a molecule. With respect to molecules, a molecule with a high molecular weight typically has a molecular weight of 25 kDa or more. By way of example, a high molecular weight protein can have a M.W. from about 25 kDa to 1000 kDa or more.

As used herein, the term “intracellular” means inside a cell. As used herein, an “intracellular target” is a target, e.g., nucleic acid, polypeptide or other molecule (e.g., carbohydrate) that is located inside of a cell and is a target to which the non-cell penetrating proteins provided herein bind. Binding can be direct or indirect. In embodiments, the non-cell penetrating protein selectively binds the intracellular target. The terms “selectively binds,” “selectively binding,” or “specifically binding” refer to an agent (e.g., a non-cell penetrating protein) binding one agent (e.g., intracellular target) to the partial or complete exclusion of other agents. By binding is meant a detectable binding at least about 1.5 times the background of the assay method. For selective or specific binding such a detectable binding can be detected for a given agent but not a control agent. Alternatively, or additionally, the detection of binding can be determined by assaying the presence of down-stream molecules or events.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia lymphoblastic leukemia, chronic lymphocytic leukemia, hairy cell leukemia cancer cell), lymphoma (e.g., mantle cell lymphoma (MCL), follicular lymphoma, diffuse large B-cell lymphoma, marginal zone lymphoma, Burkitt's lymphoma), head and neck cancer, colorectal cancer, prostate cancer, pancreatic cancer, melanoma, breast cancer, neuroblastoma). For example certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### Complexes

Provided herein are, inter alia, complexes useful for editing (e.g., repairing, modifying) DNA in a cell in vitro and in vivo. The complexes provided herein include, inter alia, a DNA editing agent bound to a phosphorothioate nucleic acid through a chemical linker. The chemical linker (e.g., disulfide linker) may be a linker that dissociates once the complex has entered the inside of the cell, thereby releasing the DNA editing agent and allowing the DNA editing agent to access and edit a cellular target sequence. The complexes provided herein exhibit high cellular internalization efficiency and editing efficacy and therefore provide for useful therapeutic and diagnostic tools. The complexes provided herein are furthermore highly target specific and lack unspecific off-target activity. For targeted delivery into cells, the complexes provided herein may be attached to targeting agents (e.g., antibodies, nucleic acids) that specifically bind a surface marker on the cell to which the complex should be delivered. Through hybridization of two complementary phosphorothioated nucleic acid strands, the first of which is attached through a first chemical linker to the editing agent and the second of which is attached through a second chemical linker to the targeting agent, the editing agent and the targeting agent are bound together. Furthermore, to enhance site specific editing activity, complexes are provided that include two editing agents bound to each other through a phosphorothioate nucleic acid. Through hybridization of two complementary phosphorothioated nucleic acid strands, the first of which is attached through a first chemical linker to a first editing agent and the second of which is attached through a second chemical linker to a second editing agent, the first editing agent and the second editing agent are bound together.

In an aspect, a complex for delivering a gene editing agent to a cell is provided. The complex includes a gene editing agent covalently bound to a phosphorothioate nucleic acid through a chemical linker. In embodiments, the gene editing agent includes a cysteine and the phosphorothioate nucleic acid includes a thiol moiety covalently bound to the gene editing agent through a disulfide linkage between the cysteine and the thiol moiety. In embodiments, the phosphorothioate nucleic acid is bound to the C-terminus of the gene editing agent.

In embodiments, the chemical linker is a covalent linker.

In embodiments, the chemical linker is L¹. In embodiments, L¹ may be a bond, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

In embodiments, L¹ may be a bond, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₈) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered) heteroarylene.

In embodiments, L¹ is substituted or unsubstituted C₁-C₁₀ alkylene. In embodiments, L¹ is substituted or unsubstituted C₂-C₁₀ alkylene. In embodiments, L¹ is substituted or unsubstituted C₄-C₁₀ alkylene. In embodiments, L¹ is substituted or unsubstituted C₆-C₁₀ alkylene. In embodiments, L¹ is substituted or unsubstituted C₅-C₁₀ alkylene. In embodiments, L¹ is substituted or unsubstituted C₂-C₈ alkylene. In embodiments, L¹ is substituted or unsubstituted C₄-C₈ alkylene. In embodiments, L¹ is substituted C₁-C₁₀ alkylene. In embodiments, L¹ is substituted C₂-C₁₀ alkylene. In embodiments, L¹ is substituted C₄-C₁₀ alkylene. In embodiments, L¹ is substituted C₆-C₁₀ alkylene. In embodiments, L¹ is substituted C₈-C₁₀ alkylene. In embodiments, L¹ is substituted C₂-C₈ alkylene. In embodiments, L¹ is substituted C₄-C₈ alkylene. In embodiments, L¹ is substituted C₆ alkylene.

In embodiments, L¹ is substituted or unsubstituted 1 to 10 membered heteroalkylene. In embodiments, L¹ is substituted or unsubstituted 2 to 10 membered heteroalkylene. In embodiments, L¹ is substituted or unsubstituted 4 to 10 membered heteroalkylene. In embodiments, L¹ is substituted or unsubstituted 6 to 10 membered heteroalkylene. In embodiments, L¹ is substituted or unsubstituted 8 to 10 membered heteroalkylene. In embodiments, L¹ is unsubstituted 1 to 10 membered heteroalkylene. In embodiments, L¹ is unsubstituted 4 to 10 membered heteroalkylene. In embodiments, L¹ is unsubstituted 2 to 8 membered heteroalkylene. In embodiments, L¹ is unsubstituted 2 to 6 membered heteroalkylene. In embodiments, L¹ is unsubstituted 7 membered heteroalkylene.

In embodiments, the chemical linker is a pH-sensitive linker. A “pH-sensitive linker” is a chemical linker, whose structural integrity is pH dependent. Therefore, a pH-sensitive linker may bind two components (e.g., a phosphorothioate nucleic acid and a gene editing agent) at a first pH, but may not bind these same components at a second pH. The chemical structure of the pH-sensitive linker at the second pH is different from its structure at the first pH and does not allow for the pH-sensitive linker to bind the two components together.

The gene editing complexes provided herein include a gene editing agent (e.g., a first or a second gene editing agent), or a gene editing agent and a targeting agent covalently bound through a disulfide linkage to a phosphorothioate nucleic acid. The disulfide linkage may be formed between an amino acid residue of the gene editing agent or targeting agent and a phosphorothioate nucleic acid that includes a thiol moiety. The amino acid residue may be a cysteine, a protected cysteine (a cysteine covalently attached to a protecting group) or an arginine substituted with a thiol-substituent (an octyl-thiol-substituted arginine). In embodiments, the chemical linker is a thioester linker. In embodiments, the chemical linker is a disulfide linker.

The gene editing agent or targeting agent provided herein are covalently attached to one or more phosphorothioate nucleic acid through a disulfide linkage. A “disulfide linkage”, “disulfide bridge” or “disulfide bond” as provided herein refers to a covalent bond formed by reacting two thiol moieties. The first of the two reacting thiol moieties forms part of the gene editing agent or the targeting agent provided herein and the second thiol moiety forms part of the one or more phosphorothioate nucleic acids provided herein. In embodiments, the gene editing complex provided herein has the structure of R^{A}—S—S—R^{B}, wherein R^{A} is a gene editing agent (e.g., first gene editing agent or second gene editing agent) and R^{B} is a phosphorothioate nucleic acid. The disulfide linkage is formed between a cysteine of the gene editing agent (e.g., first gene editing agent or second gene editing agent) (first or second cysteine) and a thiol reactive moiety of the phosphorothioate nucleic acid.

A “thiol reactive moiety” as provided herein is a chemical moiety which includes a sulfur atom, wherein the sulfur may form part of a disulfide linkage, and may also be referred to herein as a “sulfur-containing reactive moiety.” A disulfide linkage as referred to herein includes a sulfur atom derived from a reacted —SH substituent (i.e., a thiol group or thiol substituent which is a group or substituent including a thiol). Thus, the thiol reactive moiety provided herein may also be referred to as sulfur reactive moiety. In embodiments, the sulfur atom forms part of an amino acid (e.g., a cysteine side chain). In embodiments, the sulfur atom forms part of a substituted amino acid side chain (e.g., a substituted arginine side chain). Where the sulfur atom forms part of a substituted amino acid side chain, the amino acid side chain may be substituted with a substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, the substituted amino acid side chain is substituted with octyl-thiol. In embodiments, the octyl-thiol has the formula:

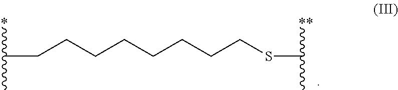

In formula (III) * denotes the attachment point with the amino acid side chain and ** denotes the point of attachment with the first cysteine. In embodiments, the substituted amino acid side chain is a substituted arginine side chain. In embodiments, the substituted arginine includes the compound of formula (III). In embodiments, the substituted arginine is an octyl-thiol-substituted arginine. In embodiments, the octyl-thiol-substituted arginine includes the compound of formula (III). In embodiments, the thiol side chain amino acid is a cysteine.

As discussed above, polymer backbones contain the same structure (i.e., contains a chain of two or more sugar residues linked together) as a nucleic acid sequence with the exception that the polymer backbone lacks the bases normally present in a nucleic acid sequence.

The phosphorothioate nucleic acids or phosphorothioate polymer backbones can be of any appropriate length. In embodiments, the phosphorothioate nucleic acid or phosphorothioate polymer backbone is independently 10, 20, 30, 40, 50, 60, 70, 80, 90, 100 or more nucleic acid residues or sugar residues in length. In embodiments, the phosphorothioate nucleic acid or phosphorothioate polymer backbone is independently from 10 to 30 residues in length. Thus, the length of each nucleic acid or polymer backbone can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, the phosphorothioate nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acid residues in length.

In embodiments, the phosphorothioate nucleic acid or phosphorothioate polymer backbone is independently from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, the phosphorothioate nucleic acid is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 residues in length. In embodiments, the phosphorothioate nucleic acid or phosphorothioate polymer backbone is independently from 10 to 15, 10 to 20, 10 to 30, 10 to 40, or 10 to 50 residues in length. In embodiments, the phosphorothioate nucleic acid is from 10 to 15, 10 to 20, 10 to 30, 10 to 40, or 10 to 50 residues in length.

In embodiments, the length of one phosphorothioate nucleic acid or phosphorothioate polymer backbone differs from another phosphorothioate nucleic acid or phosphorothioate polymer backbone. By way of example, if two phosphorothioate nucleic acids or phosphorothioate polymer backbones are attached to a gene editing agent (e.g., first or second gene editing agent) or a targeting agent as provided herein, the first phosphorothioate nucleic acid or phosphorothioate polymer backbone can be of one length (e.g., 22 residues) and the second phosphorothioate nucleic acid or phosphorothioate polymer backbone can be of a different length (e.g. 25 residues). Thus, if a plurality of phosphorothioate nucleic acids and phosphorothioate polymer backbones are attached to a gene editing agent (e.g., first or second gene editing agent) or a targeting agent as provided herein, the phosphorothioate nucleic acids and phosphorothioate polymer backbones can have a number of different lengths, e.g., ranging from 10 to 30 residues in length.

In embodiments a plurality of phosphorothioate nucleic acids or phosphorothioate polymer backbones are attached to the gene editing agent (e.g., first or second gene editing agent) or the targeting agent. In embodiments, 1, 2, 3, 4, 5, 6, 7, 8, 9, 10, 15, 20, 25, or more phosphorothioate nucleic acids or phosphorothioate polymer backbones are attached to the gene editing agent (e.g., first or second gene editing agent) or the targeting agent. In embodiments, the attachment is covalent. The attachment may be non-covalent. The phosphorothioate nucleic acids or phosphorothioate polymer backbones can be independently attached to a lysine, arginine, cysteine, or histidine of the gene editing agent (e.g., first or second gene editing agent) or the targeting agent. In embodiments, each phosphorothioate nucleic acid or phosphorothioate polymer backbone is attached to a cysteine of the gene editing agent (e.g., first or second gene editing agent) or the targeting agent. In embodiments, the gene editing agent (e.g., first or second gene editing agent) or the targeting agent includes phosphorothioate nucleic acids or phosphorothioate polymer backbones attached to 10%, 25%, 50%, 75%, 90%, 95%, or 100% of the lysines, arginines, cysteines, histidines, or combinations thereof of the gene editing agent (e.g., first or second gene editing agent) or the targeting agent.

As discussed above, the nucleic acids, e.g., the phosphorothioate nucleic acids or phosphorothiate polymer backbones can be attached to the gene editing agent (e.g., first or second gene editing agent) or the targeting agent through a variety of mechanisms. The phosphorothioate nucleic acid or phosphorothioate polymer backbone can be covalently or non-covalently attached to the gene editing agent (e.g., first or second gene editing agent) or the targeting agent. In embodiments, when a plurality of phosphorothioate nucleic acids or phosphorothioate polymer backbones are attached to the gene editing agent (e.g., first or second gene editing agent) or the targeting agent, each of the plurality can be covalently or non-covalently attached to the gene editing agent (e.g., first or second gene editing agent) or the targeting agent. In embodiments, the gene editing agent (e.g., first or second gene editing agent) or the targeting agent includes covalently and non-covalently attached phosphorothioate nucleic acids or phosphorothioate polymer backbones. In embodiments, the gene editing agent (e.g., first or second gene editing agent) or the targeting agent includes covalently attached phosphorothioate nucleic acids or phosphorothioate polymer backbones and does not comprise non-covalently attached phosphorothioate nucleic acids or phosphorothioate polymer backbones. In embodiments, the gene editing agent (e.g., first or second gene editing agent) or the targeting agent includes non-covalently attached phosphorothioate nucleic acids or phosphorothioate polymer backbones and does no comprise covalently attached phosphorothioate nucleic acids or phosphorothioate polymer backbones. Each of the phosphorothioate nucleic acids or phosphorothioate polymer backbones may contain a reactive moiety, e.g., an amino acid reactive moiety or covalent reactive moiety, that facilitates attachment of the phosphorothioate nucleic acid or phosphorothioate polymer backbone to the gene editing agent (e.g., first or second gene editing agent) or the targeting agent. Thus, the phosphorothioate nucleic acids or phosphorothioate polymer backbones can be attached to the gene editing agent (e.g., first or second gene editing agent) or the targeting agent through a reactive moiety.

The complexes provided herein may be made by contacting an unattached gene editing agent (e.g., first or second gene editing agent) or targeting agent with an unattached phosphorothioate nucleic acid or unattached phosphorothioate polymer backbone and allowing the unattached phosphorothioate nucleic acid or unattached phosphorothioate polymer backbone to covalently bind to an amino acid of the unattached gene editing agent (e.g., first or second gene editing agent) or targeting agent thereby attaching and forming the gene editing complex. The use of the term “unattached” as used within the context of making the gene editing complexes is intended to indicate the state of the gene editing agent (e.g., first or second gene editing agent), the targeting agent, phosphorothioate nucleic acid or phosphorothioate polymer backbone prior to attachment and formation of the complex. That is, the term “unattached” indicates that the gene editing agent (e.g., first or second gene editing agent), targeting agent, phosphorothioate nucleic acid or phosphorothioate polymer backbone are free and in their unbound state relative to their associated form within the gene editing complex.

In embodiments, the phosphorothioate nucleic acid or phosphorothioate polymer backbone includes a covalent reactive moiety. As described above, the covalent reactive moiety may reactive with a lysine, arginine, cysteine or histidine of the protein (e.g. with the amino acid side chains). In embodiments, the covalent reactive moiety is reactive with a cysteine. The covalent reactive moiety is a thiol reactive moiety.

In embodiments, the phosphorothioate nucleic acid is a single stranded nucleic acid. In embodiments, the phosphorothioate nucleic acid is a phosphorothioate deoxyribonucleic acid. In embodiments, the phosphorothioate nucleic acid is about 10, 20, 30, 40, 50, 60, 70, 80, 90, 100 or more nucleic acid residues in length. In embodiments, the phosphorothioate nucleic acid is 10, 20, 30, 40, 50, 60, 70, 80, 90, 100 or more nucleic acid residues in length. In embodiments, the phosphorothioate nucleic acid is 10, 20, 30, 40, 50, 60, 70, 80, 90, or 100 nucleic acid residues in length. In embodiments, the phosphorothioate nucleic acid is 10 nucleic acid residues in length. In embodiments, the phosphorothioate nucleic acid is 20 nucleic acid residues in length. In embodiments, the phosphorothioate nucleic acid is 30 nucleic acid residues in length. In embodiments, the phosphorothioate nucleic acid is 40 nucleic acid residues in length. In embodiments, the phosphorothioate nucleic acid is 50 nucleic acid residues in length. In embodiments, the phosphorothioate nucleic acid is 60 nucleic acid residues in length. In embodiments, the phosphorothioate nucleic acid is 70 nucleic acid residues in length. In embodiments, the phosphorothioate nucleic acid is 80 nucleic acid residues in length. In embodiments, the phosphorothioate nucleic acid is 90 nucleic acid residues in length. In embodiments, the phosphorothioate nucleic acid is 100 nucleic acid residues in length.

In embodiments, the phosphorothioate nucleic acid is from about 10 to about 30 nucleic acid residues in length. In embodiments, the phosphorothioate nucleic acid is about 20 nucleic acid residues in length.

In embodiments, the gene editing agent is an RNA-guided DNA endonuclease, a transcription activator-like effector nuclease (TALEN), a zinc finger nuclease, or an Argonaut endonuclease. In embodiments, the gene editing agent is an RNA-guided DNA endonuclease. In embodiments, the gene editing agent is a transcription activator-like effector nuclease (TALEN). In embodiments, the gene editing agent is a zinc finger nuclease. In embodiments, the gene editing agent is an Argonaut endonuclease.

In embodiments, the RNA-guided DNA endonuclease is Cas9, Cpf1 or a Class II CRISPR endonuclease. In embodiments, the RNA-guided DNA endonuclease is Cas9. In embodiments, the RNA-guided DNA endonuclease is Cpf1. In embodiments, the RNA-guided DNA endonuclease is a Class II CRISPR endonuclease. Non-limiting examples of RNA-guided DNA endonuclease include Cas9, GeoCas9, CasX, CasY, Cas14a, Cas12a (Cpf1), Cas9 Nickases, High-Fidelity Cas9, sSpCas9, SpCas9-HF1, HypaCas9, Fok1-Fused dCas9, xCas9, Cas13a, b and d and other CRISPR based editors.

In embodiments, the gene editing agent includes the sequence of SEQ ID NO:2, SEQ ID NO:3, SEQ ID NO:4, SEQ ID NO:5, SEQ ID NO:6, SEQ ID NO:7, SEQ ID NO:18, SEQ ID NO:22, SEQ ID NO:25, or SEQ ID NO:27. In embodiments, the gene editing agent includes the sequence of SEQ ID NO:2. In embodiments, the gene editing agent includes the sequence of SEQ ID NO:3. In embodiments, the gene editing agent includes the sequence of SEQ ID NO:4. In embodiments, the gene editing agent includes the sequence of SEQ ID NO:5. In embodiments, the gene editing agent includes the sequence of SEQ ID NO:6. In embodiments, the gene editing agent includes the sequence of SEQ ID NO: 7. In embodiments, the gene editing agent includes the sequence of SEQ ID NO:18. In embodiments, the gene editing agent includes the sequence of, SEQ ID NO:22. In embodiments, the gene editing agent includes the sequence of SEQ ID NO:25. In embodiments, the gene editing agent includes the sequence of SEQ ID NO:27.

In embodiments, the gene editing agent has the sequence of SEQ ID NO:2, SEQ ID NO:3, SEQ ID NO:4, SEQ ID NO:5, SEQ ID NO:6, SEQ ID NO:7, SEQ ID NO:18, SEQ ID NO:22, SEQ ID NO:25, or SEQ ID NO:27. In embodiments, the gene editing agent has the sequence of SEQ ID NO:2. In embodiments, the gene editing agent has the sequence of SEQ ID NO:3. In embodiments, the gene editing agent has the sequence of SEQ ID NO:4. In embodiments, the gene editing agent has the sequence of SEQ ID NO:5. In embodiments, the gene editing agent has the sequence of SEQ ID NO:6. In embodiments, the gene editing agent has the sequence of SEQ ID NO:7. In embodiments, the gene editing agent has the sequence of SEQ ID NO:18. In embodiments, the gene editing agent has the sequence of, SEQ ID NO:22. In embodiments, the gene editing agent has the sequence of SEQ ID NO:25. In embodiments, the gene editing agent has the sequence of SEQ ID NO:27.

In embodiments, the gene editing agent is encoded by a nucleic acid including the sequence of SEQ ID NO:17, SEQ ID NO:21, SEQ ID NO:24 or SEQ ID NO:26. In embodiments, the gene editing agent is encoded by a nucleic acid including the sequence of SEQ ID NO:17. In embodiments, the gene editing agent is encoded by a nucleic acid including the sequence of SEQ ID NO:21. In embodiments, the gene editing agent is encoded by a nucleic acid including the sequence of SEQ ID NO:24. In embodiments, the gene editing agent is encoded by a nucleic acid including the sequence of SEQ ID NO:26.

In embodiments, the complex forms part of a cell. In embodiments, the cell is a cancer cell or a healthy cell. In embodiments, the cell is a cancer cell. In embodiments, the cell is a healthy cell. In embodiments, the cell is a T cell, a chimeric antigen receptor (CAR) T cell, a natural killer (Nk) cell, a macrophage, a neuronal cell or a hematopoietic stem cell. In embodiments, the cell is a T cell. In embodiments, the cell is a chimeric antigen receptor (CAR) T cell. In embodiments, the cell is a natural killer (Nk) cell. In embodiments, the cell is a macrophage. In embodiments, the cell is a neuronal cell. In embodiments, the cell is a hematopoietic stem cell. In embodiments, the cell is a pancreatic cancer cell or an ovarian cancer cell. In embodiments, the cell is a pancreatic cancer cell. In embodiments, the cell is an ovarian cancer cell.

In embodiments, the complex further includes one or more guide RNAs bound to the gene editing agent. In embodiments, the one or more guide RNA is complementary to one or more target sequences in said cell. In embodiments, the one or more target sequence is a STAT-3 target sequence. In embodiments, the guide RNA includes the sequence of SEQ ID NO:1 (SEQ ID NO:1 ACAATCCGGGCAATCTCCATTGG). In embodiments, the guide RNA includes the sequence of SEQ ID NO:30. In embodiments, the guide RNA includes the sequence of SEQ ID NO:37. In embodiments, the guide RNA includes the sequence of SEQ ID NO:38.

In embodiments, the one or more target sequence is a Programmed cell death protein 1 (PDCD1) target sequence. In embodiments, the guide RNA includes the sequence of SEQ ID NO:31. In embodiments, the guide RNA is the sequence of SEQ ID NO:31.

A “PDCD1” or “PDCD1 protein” as referred to herein includes any of the recombinant or naturally-occurring forms of the Programmed cell death protein 1 (PDCD1) or variants or homologs thereof that maintain PDCD1 activity (e.g. within at least 50%, 80%, 90%, 95%, 96%, 97%, 98%, 99% or 100% activity compared to PDCD1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PDCD1 protein. In embodiments, the PDCD1 protein is substantially identical to the protein identified by the NCBI reference number NP_005009 or a variant or homolog having substantial identity thereto. In embodiments, the PDCD1 protein has at least 75% sequence identity to the amino acid sequence of the protein identified by the NCBI reference number NP_005009. In embodiments, the PDCD1 protein has at least 80% sequence identity to the amino acid sequence of the protein identified by the NCBI reference number NP_005009. In embodiments, the PDCD1 protein has at least 85% sequence identity to the amino acid sequence of the protein identified by the NCBI reference number NP_005009. In embodiments, the PDCD1 protein has at least 90% sequence identity to the amino acid sequence of the protein identified by the NCBI reference number NP_005009. In embodiments, the PDCD1 protein has at least 95% sequence identity to the amino acid sequence of the protein identified by the NCBI reference number NP_005009. In embodiments, the PDCD1 protein has at least 98% sequence identity to the amino acid sequence of the protein identified by the NCBI reference number NP_005009. In embodiments, the PDCD1 protein has at least 99% sequence identity to the amino acid sequence of the protein identified by the NCBI reference number NP_005009. In embodiments, the PDCD1 protein has 100% sequence identity to the amino acid sequence of the protein identified by the NCBI reference number NP_005009.

In embodiments, the one or more target sequence is a Programmed cell death protein 1 (PDCD2) target sequence. In embodiments, the guide RNA includes the sequence of SEQ ID NO:32. In embodiments, the guide RNA is the sequence of SEQ ID NO:32.

A “PDCD2” or “PDCD2 protein” as referred to herein includes any of the recombinant or naturally-occurring forms of the Programmed cell death protein 2 (PDCD2) or variants or homologs thereof that maintain PDCD2 activity (e.g. within at least 50%, 80%, 90%, 95%, 96%, 97%, 98%, 99% or 100% activity compared to PDCD2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PDCD2 protein. In embodiments, the PDCD2 protein is substantially identical to the protein identified by the UniProt reference number Q16342 or a variant or homolog having substantial identity thereto. In embodiments, the PDCD2 protein has at least 75% sequence identity to the amino acid sequence of the protein identified by the UniProt reference number Q16342. In embodiments, the PDCD2 protein has at least 80% sequence identity to the amino acid sequence of the protein identified by the UniProt reference number Q16342. In embodiments, the PDCD2 protein has at least 85% sequence identity to the amino acid sequence of the protein identified by the UniProt reference number Q16342. In embodiments, the PDCD2 protein has at least 90% sequence identity to the amino acid sequence of the protein identified by the UniProt reference number Q16342. In embodiments, the PDCD2 protein has at least 95% sequence identity to the amino acid sequence of the protein identified by the UniProt reference number Q16342. In embodiments, the PDCD2 protein has at least 98% sequence identity to the amino acid sequence of the protein identified by the UniProt reference number Q16342. In embodiments, the PDCD2 protein has at least 99% sequence identity to the amino acid sequence of the protein identified by the UniProt reference number Q16342. In embodiments, the PDCD2 protein has 100% sequence identity to the amino acid sequence of the protein identified by the UniProt reference number Q16342.

In embodiments, the one or more target sequence is a Tet methylcytosine dioxygenase 2 (TET2) target sequence. In embodiments, the guide RNA includes the sequence of SEQ ID NO:33. In embodiments, the guide RNA is the sequence of SEQ ID NO:33.

In embodiments, the one or more target sequence is a PARG Poly (ADP-ribose) glycohydrolase target sequence. In embodiments, the guide RNA includes the sequence of SEQ ID NO:34. In embodiments, the guide RNA includes the sequence of SEQ ID NO:34.

A “PARG” or “PARG protein” as referred to herein includes any of the recombinant or naturally-occurring forms of the PARG Poly (ADP-ribose) glycohydrolase or variants or homologs thereof that maintain PARG activity (e.g. within at least 50%, 80%, 90%, 95%, 96%, 97%, 98%, 99% or 100% activity compared to PARG). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PARG protein. In embodiments, the PARG protein is substantially identical to the protein identified by the UniProt reference number

Q86W56 or a variant or homolog having substantial identity thereto. In embodiments, the PARG protein has at least 75% sequence identity to the amino acid sequence of the protein identified by the UniProt reference number Q86W56. In embodiments, the PARG protein has at least 80% sequence identity to the amino acid sequence of the protein identified by the UniProt reference number Q86W56. In embodiments, the PARG protein has at least 85% sequence identity to the amino acid sequence of the protein identified by the UniProt reference number Q86W56. In embodiments, the PARG protein has at least 90% sequence identity to the amino acid sequence of the protein identified by the UniProt reference number Q86W56. In embodiments, the PARG protein has at least 95% sequence identity to the amino acid sequence of the protein identified by the UniProt reference number Q86W56. In embodiments, the PARG protein has at least 98% sequence identity to the amino acid sequence of the protein identified by the UniProt reference number Q86W56. In embodiments, the PARG protein has at least 99% sequence identity to the amino acid sequence of the protein identified by the UniProt reference number Q86W56.

In embodiments, the one or more target sequence is a T cell receptor-alpha (TCR-a) target sequence. In embodiments, the guide RNA includes the sequence of SEQ ID NO:35. In embodiments, the guide RNA is the sequence of SEQ ID NO:35.

In embodiments, the one or more target sequence is a T cell receptor-beta (TCR-b) target sequence. In embodiments, the guide RNA includes the sequence of SEQ ID NO:36. In embodiments, the guide RNA is the sequence of SEQ ID NO:36.

In embodiments, the one or more target sequence is a Vascular endothelial growth factor A-alpha (VEGFA-a) target sequence. In embodiments, the guide RNA includes the sequence of SEQ ID NO:39. In embodiments, the guide RNA is the sequence of SEQ ID NO:39.

In embodiments, the one or more target sequence is a Vascular endothelial growth factor A-beta (VEGFA-b) target sequence. In embodiments, the guide RNA includes the sequence of SEQ ID NO:40. In embodiments, the guide RNA is the sequence of SEQ ID NO:40.

In embodiments, the guide RNA includes the sequence of SEQ ID NO:1, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

### Editing Complexes Including a Second Gene Editing Agent or A Targeting Agent

In an aspect, a complex for delivering a gene editing agent to a cell is provided. The complex includes (i) a double-stranded phosphorothioate oligonucleotide; (ii) a first gene editing agent covalently bound to a first phosphorothioate nucleic acid through a first chemical linker; and (ii) a second gene editing agent covalently bound to a second phosphorothioate nucleic acid through a second chemical linker; wherein at least a portion of the first phosphorothioate nucleic acid and a portion of the second phosphorothioate nucleic acid are complementary to each other and wherein at least a portion of the first phosphorothioate nucleic acid is hybridized to at least a portion of the second phosphorothioate nucleic acid thereby forming the double-stranded phosphorothioate oligonucleotide.

In another aspect, a complex for delivering a gene editing agent to a cell is provided. The complex includes (i) a double-stranded phosphorothioate oligonucleotide; (ii) a gene editing agent covalently bound to a first phosphorothioate nucleic acid through a first chemical linker; and (ii) a targeting agent covalently bound to a second phosphorothioate nucleic acid through a second chemical linker, wherein at least a portion of the first phosphorothioate nucleic acid and a portion of the second phosphorothioate nucleic acid are complementary to each other. And wherein at least a portion of the first phosphorothioate nucleic acid is hybridized to at least a portion of the second phosphorothioate nucleic acid thereby forming the double-stranded phosphorothioate oligonucleotide.

The term “at least a portion” when used in the context of complementarity between or hybridization of two nucleic acid sequences refers to its ordinary meaning in the biological arts. Where “at least a portion” of nucleotides of a first and a second nucleic acid sequence are complementary to each other, at least one (e.g., 2, 3, 4, 5, 6, 7, 8, 9, 10, 15, 20) nucleotide of the first nucleic acid forms a proper base pair (i.e., adenine with thymine and guanine with cytosine, not a mismatch) with a nucleotide of the second nucleic acid.

In embodiments, 2-5 nucleotides of the first phosphorothioate nucleic acid are complementary to 2-5 nucleotides of the second phosphorothioate nucleic acid. In embodiments, 2-5 contiguous nucleotides of the first phosphorothioate nucleic acid are complementary to 2-5 contiguous nucleotides of the second phosphorothioate nucleic acid. In embodiments, 2-6 nucleotides of the first phosphorothioate nucleic acid are complementary to 2-6 nucleotides of the second phosphorothioate nucleic acid. In embodiments, 2-6 contiguous nucleotides of the first phosphorothioate nucleic acid are complementary to 2-6 contiguous nucleotides of the second phosphorothioate nucleic acid. In embodiments, 2-7 nucleotides of the first phosphorothioate nucleic acid are complementary to 2-7 nucleotides of the second phosphorothioate nucleic acid. In embodiments, 2-7 contiguous nucleotides of the first phosphorothioate nucleic acid are complementary to 2-7 contiguous nucleotides of the second phosphorothioate nucleic acid. In embodiments, 2-8 nucleotides of the first phosphorothioate nucleic acid are complementary to 2-8 nucleotides of the second phosphorothioate nucleic acid. In embodiments, 2-8 contiguous nucleotides of the first phosphorothioate nucleic acid are complementary to 2-8 contiguous nucleotides of the second phosphorothioate nucleic acid. In embodiments, 2-9 nucleotides of the first phosphorothioate nucleic acid are complementary to 2-9 nucleotides of the second phosphorothioate nucleic acid. In embodiments, 2-9 contiguous nucleotides of the first phosphorothioate nucleic acid are complementary to 2-9 contiguous nucleotides of the second phosphorothioate nucleic acid. In embodiments, 2-10 nucleotides of the first phosphorothioate nucleic acid are complementary to 2-10 nucleotides of the second phosphorothioate nucleic acid. In embodiments, 2-10 contiguous nucleotides of the first phosphorothioate nucleic acid are complementary to 2-10 contiguous nucleotides of the second phosphorothioate nucleic acid.

In embodiments, 2-11 nucleotides of the first phosphorothioate nucleic acid are complementary to 2-11 nucleotides of the second phosphorothioate nucleic acid. In embodiments, 2-11 contiguous nucleotides of the first phosphorothioate nucleic acid are complementary to 2-11 contiguous nucleotides of the second phosphorothioate nucleic acid. In embodiments, 2-12 nucleotides of the first phosphorothioate nucleic acid are complementary to 2-12 nucleotides of the second phosphorothioate nucleic acid. In embodiments, 2-12 contiguous nucleotides of the first phosphorothioate nucleic acid are complementary to 2-12 contiguous nucleotides of the second phosphorothioate nucleic acid. In embodiments, 2-13 nucleotides of the first phosphorothioate nucleic acid are complementary to 2-13 nucleotides of the second phosphorothioate nucleic acid. In embodiments, 2-13 contiguous nucleotides of the first phosphorothioate nucleic acid are complementary to 2-13 contiguous nucleotides of the second phosphorothioate nucleic acid. In embodiments, 2-14 nucleotides of the first phosphorothioate nucleic acid are complementary to 2-14 nucleotides of the second phosphorothioate nucleic acid. In embodiments, 2-14 contiguous nucleotides of the first phosphorothioate nucleic acid are complementary to 2-14 contiguous nucleotides of the second phosphorothioate nucleic acid. In embodiments, 2-15 nucleotides of the first phosphorothioate nucleic acid are complementary to 2-15 nucleotides of the second phosphorothioate nucleic acid. In embodiments, 2-15 contiguous nucleotides of the first phosphorothioate nucleic acid are complementary to 2-15 contiguous nucleotides of the second phosphorothioate nucleic acid. In embodiments, 2-16 nucleotides of the first phosphorothioate nucleic acid are complementary to 2-16 nucleotides of the second phosphorothioate nucleic acid. In embodiments, 2-16 contiguous nucleotides of the first phosphorothioate nucleic acid are complementary to 2-16 contiguous nucleotides of the second phosphorothioate nucleic acid.

In embodiments, 2-17 nucleotides of the first phosphorothioate nucleic acid are complementary to 2-17 nucleotides of the second phosphorothioate nucleic acid. In embodiments, 2-17 contiguous nucleotides of the first phosphorothioate nucleic acid are complementary to 2-17 contiguous nucleotides of the second phosphorothioate nucleic acid. In embodiments, 2-18 nucleotides of the first phosphorothioate nucleic acid are complementary to 2-18 nucleotides of the second phosphorothioate nucleic acid. In embodiments, 2-18 contiguous nucleotides of the first phosphorothioate nucleic acid are complementary to 2-18 contiguous nucleotides of the second phosphorothioate nucleic acid. In embodiments, 2-19 nucleotides of the first phosphorothioate nucleic acid are complementary to 2-19 nucleotides of the second phosphorothioate nucleic acid. In embodiments, 2-19 contiguous nucleotides of the first phosphorothioate nucleic acid are complementary to 2-19 contiguous nucleotides of the second phosphorothioate nucleic acid. In embodiments, 2-20 nucleotides of the first phosphorothioate nucleic acid are complementary to 2-20 nucleotides of the second phosphorothioate nucleic acid. In embodiments, 2-20 contiguous nucleotides of the first phosphorothioate nucleic acid are complementary to 2-20 contiguous nucleotides of the second phosphorothioate nucleic acid.

In embodiments, 20 nucleotides of the first phosphorothioate nucleic acid are complementary to 20 nucleotides of the second phosphorothioate nucleic acid. In embodiments, 22 contiguous nucleotides of the first phosphorothioate nucleic acid are complementary to 22 contiguous nucleotides of the second phosphorothioate nucleic acid.

In embodiments, the first phosphorothioate nucleic acid and the second phosphorothioate nucleic acid are 70%, 75%, 80%, 85%, 90%, 95%, 98%, 99% or 100% complementary to each other.

Where “at least a portion” of the first phosphorothioate nucleic acid is hybridized to at least a portion of the second phosphorothioate nucleic acid at least one (e.g., 2, 3, 4, 5, 6, 7, 8, 9, 10, 15, 20) nucleotide of the first nucleic acid forms a proper base pair (i.e., adenine with thymine and guanine with cytosine, not a mismatch) with a nucleotide of the second nucleic acid thereby forming a double stranded nucleic acid molecule. In embodiments, the portion includes 2, 3, 4, 5, 6, 7, 8, 9, 10, 11, 12, 13, 14, 15, 16, 17, 18, 19, 20, 21, 22, 23, 24, 25, 26, 27, 28, 29, 30 or more base pairs.

In embodiments, 2-5 nucleotides of the first phosphorothioate nucleic acid are hybridized to 2-5 nucleotides of the second phosphorothioate nucleic acid. In embodiments, 2-5 contiguous nucleotides of the first phosphorothioate nucleic acid are complementary to 2-5 contiguous nucleotides of the second phosphorothioate nucleic acid. In embodiments, 2-6 nucleotides of the first phosphorothioate nucleic acid are hybridized to 2-6 nucleotides of the second phosphorothioate nucleic acid. In embodiments, 2-6 contiguous nucleotides of the first phosphorothioate nucleic acid are hybridized to 2-6 contiguous nucleotides of the second phosphorothioate nucleic acid. In embodiments, 2-7 nucleotides of the first phosphorothioate nucleic acid are hybridized to 2-7 nucleotides of the second phosphorothioate nucleic acid. In embodiments, 2-7 contiguous nucleotides of the first phosphorothioate nucleic acid are hybridized to 2-7 contiguous nucleotides of the second phosphorothioate nucleic acid. In embodiments, 2-8 nucleotides of the first phosphorothioate nucleic acid are hybridized to 2-8 nucleotides of the second phosphorothioate nucleic acid. In embodiments, 2-8 contiguous nucleotides of the first phosphorothioate nucleic acid are hybridized to 2-8 contiguous nucleotides of the second phosphorothioate nucleic acid. In embodiments, 2-9 nucleotides of the first phosphorothioate nucleic acid are hybridized to 2-9 nucleotides of the second phosphorothioate nucleic acid. In embodiments, 2-9 contiguous nucleotides of the first phosphorothioate nucleic acid are hybridized to 2-9 contiguous nucleotides of the second phosphorothioate nucleic acid. In embodiments, 2-10 nucleotides of the first phosphorothioate nucleic acid are hybridized to 2-10 nucleotides of the second phosphorothioate nucleic acid. In embodiments, 2-10 contiguous nucleotides of the first phosphorothioate nucleic acid are hybridized to 2-10 contiguous nucleotides of the second phosphorothioate nucleic acid.

In embodiments, 2-11 nucleotides of the first phosphorothioate nucleic acid are hybridized to 2-11 nucleotides of the second phosphorothioate nucleic acid. In embodiments, 2-11 contiguous nucleotides of the first phosphorothioate nucleic acid are hybridized to 2-11 contiguous nucleotides of the second phosphorothioate nucleic acid. In embodiments, 2-12 nucleotides of the first phosphorothioate nucleic acid are hybridized to 2-12 nucleotides of the second phosphorothioate nucleic acid. In embodiments, 2-12 contiguous nucleotides of the first phosphorothioate nucleic acid are hybridized to 2-12 contiguous nucleotides of the second phosphorothioate nucleic acid. In embodiments, 2-13 nucleotides of the first phosphorothioate nucleic acid are hybridized to 2-13 nucleotides of the second phosphorothioate nucleic acid. In embodiments, 2-13 contiguous nucleotides of the first phosphorothioate nucleic acid are hybridized to 2-13 contiguous nucleotides of the second phosphorothioate nucleic acid. In embodiments, 2-14 nucleotides of the first phosphorothioate nucleic acid are hybridized to 2-14 nucleotides of the second phosphorothioate nucleic acid. In embodiments, 2-14 contiguous nucleotides of the first phosphorothioate nucleic acid are hybridized to 2-14 contiguous nucleotides of the second phosphorothioate nucleic acid. In embodiments, 2-15 nucleotides of the first phosphorothioate nucleic acid are hybridized to 2-15 nucleotides of the second phosphorothioate nucleic acid. In embodiments, 2-15 contiguous nucleotides of the first phosphorothioate nucleic acid are hybridized to 2-15 contiguous nucleotides of the second phosphorothioate nucleic acid. In embodiments, 2-16 nucleotides of the first phosphorothioate nucleic acid are hybridized to 2-16 nucleotides of the second phosphorothioate nucleic acid. In embodiments, 2-16 contiguous nucleotides of the first phosphorothioate nucleic acid are hybridized to 2-16 contiguous nucleotides of the second phosphorothioate nucleic acid.

In embodiments, 2-17 nucleotides of the first phosphorothioate nucleic acid are hybridized to 2-17 nucleotides of the second phosphorothioate nucleic acid. In embodiments, 2-17 contiguous nucleotides of the first phosphorothioate nucleic acid are hybridized to 2-17 contiguous nucleotides of the second phosphorothioate nucleic acid. In embodiments, 2-18 nucleotides of the first phosphorothioate nucleic acid are hybridized to 2-18 nucleotides of the second phosphorothioate nucleic acid. In embodiments, 2-18 contiguous nucleotides of the first phosphorothioate nucleic acid are hybridized to 2-18 contiguous nucleotides of the second phosphorothioate nucleic acid. In embodiments, 2-19 nucleotides of the first phosphorothioate nucleic acid are hybridized to 2-19 nucleotides of the second phosphorothioate nucleic acid. In embodiments, 2-19 contiguous nucleotides of the first phosphorothioate nucleic acid are hybridized to 2-19 contiguous nucleotides of the second phosphorothioate nucleic acid. In embodiments, 2-20 nucleotides of the first phosphorothioate nucleic acid are hybridized to 2-20 nucleotides of the second phosphorothioate nucleic acid. In embodiments, 2-20 contiguous nucleotides of the first phosphorothioate nucleic acid are hybridized to 2-20 contiguous nucleotides of the second phosphorothioate nucleic acid.

In embodiments, 20 nucleotides of the first phosphorothioate nucleic acid are hybridized to 20 nucleotides of the second phosphorothioate nucleic acid. In embodiments, 22 contiguous nucleotides of the first phosphorothioate nucleic acid are hybridized to 22 contiguous nucleotides of the second phosphorothioate nucleic acid.

Any of the gene editing agents described above including embodiments thereof may be used for the gene editing complexes described in this section. Thus, in embodiments, the first gene editing agent and the second gene editing agent are independently an RNA-guided DNA endonuclease, a transcription activator-like effector nuclease (TALEN), a zinc finger nuclease, or an Argonaut endonuclease. In embodiments, the first gene editing agent and the second gene editing agent are independently Cas9, Cpf1 or a Class II CRISPR endonuclease. In embodiments, the first gene editing agent is a first Cas9 and the second gene editing agent is a second Cas9. In embodiments, the first gene editing agent and the second gene editing agent are independently nuclease-deficient. In embodiments, the first gene editing agent includes a first cysteine and the first phosphorothioate nucleic acid includes a first thiol moiety covalently bound to the first gene editing agent through a disulfide linkage between the first cysteine and the first thiol moiety.

In embodiments, the second gene editing agent includes a second cysteine and the second phosphorothioate nucleic acid includes a second thiol moiety covalently bound to the second gene editing agent through a disulfide linkage between the second cysteine and the second thiol moiety. In embodiments, the first phosphorothioate nucleic acid is bound to the C-terminus of the first gene editing agent. In embodiments, the second phosphorothioate nucleic acid is bound to the C-terminus of the second gene editing agent.

Any of the chemical linkers described above including embodiments thereof may be used for the gene editing complexes described in this section. Thus, in embodiments, the first chemical linker and the second chemical linker are independently a pH-sensitive linker. In embodiments, the first chemical linker and the second chemical linker are independently a thioester linker.

Any of the phosphorothioate nucleic acids described above including embodiments thereof may be used for the gene editing complexes described in this section. In embodiments, the first phosphorothioate nucleic acid and the second phosphorothioate nucleic acid are independently a phosphorothioate deoxyribonucleic acid or a phosphorothioate ribonucleic acid. In embodiments, the first phosphorothioate nucleic acid and the second phosphorothioate nucleic acid are independently about 10, 20, 30, 40, 50, 60, 70, 80, 90, 100 or more nucleic acid residues in length. In embodiments, the first phosphorothioate nucleic acid and the second phosphorothioate nucleic acid are independently from about 10 to about 30 nucleic acid residues in length. In embodiments, the one or more guide RNAs are complementary to one or more target sequences in the cell.

In embodiments, the complex further includes one or more guide RNA bound to the first gene editing agent or the second gene editing agent. Any of the guide RNAs described above including embodiments thereof may be used for the gene editing complexes described in this section. In embodiments, the guide RNA includes the sequence of SEQ ID NO:1, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

In embodiments, the complex forms part of a cell. Any embodiments described for the gene editing complexes above are applicable and envisioned of the gene editing complexes described in this section. Thus, in embodiments, the cell is a cancer cell or a healthy cell. In embodiments, the cell is a T cell, a chimeric antigen receptor (CAR) T cell, a natural killer (Nk) cell, a macrophage, a neuronal cell or a hematopoietic stem cell. In embodiments, the cell is a pancreatic cancer cell or an ovarian cancer cell.

In embodiments, the one or more guide RNA is complementary to one or more target sequence in the cell. Any of the target sequences described above including embodiments thereof may be used for the gene editing complexes described in this section. In embodiments, the one or more target sequence is a STAT-3 target sequence, a Programmed cell death protein 1 (PDCD1) target sequence, a Programmed cell death protein 1 (PDCD2) target sequence, a Tet methylcytosine dioxygenase 2 (TET2) target sequence, a PARG Poly (ADP-ribose) glycohydrolase target sequence, a T cell receptor-alpha (TCR-a) target sequence, a T cell receptor-beta (TCR-b) target sequence, a Vascular endothelial growth factor A-alpha (VEGFA-a) target sequence or a Vascular endothelial growth factor A-beta (VEGFA-b) target sequence.

In embodiments, the one or more guide RNA includes the sequence of SEQ ID NO:1, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

In another aspect, a complex for delivering a gene editing agent to a cell is provided. The complex includes (i) a double-stranded phosphorothioate oligonucleotide; (ii) a gene editing agent covalently bound to a first phosphorothioate nucleic acid through a first chemical linker; and (ii) a targeting agent covalently bound to a second phosphorothioate nucleic acid through a second chemical linker, wherein at least a portion of the first phosphorothioate nucleic acid and a portion of the second phosphorothioate nucleic acid are complementary to each other. And wherein at least a portion of the first phosphorothioate nucleic acid is hybridized to at least a portion of the second phosphorothioate nucleic acid thereby forming the double-stranded phosphorothioate oligonucleotide.

Any of the gene editing agent described above including embodiments thereof may be used for the gene editing complexes described in this section. In embodiments, the gene editing agent is an RNA-guided DNA endonuclease, a transcription activator-like effector nuclease (TALEN), a zinc finger nuclease, or an Argonaut endonuclease. In embodiments, the gene editing agent is Cas9, Cpf1 or a Class II CRISPR endonuclease. In embodiments, the gene editing agent is Cas9.

In embodiments, the targeting agent is a protein or a nucleic acid. In embodiments, the targeting agent is an antibody. In embodiments, the targeting agent is a cancer-specific antibody. In embodiments, the antibody is an anti-HER2 antibody. Non limiting examples of antibodies contemplated for the methods and compositions provide herein include, Anti-Her2 (Margetuximab, trastuzumab deruxtecan, Cemiplimab, Ado-trastuzumab emtansine, Pertuzumab, Trastuzumab), Anti-PD-1 (Sintilimab, Toripalimab, Retifanlimab, Balstilimab, Dostarlimab, Nivolumab, Pembrolizumab), Anti-PD-L1 (Durvalumab, Avelumab, Atezolizumab), Anti-CTLA-4 (Ipilimumab, Anti-IL-2R (Basiliximab, Daclizumab), Anti-B7-H3 (Omburtamab), Anti-tissue factor (Tisotumab vedotin), Anti-EGFR (Amivantamab, Necitumumab, Panitumumab, Cetuximab), Anti-c-MET (Amivantamab, Anti-CD20 (Ublituximab, Ocrelizumab, Obinutuzumab, Ofatumumab, Tositumomab-I131, Ibritumomab tiuxetan, Rituximab), Anti-IFNAR1 (Anifrolumab), Anti-CD19 (Loncastuximab tesirine, Tafasitamab, Inebilizumab, Blinatumomab, Anti-CD3 (Teplizumab, Blinatumomab, Catumaxomab, Muromonab-CD3)

Anti-CD25 (Inolimomab), Anti-EpCAM (Oportuzumab monatox, Catumaxomab, Edrecolomab), Anti-CD52 (Alemtuzumab), Anti-CD33 (Gemtuzumab ozogamicin), Anti-GD2 (Naxitamab, Dinutuximab), Anti-BCMA (Belantamab mafodotin), Anti-IL-6R (Spartalizumab, Sarilumab, Tocilizumab), Anti-CD30 (Brentuximab vedotin), Anti-IL-4Ra (Dupilumab), Anti-TROP-2 (Sacituzumab govitecan), Anti-IGF-1R (Teprotumumab), Anti-CD38 (Isatuximab, Daratumumab), Anti-CGRP receptor, Anti-Nectin-4 (Enfortumab vedotin), Anti-P-selectin (Crizanlizumab), Anti-CCR4 (Mogamulizumab), Anti-VEGF (Brolucizumab, Ranibizumab, Bevacizumab), Anti-VEGFR2 (Ramucirumab), Anti-CD79b (Polatuzumab vedotin), Anti-CD22 (Moxetumomab pasudotox, Inotuzumab ozogamicin), Anti-CD4 (Ibalizumab), Anti-IL-5Rα (Benralizumab), Anti-IL-17R (Brodalumab), Anti-PDGRFα (Olaratumab), Anti-SLAMF7 (Elotuzumab), Anti-α4β7 integrin (Vedolizumab), Anti-Muc1, Anti-CD44, Anti-CD133, Anti-CD5, Anti-CD7, Anti-CD70, Anti-IL13Rα2, Anti-PSCA, Anti-PSMA, Anti-GPC3, Anti-FAP, Anti-CEA, Anti-CD34, Anti-CD123, Anti-CD32, Anti-CD79a, Anti-CD96, Anti-CD123, Anti-ROR1, Anti-MESO antigen receptor, Anti-TIM3, Anti-Src, Anti-Jak1, Anti-Jak2, Anti-c-Kit, Anti-CD36, Anti-MDR1, Anti-FGFR2, Anti-FGFR3 and Anti-FLT3.

In embodiments, the gene editing agent is nuclease-deficient. In embodiments, the gene editing agent includes a first cysteine and the first phosphorothioate nucleic acid includes a first thiol moiety covalently bound to the gene editing agent through a disulfide linkage between the first cysteine and the first thiol moiety. In embodiments, the targeting agent includes a second cysteine and the second phosphorothioate nucleic acid includes a second thiol moiety covalently bound to the targeting agent through a disulfide linkage between the second cysteine and the second thiol moiety. In embodiments, the first phosphorothioate nucleic acid is bound to the C-terminus of the gene editing agent. In embodiments, the second phosphorothioate nucleic acid is independently attached to a lysine, arginine, cysteine, or histidine of the targeting agent.

In embodiments, the first chemical linker and the second chemical linker are independently a pH-sensitive linker. Any of the chemical linkers described above including embodiments thereof may be used for the gene editing complexes described in this section. In embodiments, the first chemical linker and the second chemical linker are independently a thioester linker.

Any of the phosphorothioate nucleic acids described above including embodiments thereof may be used for the gene editing complexes described in this section. In embodiments, the first phosphorothioate nucleic acid and the second phosphorothioate nucleic acid are independently a phosphorothioate deoxyribonucleic acid or a phosphorothioate ribonucleic acid. In embodiments, the first phosphorothioate nucleic acid and the second phosphorothioate nucleic acid are independently about 10, 20, 30, 40, 50, 60, 70, 80, 90, 100 or more nucleic acid residues in length. In embodiments, the first phosphorothioate nucleic acid and the second phosphorothioate nucleic acid are independently from about 10 to about 30 nucleic acid residues in length. In embodiments, the complex further includes one or more guide RNA bound to the gene editing agent. Any of the guide RNAs described above including embodiments thereof may be used for the gene editing complexes described in this section.

In embodiments, the complex forms part of a cell. Any embodiments described for the gene editing complexes above are applicable and envisioned of the gene editing complexes described in this section. Thus, in embodiments, the cell is a cancer cell or a healthy cell. In embodiments, the cell is a T cell, a chimeric antigen receptor (CAR) T cell, a natural killer (Nk) cell, a macrophage, a neuronal cell or a hematopoietic stem cell. In embodiments, the cell is a pancreatic cancer cell or an ovarian cancer cell.

In embodiments, the one or more guide RNA is complementary to one or more target sequence in the cell. Any of the target sequences described above including embodiments thereof may be used for the gene editing complexes described in this section. In embodiments, the one or more target sequence is a STAT-3 target sequence, a Programmed cell death protein 1 (PDCD1) target sequence, a Programmed cell death protein 1 (PDCD2) target sequence, a Tet methylcytosine dioxygenase 2 (TET2) target sequence, a PARG Poly (ADP-ribose) glycohydrolase target sequence, a T cell receptor-alpha (TCR-a) target sequence, a T cell receptor-beta (TCR-b) target sequence, a Vascular endothelial growth factor A-alpha (VEGFA-a) target sequence or a Vascular endothelial growth factor A-beta (VEGFA-b) target sequence.

In embodiments, the one or more guide RNA includes the sequence of SEQ ID NO:1, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

In an aspect, a pharmaceutical composition is provided. The pharmaceutical composition includes a pharmaceutical excipient and a complex as provided herein including embodiments thereof.

The gene editing complexes provided herein including embodiments thereof may be delivered into a cell through attachment to one or more phosphorothioate nucleic acid. The gene editing agents provided herein may be non-cell penetrating proteins, which upon attachment to the phosphorothioate nucleic acid provided herein is internalized into a cell. In embodiments, the gene editing agent is cell penetrating in the presence of the phosphorothioate nucleic acid. In embodiments, the gene editing agent is non-cell penetrating in the absence of the phosphorothioate nucleic acid. In embodiments, the amount of gene editing agent internalized by a cell in the presence of the phosphorothioate nucleic acid is increased relative to the absence of the phosphorothioate nucleic acid. In embodiments, the amount of gene editing agent internalized by a cell in the presence of the phosphorothioate nucleic acid is increased 2, 3, 4, 5, 6, 7, 8, 9, 10, 100, 10000, 10,000-fold relative to the absence of the phosphorothioate nucleic acid. In embodiments, the gene editing agent is not internalized by a cell at detectable amounts in the absence of the phosphorothioate nucleic acid.

In an aspect, a method of delivering a gene editing agent to a cell is provided. The method includes, contacting a cell with a complex as provided herein including embodiments thereof thereby delivering the gene editing agent to the cell. In embodiments, the contacting occurs in vitro or in vivo. In embodiments, the cell is a T cell or a stem cell. In embodiments, the cell is a pluripotent stem cell, an immune cell, a cancer cell, or a neuron. In embodiments, the cell is T cell (e.g., a chimeric antigen receptor T cell), a natural killer cell, a macrophage, or a B cell. In embodiments, the cell is a cancer cell it may be a adrenal gland, ampulla of vater, biliary track, bladder/urinary tract, bone, bowel, breast, CNS/brain, cervix, esophagus/stomach, eye, head and neck, kidney, liver, lung, lymphoid, myeloid, ovary/fallopian tube, pancreas, peripheral nervous system, pleura, prostate, skin, soft tissue, testis, thymus, thyroid, uterus or vulva/vagina cancer cell.

### EXAMPLES

### Example 1

The method provided herein delivers Cas9/guide RNA into T cells, and other cells, with high efficiency (greater than 90%), which is followed by high efficiency in deletion (equal or more than 90%) of the intended gene(s).

Cas9 enzyme was modified with phosphorothioated (PS) ssDNA-oligo including a linker, followed by mixing with guide RNA. The modified Cas9/guide RNA retained efficient enzymatic activities.

The modified Cas9/guide RNA mixture, when added to cultured tumor cells, fresh mouse T cells, primary human T cells, human CAR-T cells, penetrated these cells very efficiently. Internalized modified Cas9/guide RNA deleted targeted gene effectively.

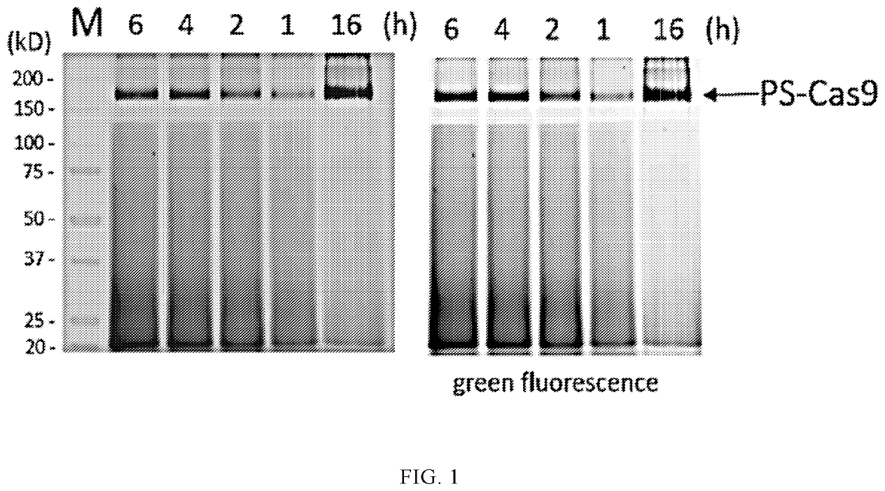

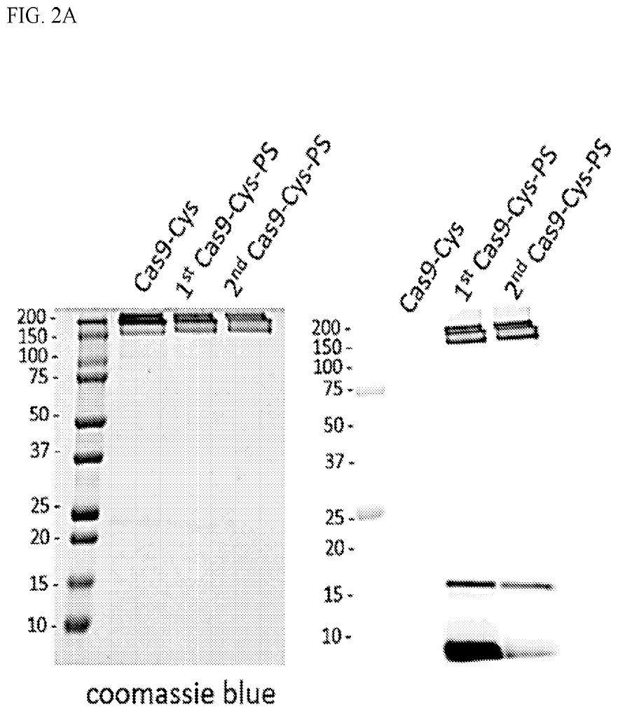

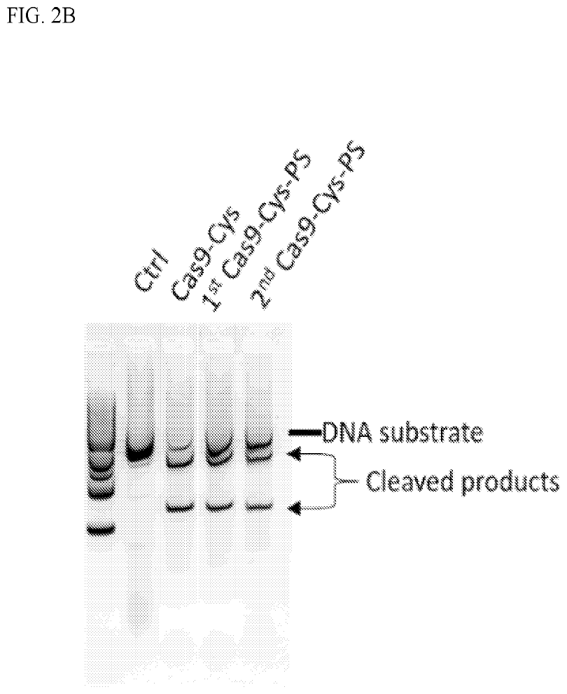

Two attachment chemistries have been tested thus far. The first was Cas9-phosphorothioate-oligo (PS) conjugation through primary amine-aldehyde cross-linking (FIG. 1). Primary amine bond in Cas9 protein was used to conjugate aldehyde-phosphorothioated-ssDNA-oligo. The second attachment chemistry was attachment of the PS-oligo at defined site in Cas9 enzyme. Results showed that attachment on cysteine at C terminal of Cas9 retained full/close to full enzymatic activities, as well as the binding of guide RNA (FIG. 2). The attached PS ssDNA-oligo was detached once the modified enzyme is inside the cells. This approach may also enable attachment of two or more PS ssDNA-oligos at defined sites of Cas9.

In embodiments, the chemical linker may be PS (phosphorothioate nucleic acid)-cysteine bond which can be cleaved either through thiol reductase (disulfide bond) or endosomal/lysosomal cysteine proteases so cysteine linker. Thus, the linker may be used for PS conjugation through disulfide or the any other type of covalent bond. Maleimide-based linkers for cysteine conjugation (thioester bond) may be used: including, but not limited to, maleimidocaproyl (mc) and maleimidomethyl cyclohexane-1-carboxylate (mcc) based linkers, such as mc-Val-Cit- or mc-Val-Ala-linker. The thioester bond can be released by thioesterase or cathepsin B (a protease) once Cas9-PS conjugate penetrates into the cells. Phenylalanine-lysine (Phe-Lys) dipeptide linker with a p-aminobenzoyloxy (PABO) spacer is a cleavable linker that may be used for PS conjugation. PS conjugate can be released after the linker is cleaved by cathepsin B protease.

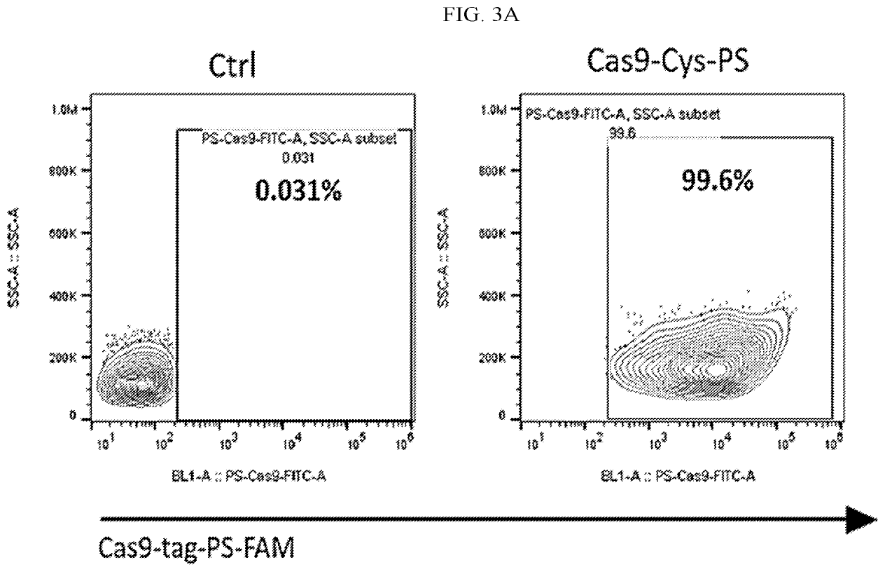

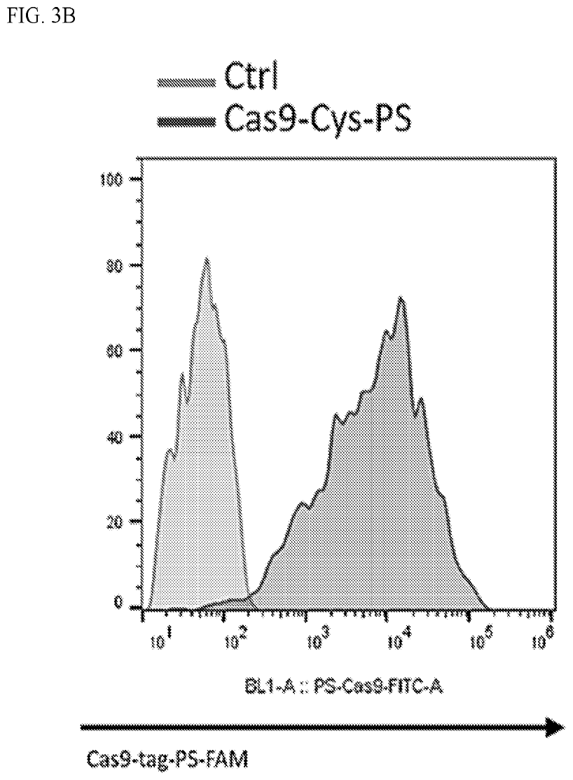

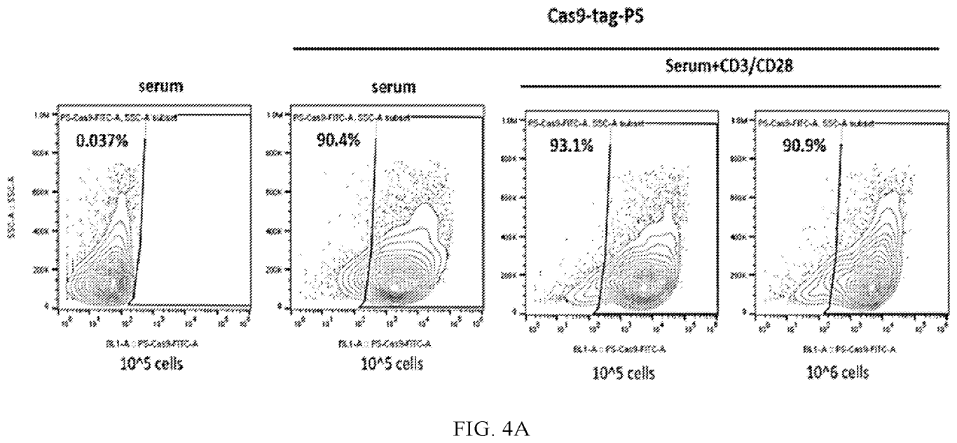

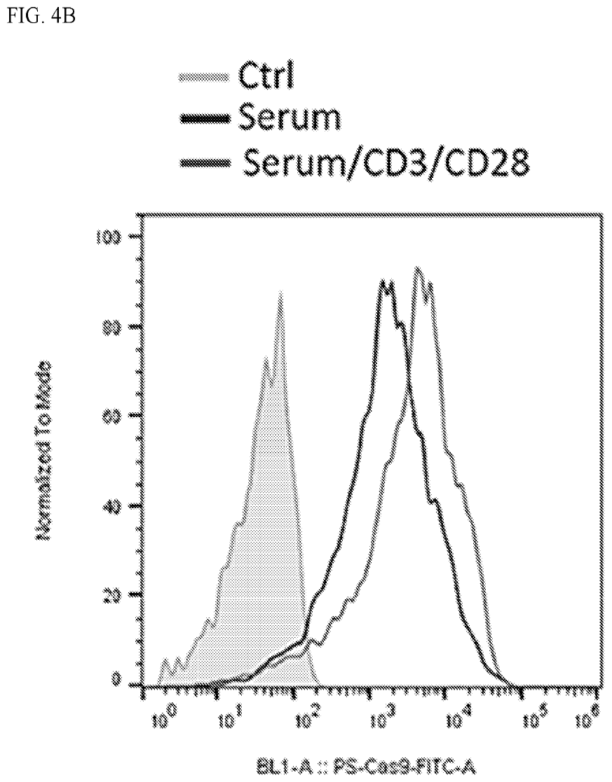

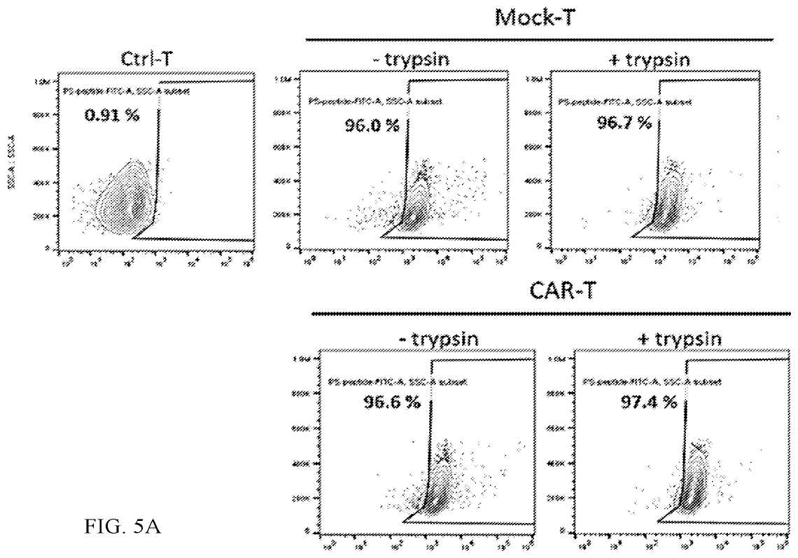

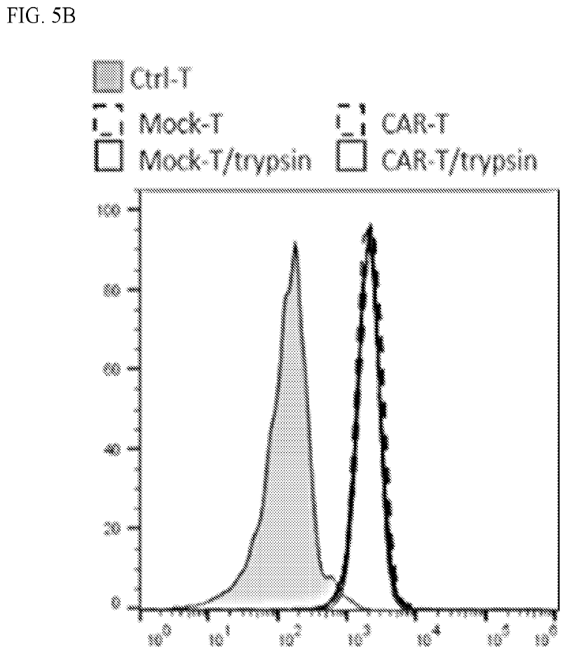

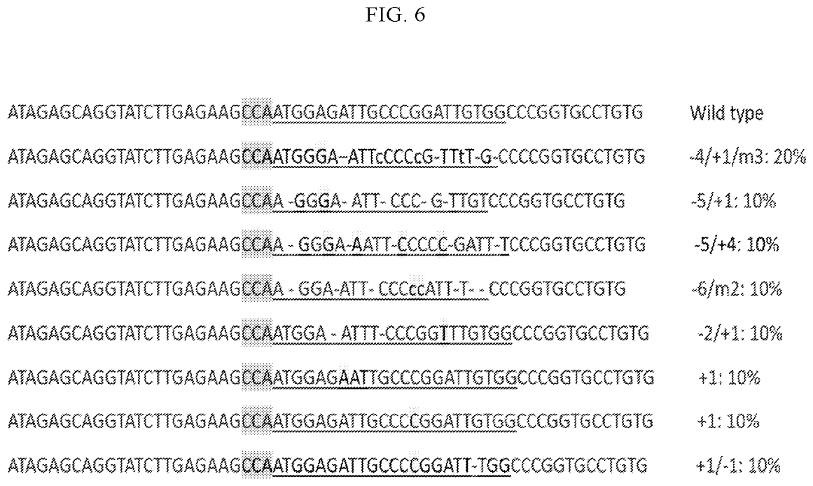

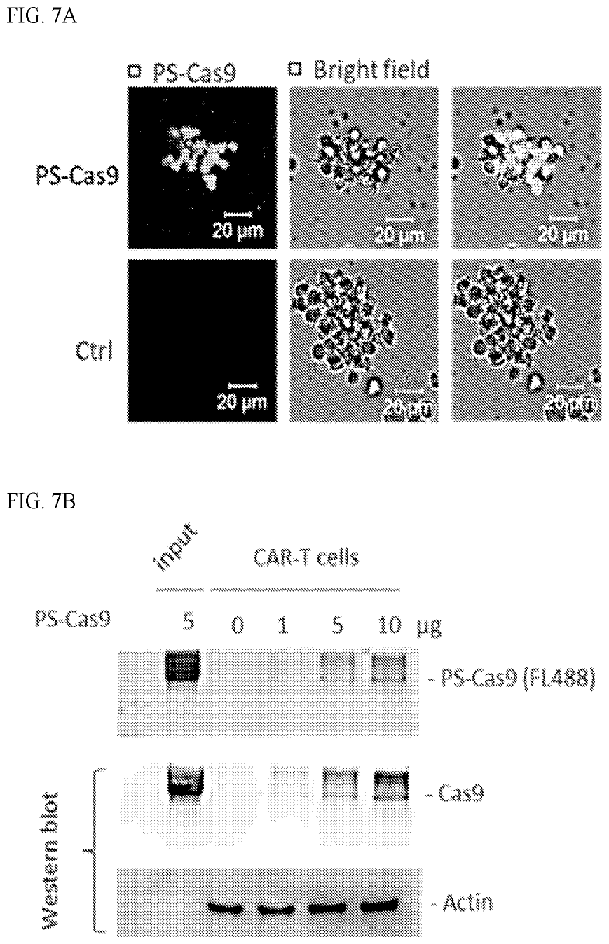

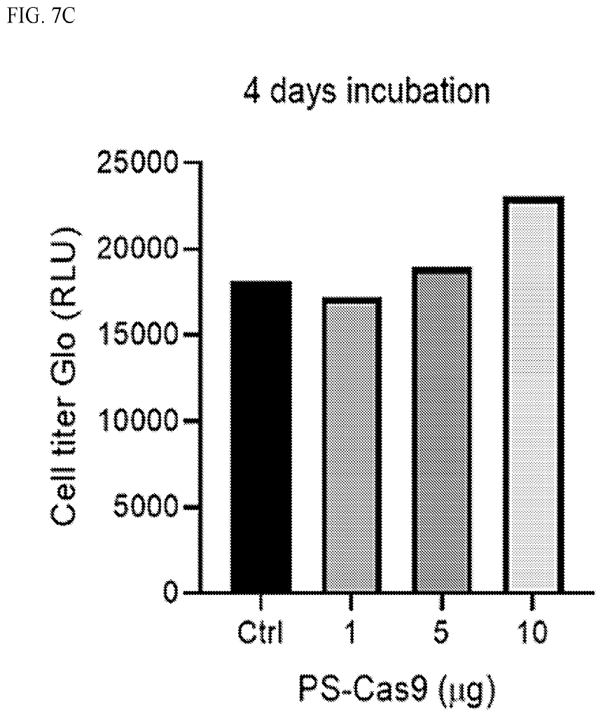

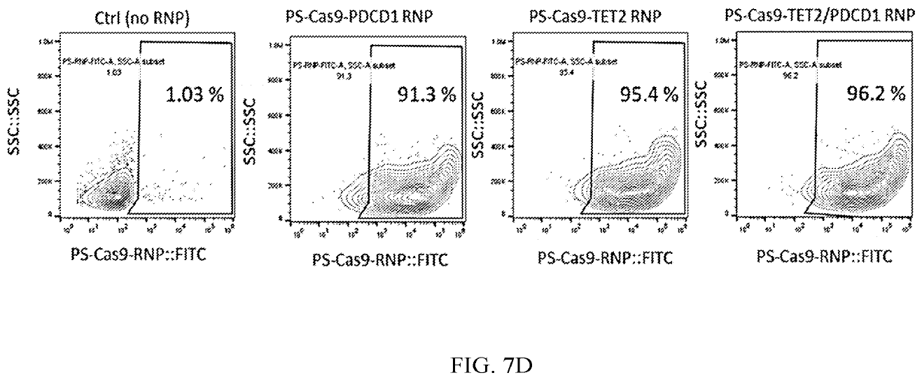

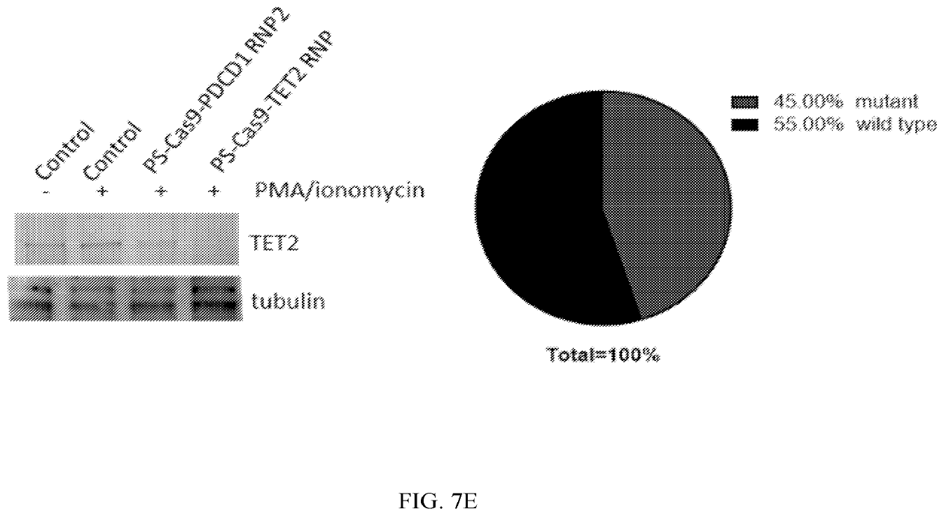

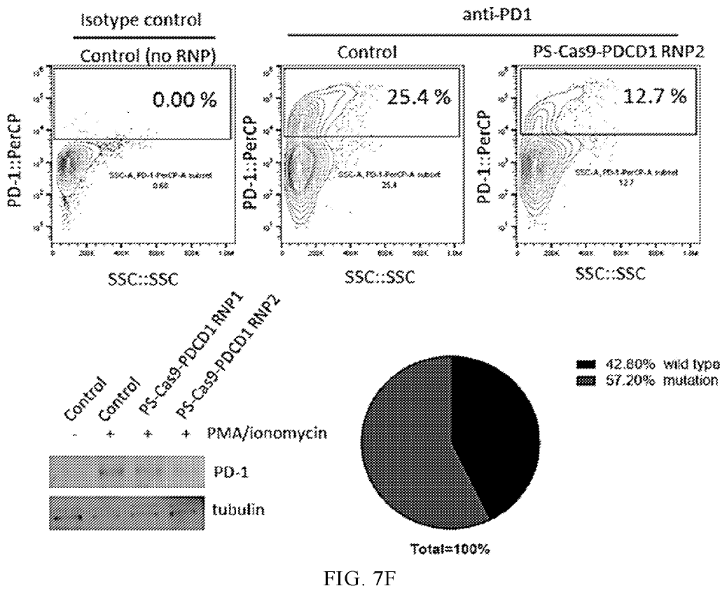

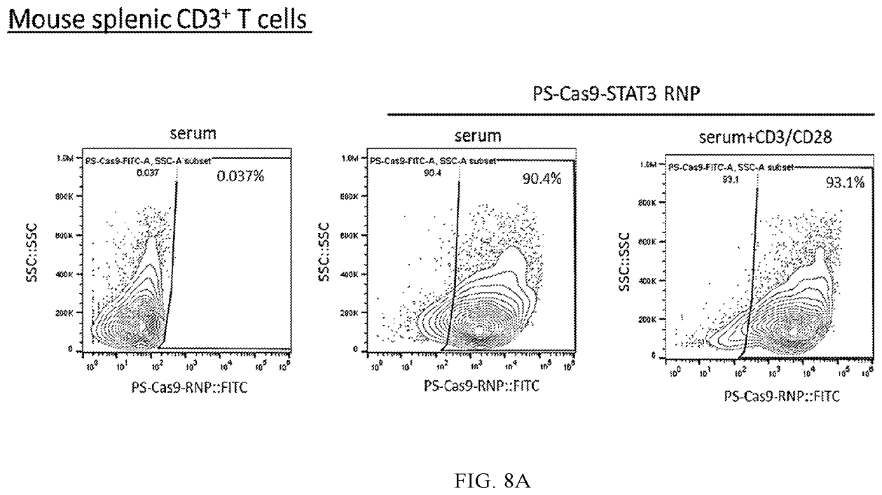

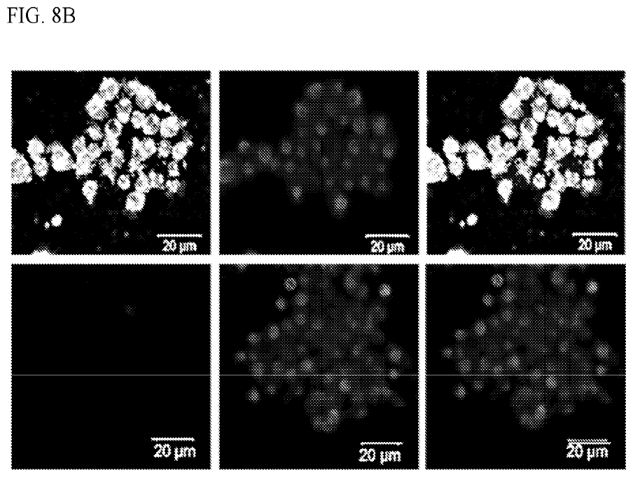

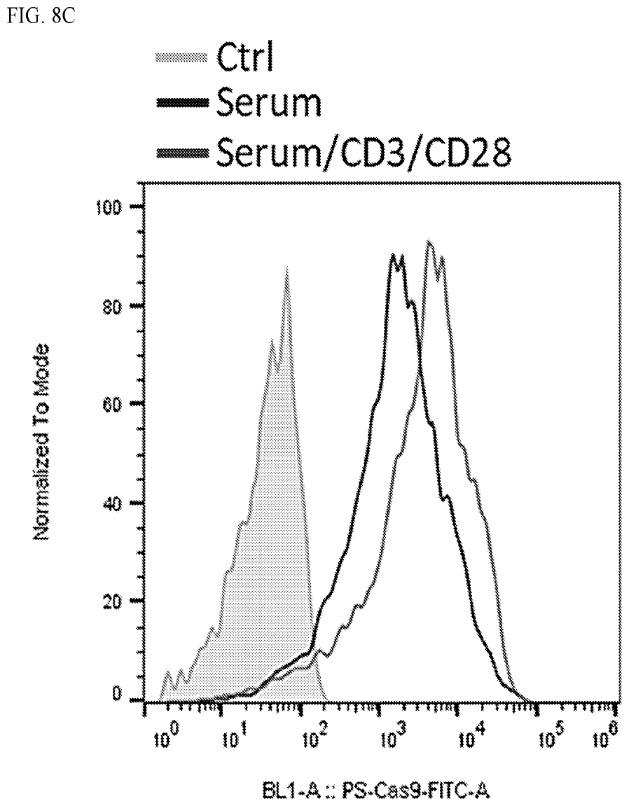

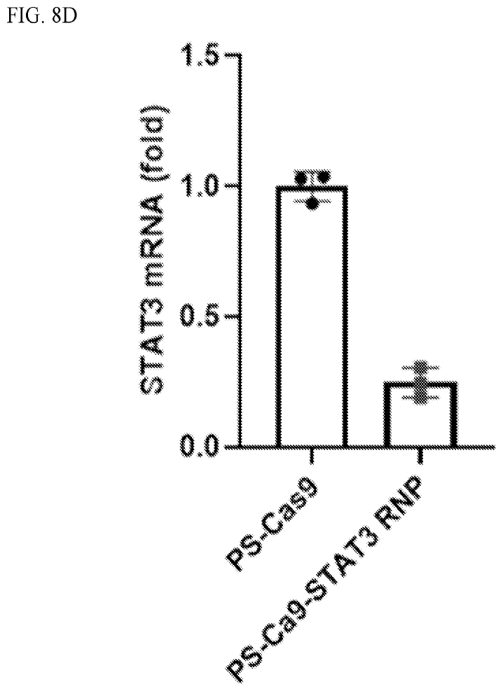

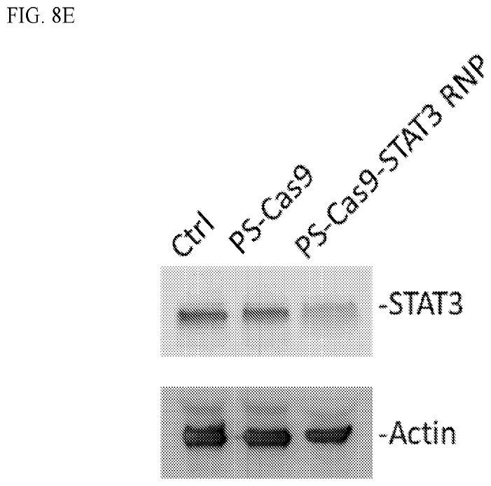

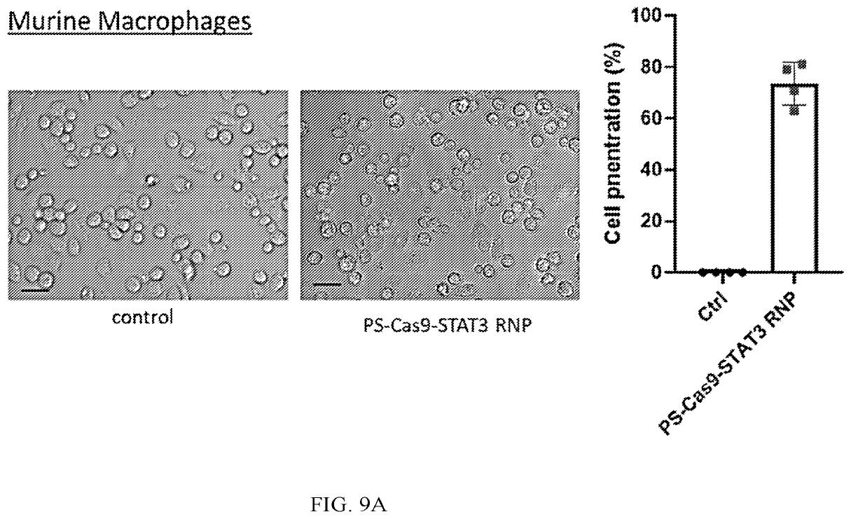

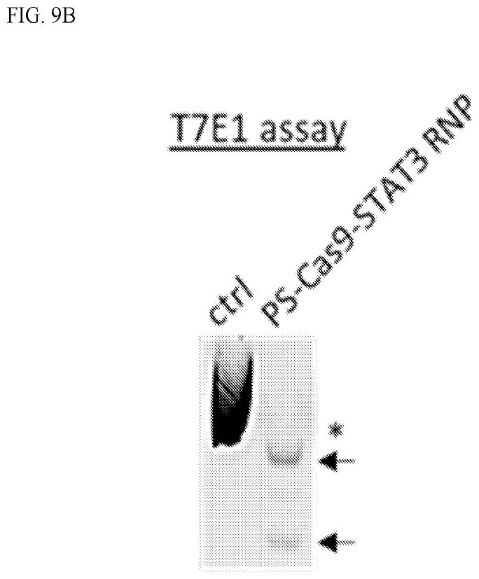

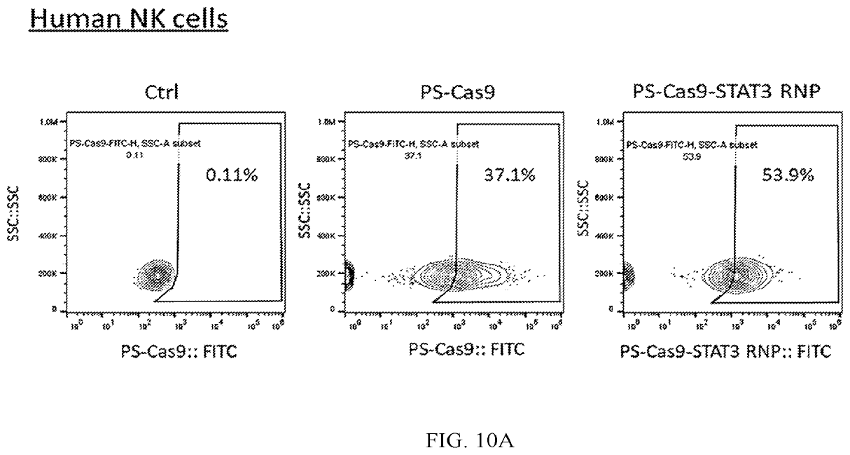

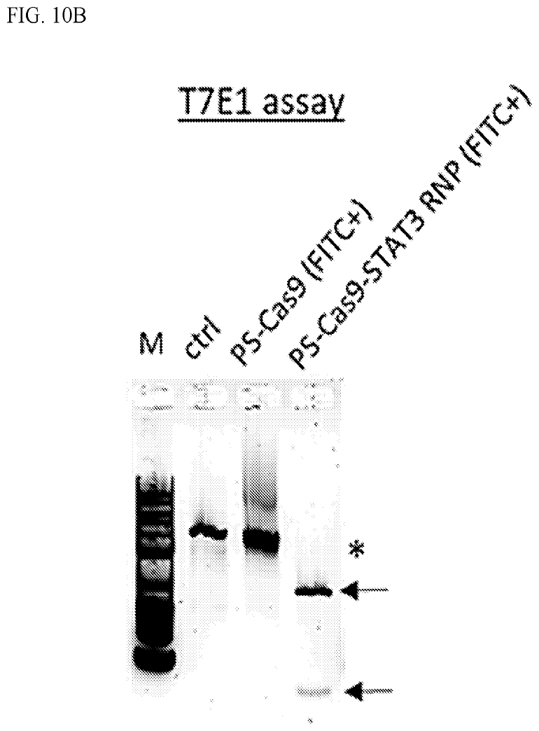

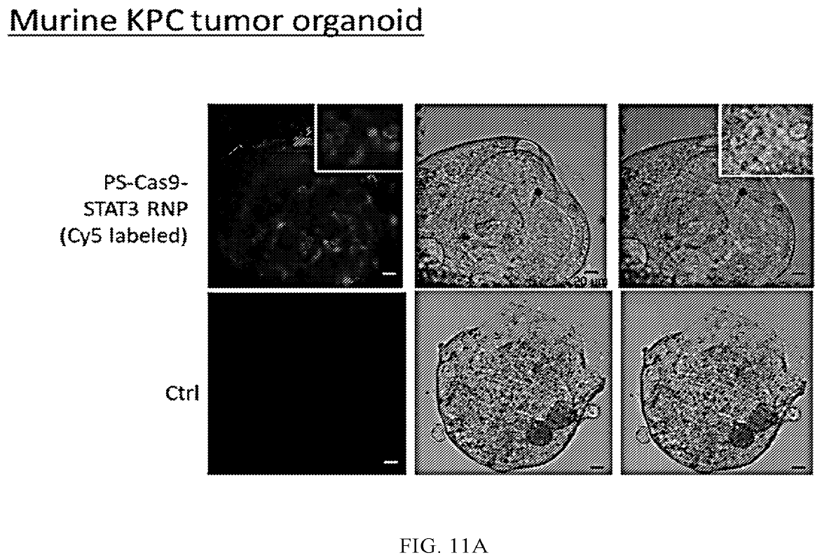

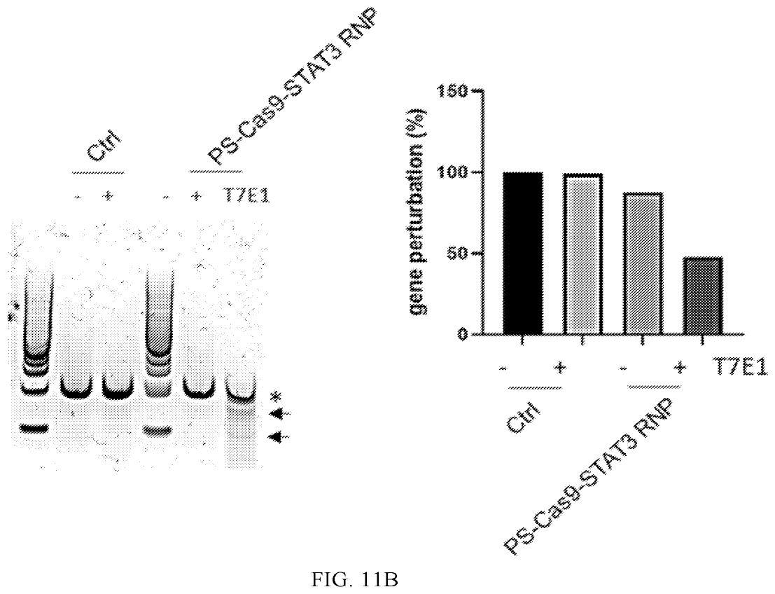

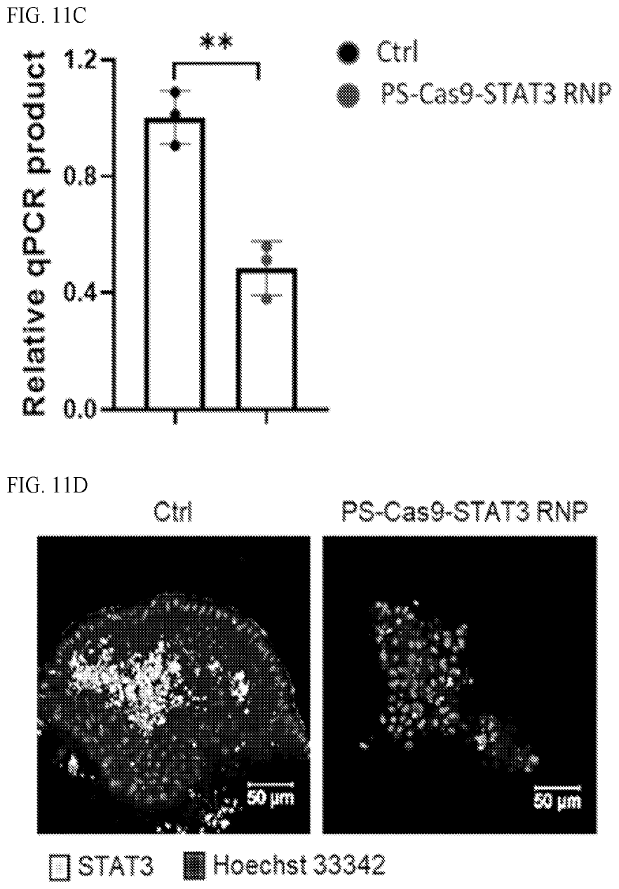

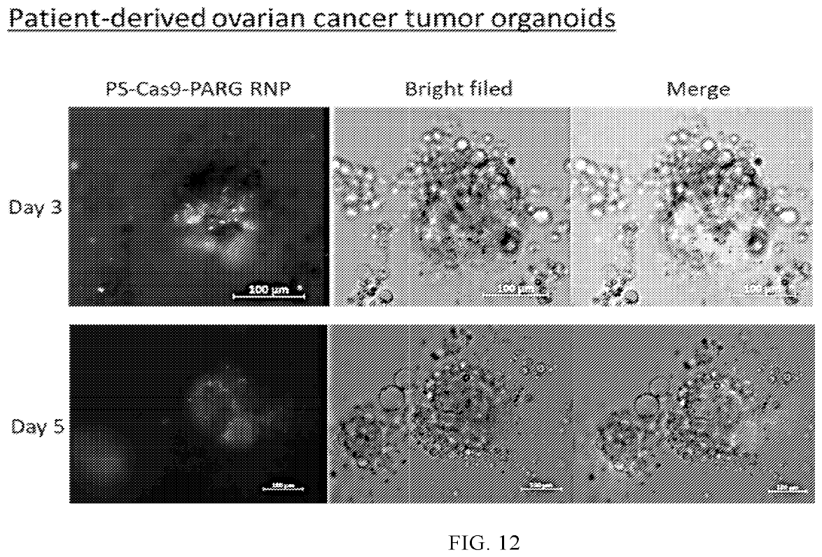

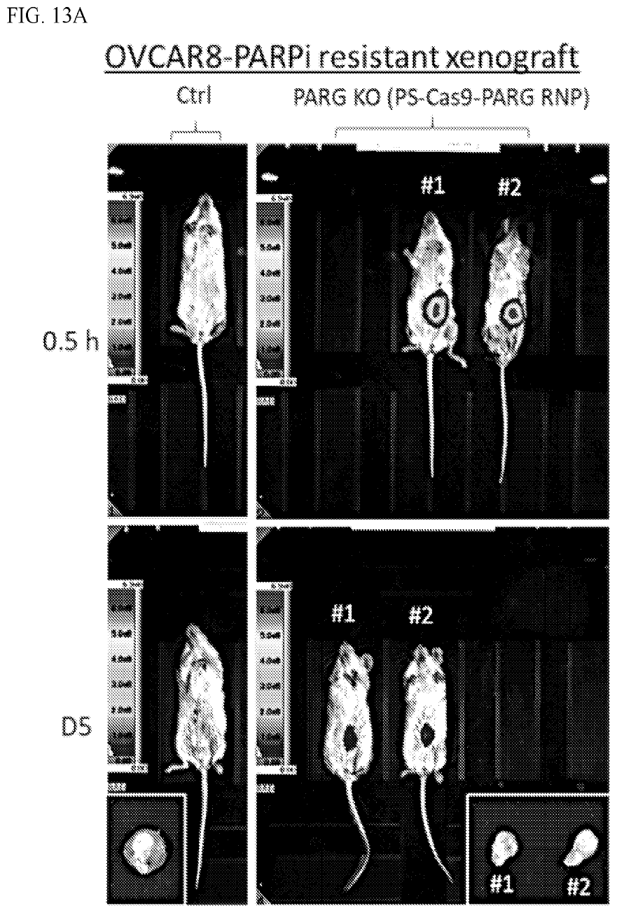

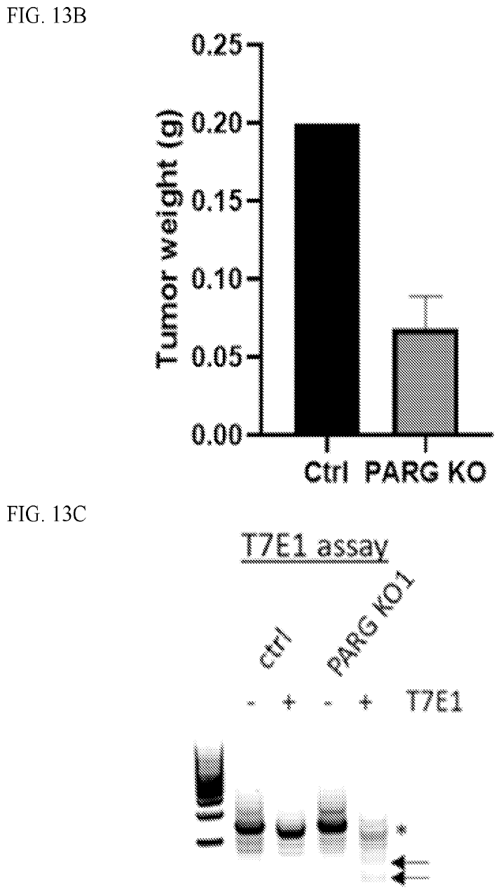

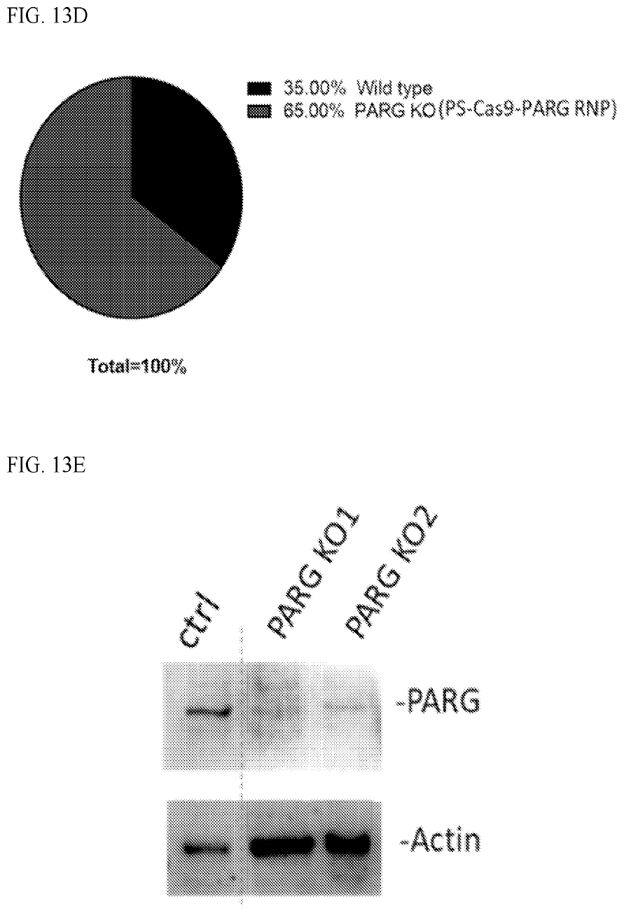

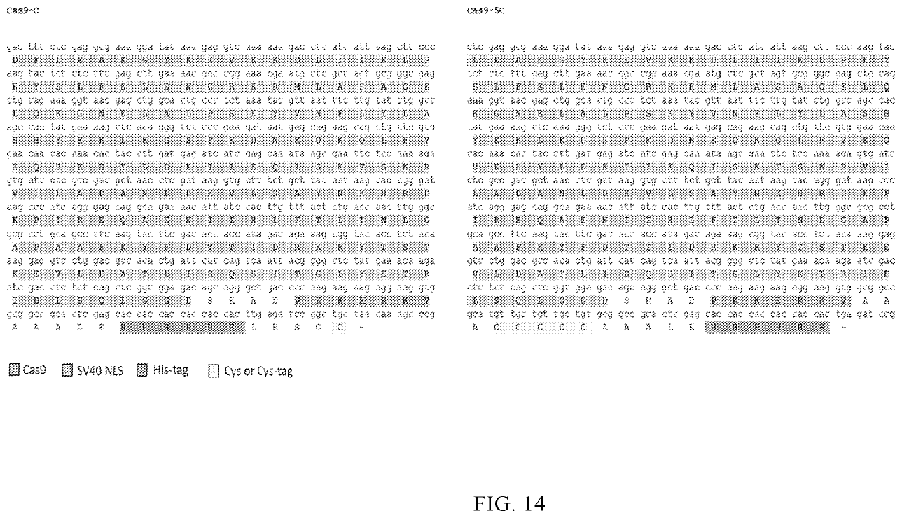

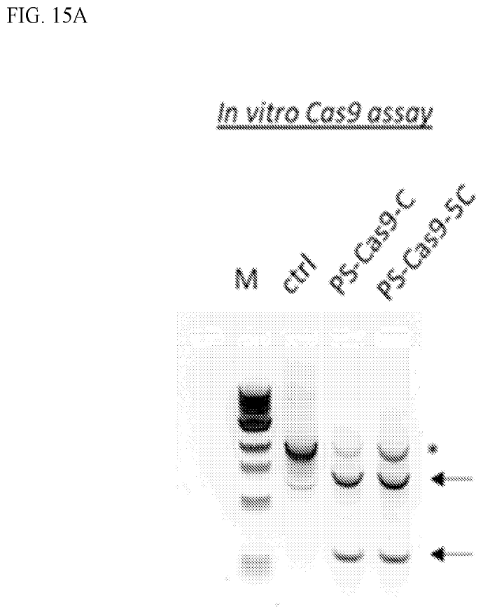

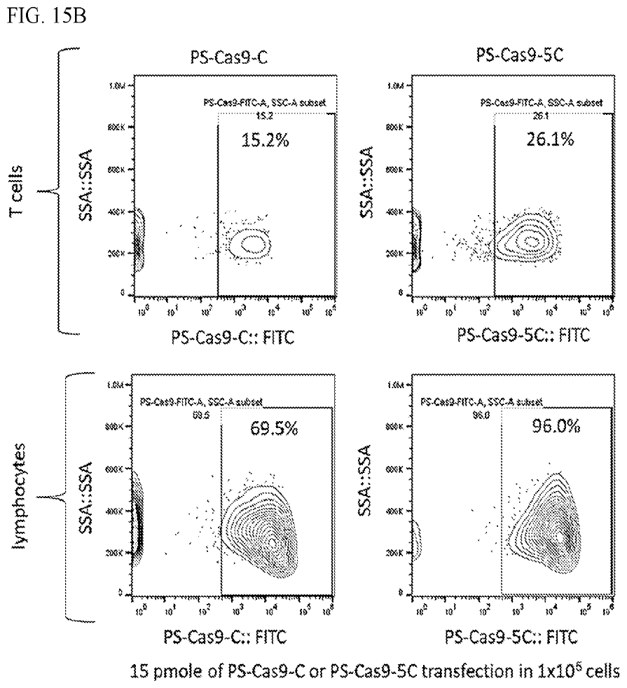

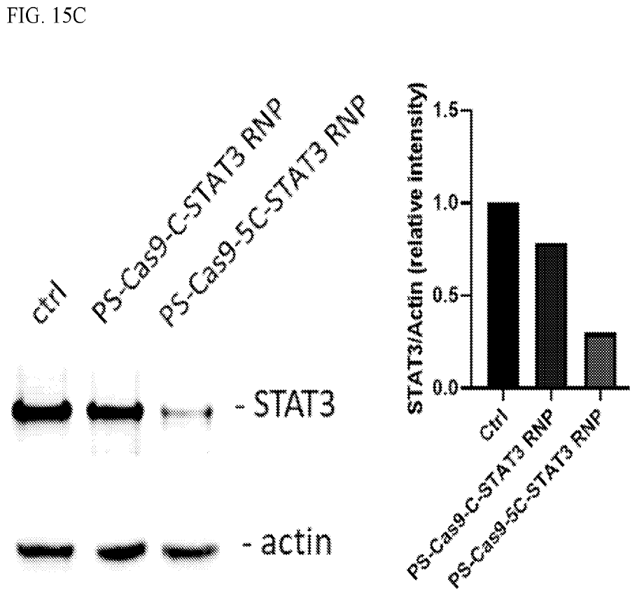

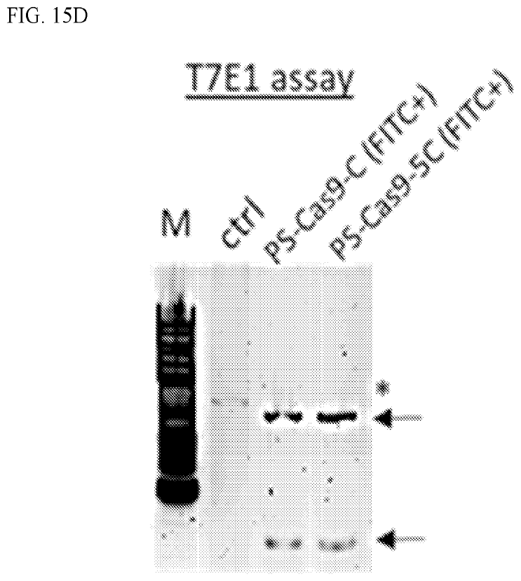

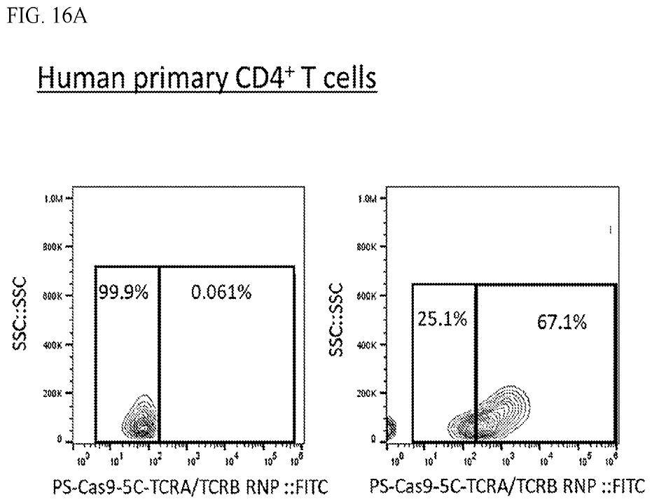

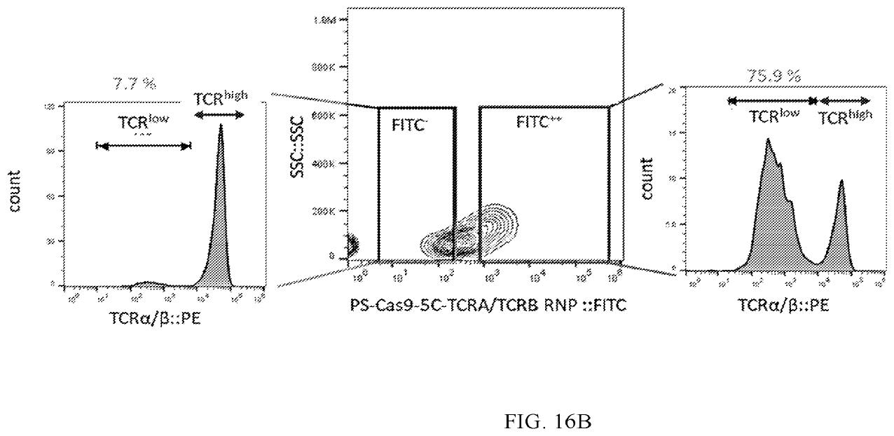

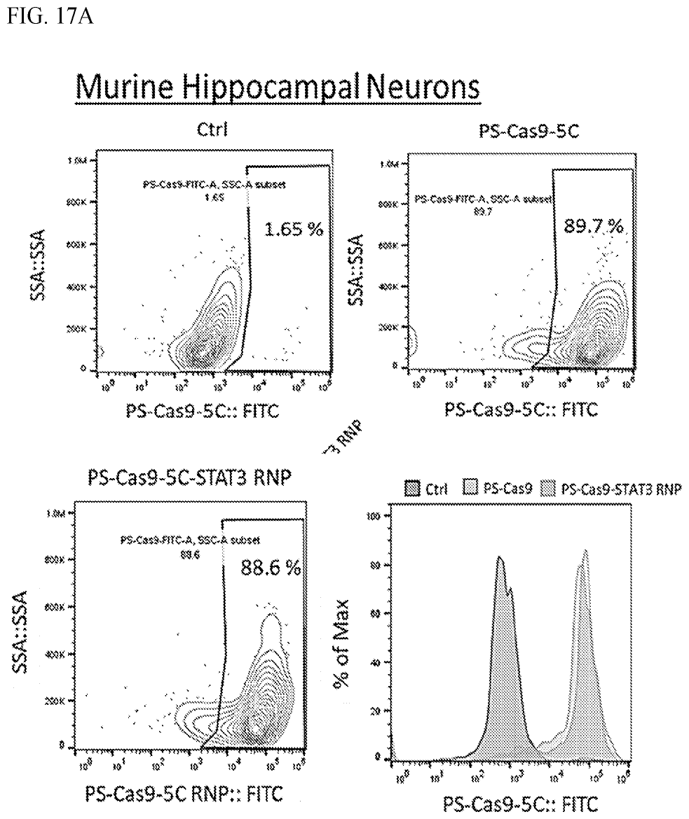

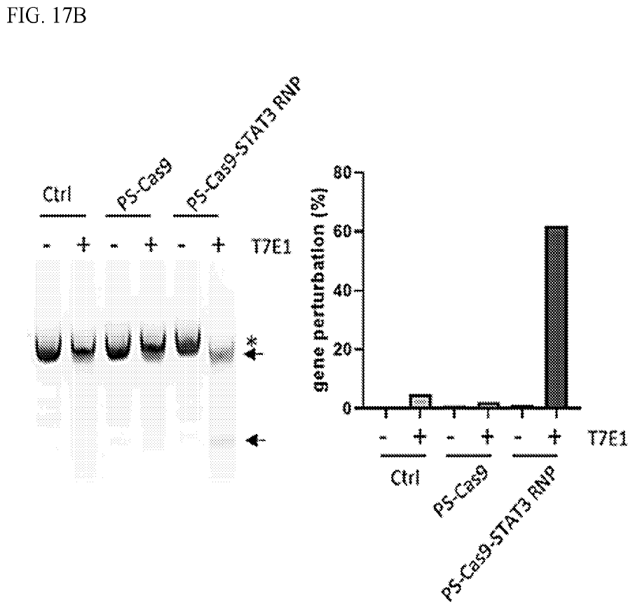

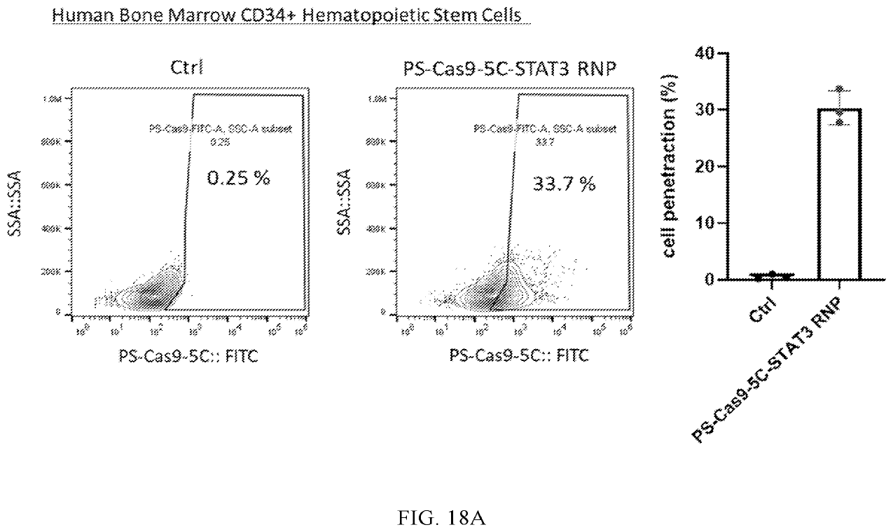

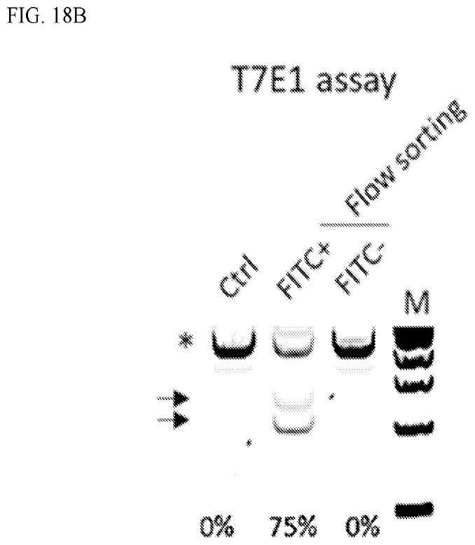

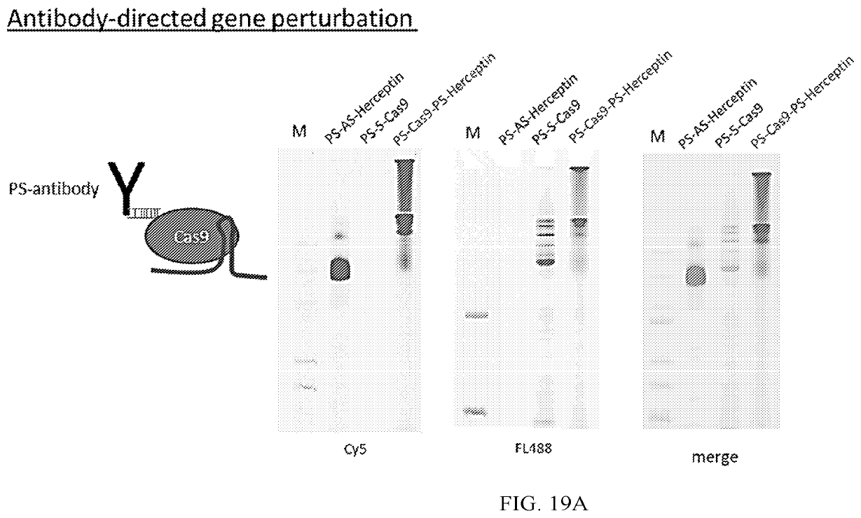

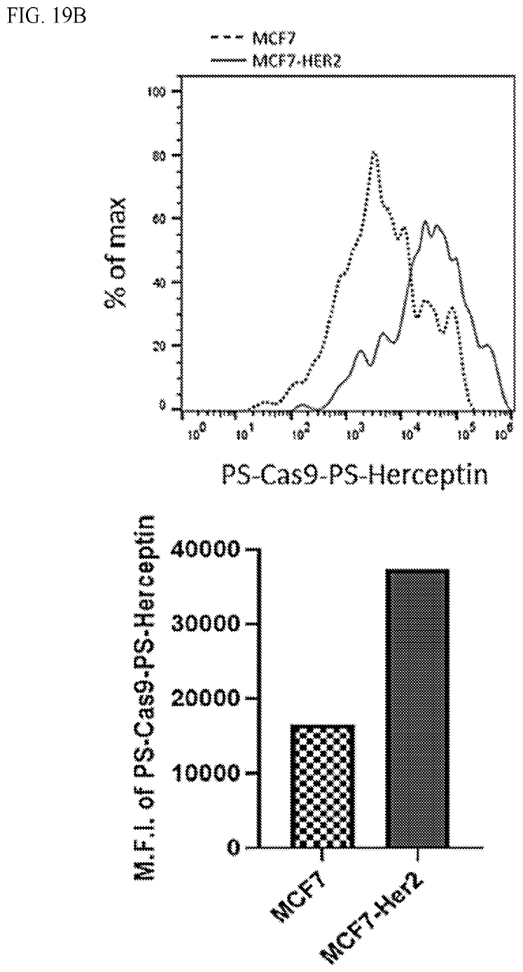

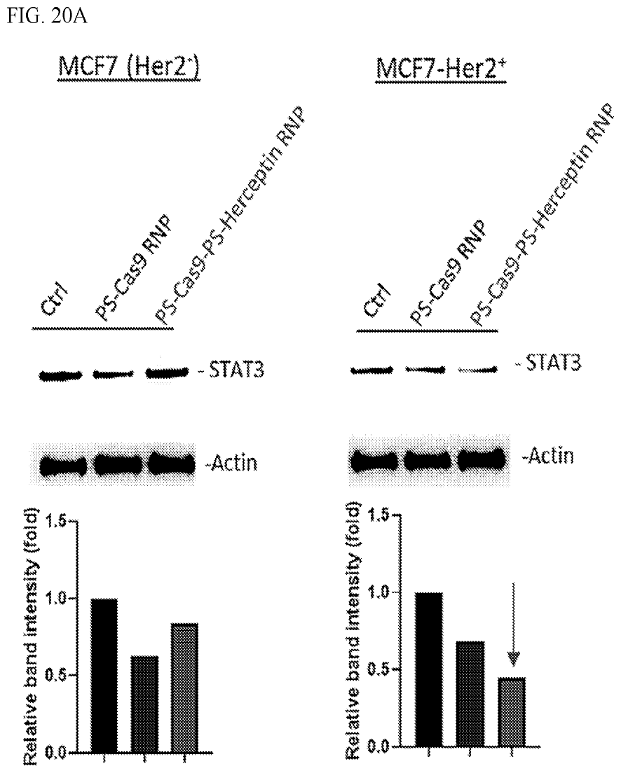

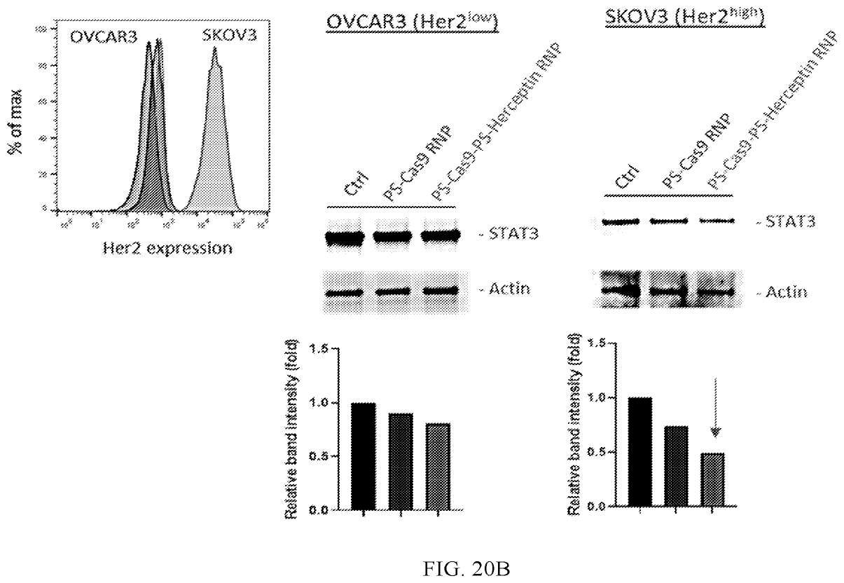

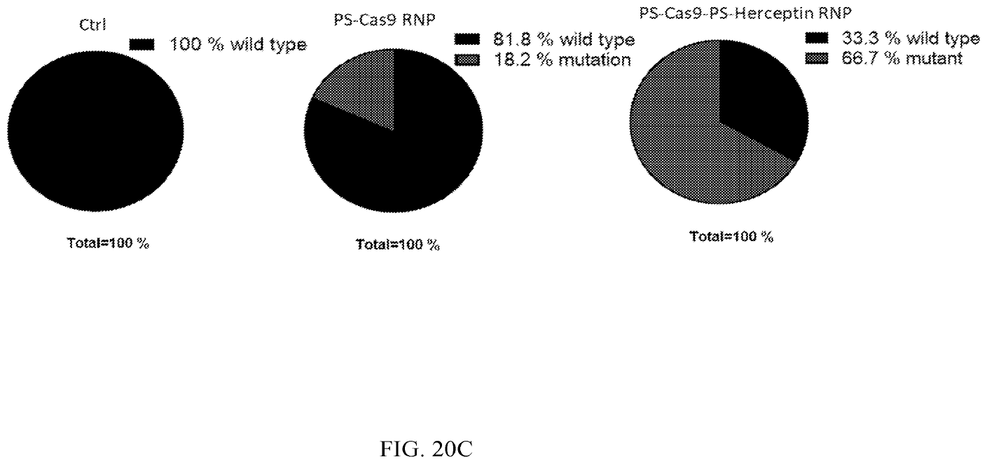

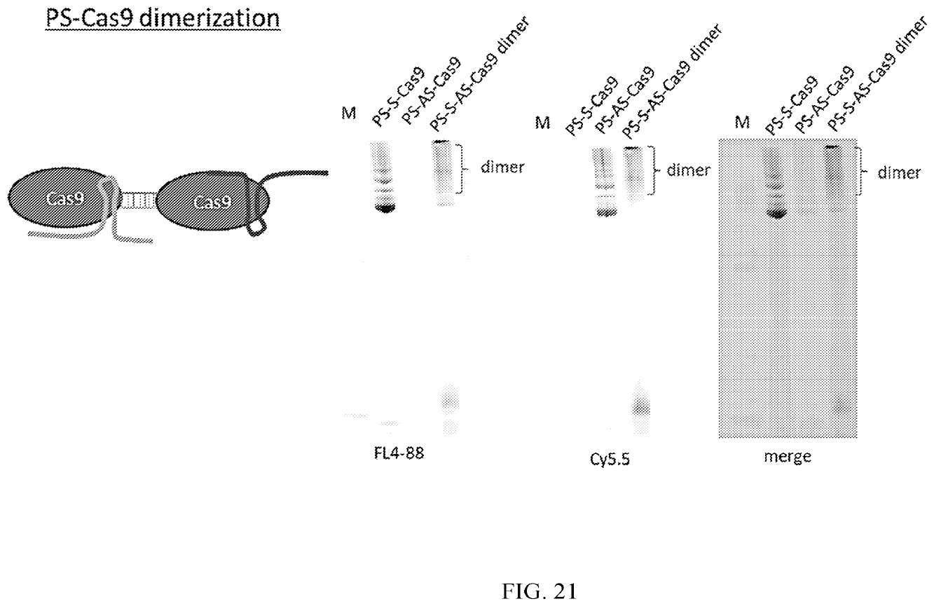

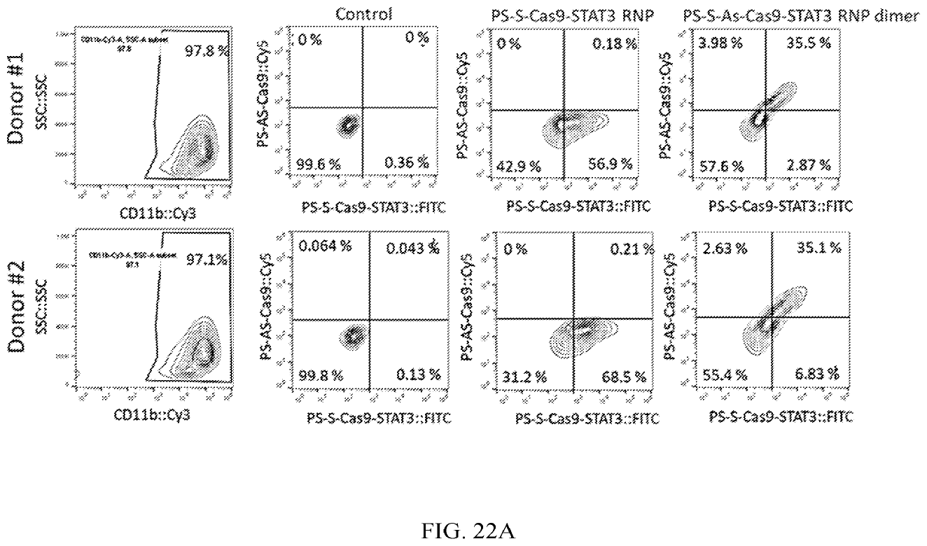

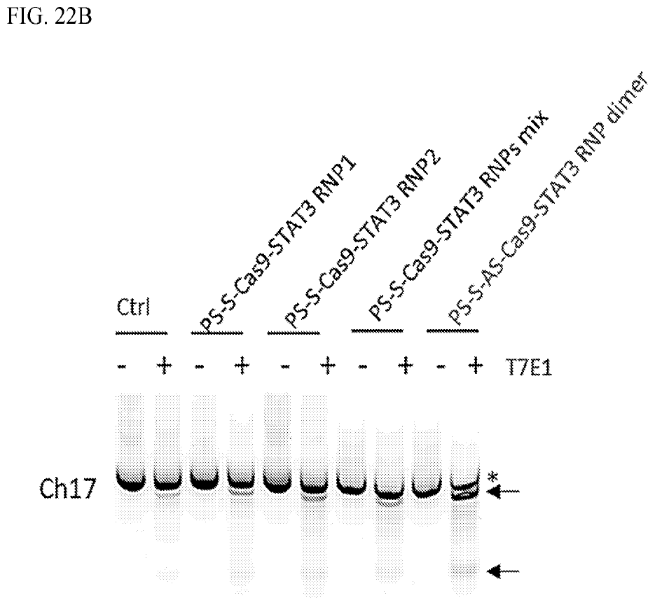

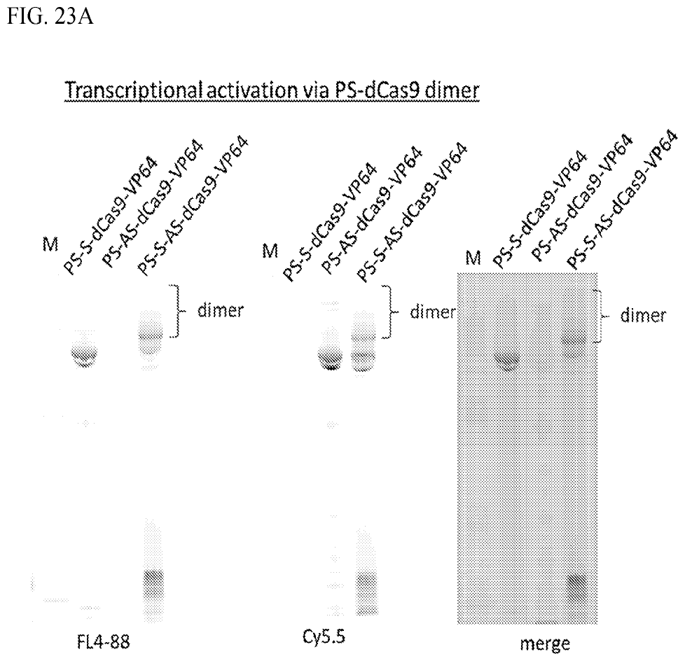

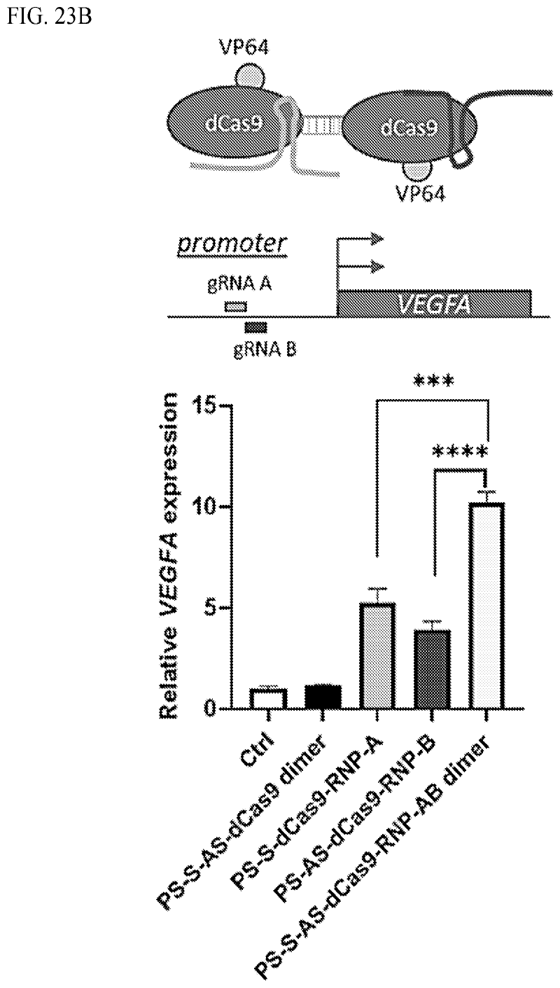

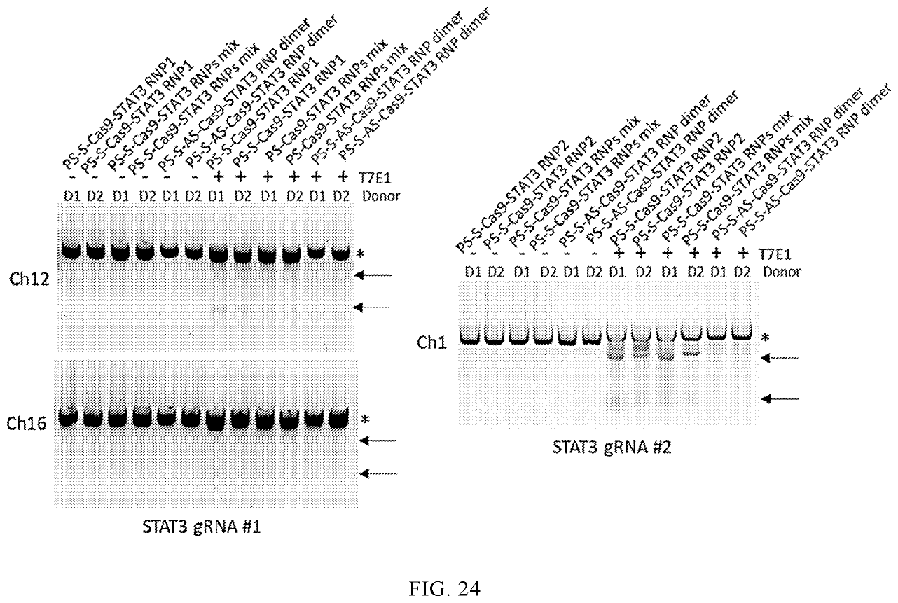

As illustrated in FIGS. 3A and 3B and FIGS. 4A and 4B, the Cas9-Cys-PS conjugate internalized into human malignant T cells at nearly 100% and further demonstrated great efficiency for internalizing into mouse splenic T cells. The cell penetrating Cas9-Cys-PS conjugate also penetrated CAR-T cells with high efficiency, and resulted in efficient targeted gene deletion/mutation by the modified Cas9/guide RNA ( 9/10=90%), as shown in FIGS. 5A and 5B. These results illustrate that delivering Cas9/STAT3 guide RNA to induce STAT3 functional knockout in T cells prior to bone marrow transplant may reduce graft vs host disease and increase antitumor immune responses.

### Example 2

In addition to making CAR-T cells more effective by deleting genes that are immunosuppressive and/or those can cause cytokine storms, the compositions and methods provided herein are used to make universal CAR-T cells. By deleting T cell receptors, MHCs, allogenic responses may be reduced. The feasibility of using this technology to generate universal CAR-T is shown in FIGS. 5A and 5B.

This approach is used to delete genes in T cells, hematopoietic stem cells, stem cells and other types of cells as shown in FIGS. 3A and 3B and FIGS. 4A and 4B.

The following attachment chemistries are alternative methods used to conjugate PS ssDNA-oligo to Cas9 through either covalent, disulfide or thioester bond formation. The chemistries include: Amine-to-amine, Amine-azide, Amine-aldehyde, Azide-aldehyde, Amine-to-sulfhydryl group, Sulfhydryl-tosulfhydryl group, Maleimide-to-sulfhydryl group.

Alternative methods to this approach include using multiple guide RNAs mixed with Cas9 enzymes to delete several genes. In addition to Cas9 enzyme, other CRISPR-associated protein endonucleases using similar chemistries to attach PS ssDNA-oligos are used for cell penetration and Cas9 gene deletion. This approach also applies to Transcription activator-like effector nucleases (TALEN) that can be engineered to cut specific sequences of DNA.

The compositions and methods provided herein are used to reduce off-target effects of Cas9 enzyme. Bi-specific Cas9 pair are generated: each Cas9 with a guide RNA flanking one end of the targeted region of a gene. One Cas9 enzyme is attached with a sense PS ssDNA-oligo and the other with an antisense PS ssDNA-oligo. The annealing of the PS ssDNA-oligos through complementarity enables the generation of a bi-specific Cas9/guide RNAs that binds to two independent regions of the targeted gene. If heating up during annealing is an issue for Cas9 enzyme activities, thermal-stable Cas proteins may be used.

In addition to gene deletion, the methods and compositions provided herein effectively internalize dead Cas9 enzyme through guide RNA to deliver activator and suppressor to regulate expression of immunostimulatory and immunosuppressive genes, respectively.

The cell-penetrating Cas9/guideRNA approach disclosed here may be used for in vivo treatments. For example, treating solid tumors through local injections to delete genes, such as STAT3, in tumor cells, tumor-associated immune cells and cancer associated fibroblasts, cancer stem cells and tumor endothelial cells, to block tumor cell growth and induce antitumor immune responses.

Exemplary embodiments of DNA editing agents forming parts of the complexes provided herein with potential attachment sites of the chemical linker are depicted:

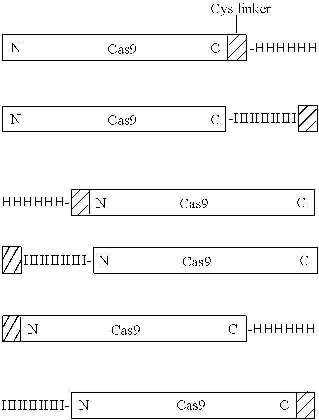

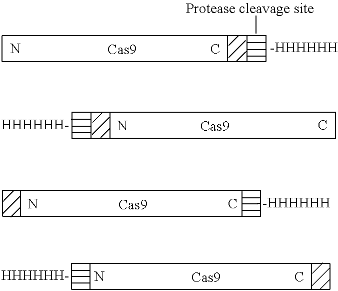

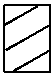

Cysteine linker; can be single or mulitple cysteine residues 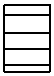
Protease cleavage site, including but not limited to TEV, thrombin and PreScission protease.

```
SEQ ID NO: 1 ACAATCCGGGCAATCTCCATTGG
SEQ ID NO: 2 (Cas9 from Streptococcus pyogenes (S. pyogenes))
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
SEQ ID NO: 3 (Cas9 from Streptococcus pyogenes (S. pyogenes)) with linker
between Cas9 and HIS tag in bold and underline
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGDSRADPKKKRKVAAACAAALEHHHHHH
SEQ ID NO: 4 (dCas9)
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD
SEQ ID NO: 5 (ddCasCfp1)
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQMPTLASGWDVNKEKNNGAILFVKNGLYYLGIMPKQKGRYKALSFEPTEK
TSEGFDKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFFFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLANLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRN
SEQ ID NO: 6 (ddLbCfp1)
MSKLEKFTNCYSLSKTLRFKAIPVGKTQENIDNKRLLVEDEKRAEDYKGVKKLLDR
YYLSFINDVLHSIKLKNLNNYISLFRKKTRTEKENKELENLEINLRKEIAKAFKGNEGY
KSLFKKDIIETILPEFLDDKDEIALVNSFNGFTTAFTGFFDNRENMFSEEAKSTSIAFRCI
NENLTRYISNMDIFEKVDAIFDKHEVQEIKEKILNSDYDVEDFFEGEFFNFVLTQEGID
VYNAIIGGFVTESGEKIKGLNEYINLYNQKTKQKLPKFKPLYKQVLSDRESLSFYGEG
YTSDEEVLEVFRNTLNKNSEIFSSIKKLEKLFKNFDEYSSAGIFVKNGPAISTISKDIFG
EWNVIRDKWNAEYDDIHLKKKAVVTEKYEDDRRKSFKKIGSFSLEQLQEYADADLS
VVEKLKEIIIQKVDEIYKVYGSSEKLFDADFVLEKSLKKNDAVVAIMKDLLDSVKSFE
NYIKAFFGEGKETNRDESFYGDFVLAYDILLKVDHIYDAIRNYVTQKPYSKDKFKLY
FQNPQFMGGWDKDKETDYRATILRYGSKYYLAIMDKKYAKCLQKIDKDDVNGNYE
KINYKLLPGPNKMLPKVFFSKKWMAYYNPSEDIQKIYKNGTFKKGDMFNLNDCHKL
IDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRA
SLKKEELVVHPANSPIANKNPDNPKKTTTLSYDVYKDKRFSEDQYELHIPIAINKCPK
NIFKINTEVRVLLKHDDNPYVIGIARGERNLLYIVVVDGKGNIVEQYSLNEIINNENGI
RIKTDYHSLLDKKEKERFEARQNWTSIENIKELKAGYISQVVHKICELVEKYDAVIAL
ADLNSGFKNSRVKVEKQVYQKFEKMLIDKLNYMVDKKSNPCATGGALKGYQITNK
FESFKSMSTQNGFIFYIPAWLTSKIDPSTGFVNLLKTKYTSIADSKKFISSFDRIMYVPE
EDLFEFALDYKNFSRTDADYIKKWKLYSYGNRIRIFRNPKKNNVFDWEEVCLTSAYK
ELFNKYGINYQQGDIRALLCEQSDKAFYSSFMALMSLMLQMRNSITGRTDVDFLISP
VKNSDGIFYDSRNYEAQENAILPKNADANGAYNIARKVLWAIGQFKKAEDEKLDKV
KIAISNKEWLEYAQTSVKH
SEQ ID NO: 7 (ddFnCfp1)
MYPYDVPDYASGSGMSIYQEFVNKYSLSKTLRFELIPQGKTLENIKARGLILDDEKRA
KDYKKAKQIIDKYHQFFIEEILSSVCISEDLLQNYSDVYFKLKKSDDDNLQKDFKSAK
DTIKKQISEYIKDSEKFKNLFNQNLIDAKKGQESDLILWLKQSKDNGIELFKANSDITD
IDEALEIIKSFKGWTTYFKGFHENRKNVYSSNDIPTSIIYRIVDDNLPKFLENKAKYESL
KDKAPEAINYEQIKKDLAEELTFDIDYKTSEVNQRVFSLDEVFEIANFNNYLNQSGIT
KFNTIIGGKFVNGENTKRKGINEYINLYSQQINDKTLKKYKMSVLFKQILSDTESKSF
VIDKLEDDSDVVTTMQSFYEQIAAFKTVEEKSIKETLSLLFDDLKAQKLDLSKIYFKN
DKSLTDLSQQVFDDYSVIGTAVLEYITQQIAPKNLDNPSKKEQELIAKKTEKAKYLSL
ETIKLALEEFNKHRDIDKQCRFEEILANFAAIPMIFDEIAQNKDNLAQISIKYQNQGKK
DLLQASAEDDVKAIKDLLDQTNNLLHKLKIFHISQSEDKANILDKDEHFYLVFEECYF
ELANIVPLYNKIRNYITQKPYSDEKFKLNFENSTLANGWDKNKEPDNTAILFIKDDKY
YLGVMNKKNNKIFDDKAIKENKGEGYKKIVYKLLPGANKMLPKVFFSAKSIKFYNP
SEDILRIRNHSTHTKNGSPQKGYEKFEFNIEDCRKFIDFYKQSISKHPEWKDFGFRFSD
TQRYNSIDEFYREVENQGYKLTFENISESYIDSVVNQGKLYLFQIYNKDFSAYSKGRP
NLHTLYWKALFDERNLQDVVYKLNGEAELFYRKQSIPKKITHPAKEAIANKNKDNP
KKESVFEYDLIKDKRFTEDKFFFHCPITINFKSSGANKFNDEINLLLKEKANDVHILSIA
RGERHLAYYTLVDGKGNIIKQDTFNIIGNDRMKTNYHDKLAAIEKDRDSARKDWKK
INNIKEMKEGYLSQVVHEIAKLVIEYNAIVVFEDLNFGFKRGRFKVEKQVYQKLEKM
LIEKLNYLVFKDNEFDKTGGVLRAYQLTAPFETFKKMGKQTGIIYYVPAGFTSKICPV
TGFVNQLYPKYESVSKSQEFFSKFDKICYNLDKGYFEFSFDYKNFGDKAAKGKWTIA
SFGSRLINFRNSDKNHNWDTREVYPTKELEKLLKDYSIEYGHGECIKAAICGESDKKF
FAKLTSVLNTILQMRNSKTGTELDYLISPVADVNGNFFDSRQAPKNMPQDADANGA
YHIGLKGLMLLGRIKNNQEGKKLNLVIKNEEYFEFVQNRNN
SEQ ID NO: 8 Wild type:
ATAGAGCAGGTATCTTGAGAAGCCAATGGAGATTGCCCGGATTGTGGCCCGGTG
CCTGTG
SEQ ID NO: 9 Mutation 1:
ATAGAGCAGGTATCTTGAGAAGCCAATGGGAATTcCCCcGTTtTGCCGGTGCCTGT
G
SEQ ID NO: 10 Mutation 2:
ATAGAGCAGGTATCTTGAGAAGCCAAGGGAATTCCCGTTGTGGCCCGGTGCCTG
TG
SEQ ID NO: 11 Mutation 3:
ATAGAGCAGGTATCTTGAGAAGCCAAGGGAAATTCCCCCGATTTGGCCCGGTGC
CTGTG
SEQ ID NO: 12 Mutation 4:
ATAGAGCAGGTATCTTGAGAAGCCAAGGAATTCCCccATTTCCCGGTGCCTGTG
SEQ ID NO: 13 Mutation 5:
ATAGAGCAGGTATCTTGAGAAGCCAATGGAATTCCCGGATTTGTGGCCCGGTGC
CTGTG
SEQ ID NO: 14 Mutation 6:
ATAGAGCAGGTATCTTGAGAAGCCAATGGAGAATTGCCCGGATTGTGGCCCGGT
GCCTGTG
SEQ ID NO: 15 Mutation 7:
ATAGAGCAGGTATCTTGAGAAGCCAATGGAGATTGCCCCGGATTGTGGCCCGGT
GCCTGTG
SEQ ID NO: 16 Mutation 8:
ATAGAGCAGGTATCTTGAGAAGCCAATGGAGATTGCCCCGGATTTGGCCCGGTG
CCTGTG
SEQ ID NO: 17 Cas9-C construct nucleic acid (nucleic acid of the Cas9-C alignment
shown)
SEQ ID NO: 18 Cas9-C construct protein sequence (amino acid sequence of the
Cas9-C alignment shown)
Cas9-C
gac ttt ctc gag gcg aaa gga tat aaa gag gtc aaa aaa gac ctc atc att aag ctt ccc
  D F L E A K G Y K E V K K D L I I K L P
aag tac tct ctc ttt gag ctt gaa aac ggc cgg aaa cga atg ctc gct agt gcg ggc gag
  K Y S L F E L E N G R K R M L A S A G E
ctg cag aaa ggt aac gag ctg gca ctg ccc tot aaa tac gtt aat ttc ttg tat ctg gcc
  L Q K G N E L A L P S K Y V N F L Y L A
agc cac tat gaa aag ctc aaa ggg tot ccc gaa gat aat gag cag aag cag ctg ttc gtg
  S H Y E K L K G S P E D N E Q K Q L F V
gaa caa cac aaa cac tac ctt gat gag atc atc gag caa ata agc gaa ttc tcc aaa aga
  E Q H K H Y L D E I I E Q I S E F S K R
gtg atc ctc gcc gac gct aac ctc gat aag gtg ctt tct gct tac aat aag cac agg gat
  V I L A D A N L D K V L S A Y N K H R D
aag ccc atc agg gag cag gca gaa aac att atc cac ttg ttt act ctg acc aac ttg ggc
  K P I R E Q A E N I I H L F T L T N L G
gcg cct gca gcc ttc aag tac ttc gac acc acc ata gac aga aag cgg tac acc tct aca
  A P A A F K Y F D T T I D R K R Y T S T
aag gag gtc ctg gac gcc aca ctg att cat cag tca att acg ggg cto tat gaa aca aga
  K E V L D A T L I H Q S I T G L Y E T R
atc gac ctc tct cag ctc ggt gga gac agc agg gct gac ccc aag aag aag agg aag gtg
  I D L S Q L G G D S R A D P K K K R K V
gcg gcc gca ctc gag cac cac cac cac cac cac ttg aga tcc ggc tgc taa caa agc ccg
  A A A L E H H H H H H L R S G C -
SEQ ID NO: 19: SV40 NLS P K K K R K V
SEQ ID NO: 20: His Tag H H H H H H
SEQ ID NO: 21 Cas9-5C construct nucleic acid (nucleic acid of the Cas9-5C
alignment shown)
SEQ ID NO: 22 Cas9-5C construct protein sequence (amino acid sequence of the
Cas9-5C alignment shown)
Cas9-5C
ctc gag gcg aaa gga tat aaa gag gtc aaa aaa gac ctc atc att aag ctt ccc aag tac
  L E A K G Y K E V K K D L I I K L P K Y
tct ctc ttt gag ctt gaa aac ggc cgg aaa cga atg ctc gct agt gcg ggc gag ctg cag
  S L F E L E N G R K R M L A S A G E L Q
aaa ggt aac gag ctg gca ctg ccc tct aaa tac gtt aat ttc ttg tat ctg gcc age cac
  K G N E L A L P S K Y V N F L Y L A S H
tat gaa aag ctc aaa ggg tct ccc gaa gat aat gag cag aag cag ctg ttc gtg gaa caa
  Y E K L K G S P E D N K Q K Q L F V E Q
cac aaa cac tac ctt gat gag atc atc gag caa ata agc gaa ttc tcc aaa aga gtg atc
  H K H Y L D E I I E Q I S E F S K R V I
ctc gcc gac gct aac ctc gat aag gtg ctt tct gct tac aat aag cac agg gat aag ccc
  L A D A N L D K V L S A Y N K H R D K P
atc agg gag cag gca gaa aac att atc cac ttg ttt act ctg acc aac ttg ggc gcg cct
  I R E Q A E N I I H L F T L T N L G A P
gca gcc ttc aag tac ttc gac acc acc ata gac aga aag cgg tac acc tct aca aag gag
  A A F K Y F D T T I D R K R Y T S T K E
gtc ctg gac gcc aca ctg att cat cag tca att acg ggg ctc tat gaa aca aga atc gac
  V L D A T L I H Q S I T G L Y E T R I D
ctc tct cag ctc ggt gga gac agc agg gct gac ccc aag aag aag agg aag gtg gcg gcc
  L S Q L G G D S R A D P K K K R K V A A
gca tgt tgc tgt tgc tgt gcg gcc gca ctc gag cac cac cac cac cac cac tga gat ccg
  A C C C C C A A A L E H H H H H H -
SEQ ID NO: 23 Cys Tag C C C C C
SEQ ID NO: 24 Cas9-C nucleic acid (nucleic acid sequence shown in bold italic in
Cas9-C alignment)
SEQ ID NO: 25 Cas9-C protein acid (amino acid sequence shown in bold italic in
Cas9-C alignment)
SEQ ID NO: 26 Cas9-5C nucleic acid (nucleic acid sequence shown in bold italic in
Cas9-5C alignment)
SEQ ID NO: 27 Cas9-5C protein acid (amino acid sequence shown in bold italic in
Cas9-5C alignment)
```

| Description | Sequence | SEQ ID NO: |
|---|---|---|
| Sense phosphorothioate | T*C*C*A*T*G*A*G*C*T*T*C*C*T*G*A*T*G*C*T | SEQ ID NO: 28 |
| oligonucleotide | | |
| Anti-sense phosphorothioate | A*G*C*A*T*C*A*G*G*A*A*G*C*T*C*A*T*G*G*A | SEQ ID NO: 29 |
| oligonucleotide | | |
| gRNA for PS-Cas9-STAT3 RNP | ACAATCCGGGCAATCTCCATTGG | SEQ ID NO: 30 |
| gRNA for PS-Cas9-PDCD1 RNP | CATGTGGAAGTCACGCCCGTTGG | SEQ ID NO: 31 |
| gRNA for PS-Cas9-PDCD2 RNP | CCCCTTCGGTCACCACGAGCAGG | SEQ ID NO: 32 |
| gRNA for PS-Cas9-TET2 RNP | GGATAGAACCAACCATGTTGAGG | SEQ ID NO: 33 |
| gRNA for PS-Cas9-PARG RNP | ACCAGTTGGATGGACACTAAAGG | SEQ ID NO: 34 |
| gRNA for PS-Cas9-5C- | AGAGTCTCTCAGCTGGTACACGG | SEQ ID NO: 35 |
| TCRA/TCRB RNP (TCRA) | | |
| gRNA for PS-Cas9-5C- | GCAGTATCTGGAGTCATTGAGGG | SEQ ID NO: 36 |
| TCRA/TCRB RNP (TCRB) | | |
| gRNA for PS-S-Cas9-STAT3 | CATTCGACTCTTGCAGGAAGCGG | SEQ ID NO: 37 |
| RNP1 | | |
| gRNA for PS-S-Cas9-STAT3 | AGTGGCATGTGATTCTTTGCTGG | SEQ ID NO: 38 |
| RNP2 | | |
| gRNA for PS-S-dCas9-VP64 | GAGCAGCGTCTTCGAGAGTGAGG | SEQ ID NO: 39 |
| RNPA (VEGFA-A) | | |
| gRNA for PS-S-dCas9-VP64 | GGTGAGTGAGTGTGTGCGTGTGG | SEQ ID NO: 40 |
| RNPB (VEGFA-B) | | |

*indicates phosphorothioated nucleotide.

### P Embodiments

P Embodiment 1. A complex for delivering a gene editing agent to a cell, said complex comprising a gene editing agent covalently bound to a phosphorothioate nucleic acid through a chemical linker.

P Embodiment 2. The complex of P embodiment 1, wherein said gene editing agent comprises a cysteine and said phosphorothioate nucleic acid comprises a thiol moiety covalently bound to said gene editing agent through a disulfide linkage between said cysteine and said thiol moiety.

P Embodiment 3. The complex of P embodiment 1 or 2, wherein said phosphorothioate nucleic acid is bound to the C-terminus of said gene editing agent.

P Embodiment 4. The complex of any one of P embodiments 1-3, wherein said chemical linker is a pH-sensitive linker.

P Embodiment 5. The complex of any one of P embodiments 1, 3 or 4, wherein said chemical linker is a thioester linker.

P Embodiment 6. The complex of any one of P embodiments 1-5, wherein said phosphorothioate nucleic acid is a single stranded nucleic acid.

P Embodiment 7. The complex of any one of P embodiments 1-6, wherein said phosphorothioate nucleic acid is a phosphorothioate deoxyribonucleic acid.

P Embodiment 8. The complex of any one of P embodiments 1-7, wherein said phosphorothioate nucleic acid is about 10, 20, 30, 40, 50, 60, 70, 80, 90, 100 or more nucleic acid residues in length.

P Embodiment 9. The complex of any one of P embodiments 1-8, wherein said phosphorothioate nucleic acid is from about 10 to about 30 nucleic acid residues in length.

P Embodiment 10. The complex of any one of P embodiments 1-9, wherein said phosphorothioate nucleic acid is about 20 nucleic acid residues in length.

P Embodiment 11. The complex of any one of P embodiments 1-10, wherein said gene editing agent is an RNA-guided DNA endonuclease, a transcription activator-like effector nuclease (TALEN), a zinc finger nuclease, or an Argonaut endonuclease.

P Embodiment 12. The complex of P embodiment 11, wherein said RNA-guided DNA endonuclease is Cas9, Cpf1 or a Class II CRISPR endonuclease.

P Embodiment 13. The complex of P embodiment 11 or 12, wherein said RNA-guided DNA endonuclease is nuclease-deficient.

P Embodiment 14. The complex of any one of P embodiments 1-13, further comprising one or more guide RNAs bound to said gene editing agent.

P Embodiment 15. The complex of P embodiment 14, wherein said one or more guide RNAs are complementary to one or more target sequences in said cell.

P Embodiment 16. The complex of P embodiment 15, wherein said one or more target sequence is a STAT-3 target sequence.

P Embodiment 17. The complex of any one of P embodiments 14-16, wherein said guide RNA comprises the sequence of SEQ ID NO: 1.

P Embodiment 18. A complex for delivering a gene editing agent to a cell, said complex comprising:
(i) a double-stranded phosphorothioate oligonucleotide;
(ii) a first gene editing agent covalently bound to a first phosphorothioate nucleic acid through a first chemical linker; and
(ii) a second gene editing agent covalently bound to a second phosphorothioate nucleic acid through a second chemical linker;
wherein at least a portion of said first phosphorothioate nucleic acid and a portion of said second phosphorothioate nucleic acid are complementary to each other and wherein at least a portion of said first phosphorothioate nucleic acid is hybridized to at least a portion of said second phosphorothioate nucleic acid thereby forming said double-stranded phosphorothioate oligonucleotide.

P Embodiment 19. The complex of P embodiment 18, wherein said first gene editing agent and said second gene editing agent are independently an RNA-guided DNA endonuclease, a transcription activator-like effector nuclease (TALEN), a zinc finger nuclease, or an Argonaut endonuclease.

P Embodiment 20. The complex of P embodiment 18 or 19, wherein said first gene editing agent and said second gene editing agent are independently Cas9, Cpf1 or a Class II CRISPR endonuclease.

P Embodiment 21. The complex of P embodiment 18 or 19, wherein said first gene editing agent is a first Cas9 and said second gene editing agent is a second Cas9.

P Embodiment 22. The complex of P embodiment 21, wherein said first gene editing agent and said second gene editing agent are independently nuclease-deficient.

P Embodiment 23. The complex of any one of P embodiments 18-22, wherein said first gene editing agent comprises a first cysteine and said first phosphorothioate nucleic acid comprises a first thiol moiety covalently bound to said first gene editing agent through a disulfide linkage between said first cysteine and said first thiol moiety.

P Embodiment 24. The complex of any one of P embodiments 18-23, wherein said second gene editing agent comprises a second cysteine and said second phosphorothioate nucleic acid comprises a second thiol moiety covalently bound to said second gene editing agent through a disulfide linkage between said second cysteine and said second thiol moiety.

P Embodiment 25. The complex of any one of P embodiments 18-24, wherein said first phosphorothioate nucleic acid is bound to the C-terminus of said first gene editing agent.

P Embodiment 26. The complex of any one of P embodiments 18-25, wherein said second phosphorothioate nucleic acid is bound to the C-terminus of said second gene editing agent.

P Embodiment 27. The complex of any one of P embodiments 18-26, wherein said first chemical linker and said second chemical linker are independently a pH-sensitive linker.

P Embodiment 28. The complex of any one of P embodiments 18-27, wherein said first chemical linker and said second chemical linker are independently a thioester linker.

P Embodiment 29. The complex of any one of P embodiments 18-28, wherein said first phosphorothioate nucleic acid and said second phosphorothioate nucleic acid are independently a phosphorothioate deoxyribonucleic acid or a phosphorothioate ribonucleic acid.

P Embodiment 30. The complex of any one of P embodiments 18-29, wherein said first phosphorothioate nucleic acid and said second phosphorothioate nucleic acid are independently about 10, 20, 30, 40, 50, 60, 70, 80, 90, 100 or more nucleic acid residues in length.

P Embodiment 31. The complex of any one of P embodiments 18-30, wherein said first phosphorothioate nucleic acid and said second phosphorothioate nucleic acid are independently from about 10 to about 30 nucleic acid residues in length.

P Embodiment 32. The complex of any one of P embodiments 18-31, wherein said first phosphorothioate nucleic acid and said second phosphorothioate nucleic acid are independently from about 10 to about 30 nucleic acid residues in length.

P Embodiment 33. The complex of P embodiment 32, wherein said one or more guide RNAs are complementary to one or more target sequences in said cell.

P Embodiment 34. A method of delivering a gene editing agent to a cell, said method comprising contacting a cell with a complex of any one of P embodiments 1-33 thereby delivering said gene editing agent to said cell.

P Embodiment 35. The method of P embodiment 34, wherein said contacting occurs in vitro or in vivo.

P Embodiment 36. The method of P embodiment 34 or 35, wherein said cell is a T cell or a stem cell.

### Embodiments

Embodiment 1. A complex for delivering a gene editing agent to a cell, said complex comprising a gene editing agent covalently bound to a phosphorothioate nucleic acid through a chemical linker.

Embodiment 2. The complex of embodiment 1, wherein said gene editing agent comprises a cysteine and said phosphorothioate nucleic acid comprises a thiol moiety covalently bound to said gene editing agent through a disulfide linkage between said cysteine and said thiol moiety.

Embodiment 3. The complex of embodiment 1 or 2, wherein said phosphorothioate nucleic acid is bound to the C-terminus of said gene editing agent.

Embodiment 4. The complex of any one of embodiments 1-3, wherein said chemical linker is a pH-sensitive linker.

Embodiment 5. The complex of any one of embodiments 1, 3 or 4, wherein said chemical linker is a thioester linker.

Embodiment 6. The complex of any one of embodiments 1-5, wherein said phosphorothioate nucleic acid is a single stranded nucleic acid.

Embodiment 7. The complex of any one of embodiments 1-6, wherein said phosphorothioate nucleic acid is a phosphorothioate deoxyribonucleic acid.

Embodiment 8. The complex of any one of embodiments 1-7, wherein said phosphorothioate nucleic acid is about 10, 20, 30, 40, 50, 60, 70, 80, 90, 100 or more nucleic acid residues in length.

Embodiment 9. The complex of any one of embodiments 1-8, wherein said phosphorothioate nucleic acid is from about 10 to about 30 nucleic acid residues in length.

Embodiment 10. The complex of any one of embodiments 1-9, wherein said phosphorothioate nucleic acid is about 20 nucleic acid residues in length.

Embodiment 11. The complex of any one of embodiments 1-10, wherein said gene editing agent is an RNA-guided DNA endonuclease, a transcription activator-like effector nuclease (TALEN), a zinc finger nuclease, or an Argonaut endonuclease.

Embodiment 12. The complex of embodiment 11, wherein said RNA-guided DNA endonuclease is Cas9, Cpf1 or a Class II CRISPR endonuclease.

Embodiment 13. The complex of embodiment 11 or 12, wherein said RNA-guided DNA endonuclease is nuclease-deficient.

Embodiment 14. The complex of any one of embodiments 1-13, further comprising one or more guide RNA bound to said gene editing agent.

Embodiment 15. The complex of any one of embodiments 1-14, wherein said complex forms part of a cell.

Embodiment 16. The complex of embodiment 15, wherein said cell is a cancer cell or a healthy cell.

Embodiment 17. The complex of embodiment 15 or 16, wherein said cell is a T cell, a chimeric antigen receptor (CAR) T cell, a natural killer (Nk) cell, a macrophage, a neuronal cell or a hematopoietic stem cell.

Embodiment 18. The complex of embodiment 15 or 16, wherein said cell is a pancreatic cancer cell or an ovarian cancer cell.

Embodiment 19. The complex of any one of embodiments 14-19, wherein said one or more guide RNA is complementary to one or more target sequences in said cell.

Embodiment 20. The complex of embodiment 19, wherein said one or more target sequence is a STAT-3 target sequence.

Embodiment 21. The complex of any one of embodiments 14-20, wherein said one or more guide RNA comprises the sequence of SEQ ID NO:1.

Embodiment 22. A complex for delivering a gene editing agent to a cell, said complex comprising:
(i) a double-stranded phosphorothioate oligonucleotide;
(ii) a first gene editing agent covalently bound to a first phosphorothioate nucleic acid through a first chemical linker; and
(ii) a second gene editing agent covalently bound to a second phosphorothioate nucleic acid through a second chemical linker;
wherein at least a portion of said first phosphorothioate nucleic acid and a portion of said second phosphorothioate nucleic acid are complementary to each other and wherein at least a portion of said first phosphorothioate nucleic acid is hybridized to at least a portion of said second phosphorothioate nucleic acid thereby forming said double-stranded phosphorothioate oligonucleotide.

Embodiment 23. The complex of embodiment 22, wherein said first gene editing agent and said second gene editing agent are independently an RNA-guided DNA endonuclease, a transcription activator-like effector nuclease (TALEN), a zinc finger nuclease, or an Argonaut endonuclease.

Embodiment 24. The complex of embodiment 22 or 23, wherein said first gene editing agent and said second gene editing agent are independently Cas9, Cpf1 or a Class II CRISPR endonuclease.

Embodiment 25. The complex of embodiment 22 or 23, wherein said first gene editing agent is a first Cas9 and said second gene editing agent is a second Cas9.

Embodiment 26. The complex of embodiment 25, wherein said first gene editing agent and said second gene editing agent are independently nuclease-deficient.

Embodiment 27. The complex of any one of embodiments 22-26, wherein said first gene editing agent comprises a first cysteine and said first phosphorothioate nucleic acid comprises a first thiol moiety covalently bound to said first gene editing agent through a disulfide linkage between said first cysteine and said first thiol moiety.

Embodiment 28. The complex of any one of embodiments 22-27, wherein said second gene editing agent comprises a second cysteine and said second phosphorothioate nucleic acid comprises a second thiol moiety covalently bound to said second gene editing agent through a disulfide linkage between said second cysteine and said second thiol moiety.

Embodiment 29. The complex of any one of embodiments 22-28, wherein said first phosphorothioate nucleic acid is bound to the C-terminus of said first gene editing agent.

Embodiment 30. The complex of any one of embodiments 22-29, wherein said second phosphorothioate nucleic acid is bound to the C-terminus of said second gene editing agent.

Embodiment 31. The complex of any one of embodiments 22-30, wherein said first chemical linker and said second chemical linker are independently a pH-sensitive linker.

Embodiment 32. The complex of any one of embodiments 22-31, wherein said first chemical linker and said second chemical linker are independently a thioester linker.

Embodiment 33. The complex of any one of embodiments 22-32, wherein said first phosphorothioate nucleic acid and said second phosphorothioate nucleic acid are independently a phosphorothioate deoxyribonucleic acid or a phosphorothioate ribonucleic acid.

Embodiment 34. The complex of any one of embodiments 22-33, wherein said first phosphorothioate nucleic acid and said second phosphorothioate nucleic acid are independently about 10, 20, 30, 40, 50, 60, 70, 80, 90, 100 or more nucleic acid residues in length.

Embodiment 35. The complex of any one of embodiments 22-34, wherein said first phosphorothioate nucleic acid and said second phosphorothioate nucleic acid are independently from about 10 to about 30 nucleic acid residues in length.

Embodiment 36. The complex of any one of embodiments 22-35, further comprising one or more guide RNA bound to said first gene editing agent or said second gene editing agent.

Embodiment 37. The complex of any one of embodiments 22-36, wherein said complex forms part of a cell.

Embodiment 38. The complex of embodiment 37, wherein said cell is a cancer cell or a healthy cell.

Embodiment 39. The complex of embodiment 37 or 38, wherein said cell is a T cell, a chimeric antigen receptor (CAR) T cell, a natural killer (Nk) cell, a macrophage, a neuronal cell or a hematopoietic stem cell.

Embodiment 40. The complex of embodiment 37 or 38, wherein said cell is a pancreatic cancer cell or an ovarian cancer cell.

Embodiment 41. The complex of any one of embodiments 22-40, wherein said one or more guide RNA is complementary to one or more target sequence in said cell.

Embodiment 42. The complex of embodiment 41, wherein said one or more target sequence is a STAT-3 target sequence.

Embodiment 43. The complex of any one of embodiments 36-42, wherein said one or more guide RNA comprises the sequence of SEQ ID NO:1.

Embodiment 44. A complex for delivering a gene editing agent to a cell, said complex comprising:
(i) a double-stranded phosphorothioate oligonucleotide;
(ii) a gene editing agent covalently bound to a first phosphorothioate nucleic acid through a first chemical linker; and
(ii) a targeting agent covalently bound to a second phosphorothioate nucleic acid through a second chemical linker;
wherein at least a portion of said first phosphorothioate nucleic acid and a portion of said second phosphorothioate nucleic acid are complementary to each other; and
wherein at least a portion of said first phosphorothioate nucleic acid is hybridized to at least a portion of said second phosphorothioate nucleic acid thereby forming said double-stranded phosphorothioate oligonucleotide.

Embodiment 45. The complex of embodiment 44, wherein said gene editing agent is an RNA-guided DNA endonuclease, a transcription activator-like effector nuclease (TALEN), a zinc finger nuclease, or an Argonaut endonuclease.

Embodiment 46. The complex of embodiment 44 or 45, wherein said gene editing agent is Cas9, Cpf1 or a Class II CRISPR endonuclease.

Embodiment 47. The complex of any one of embodiments 44-46, wherein said gene editing agent is Cas9.

Embodiment 48. The complex of any one of embodiments 44-47, wherein said targeting agent is a protein or a nucleic acid.

Embodiment 49. The complex of any one of embodiments 44-48, wherein said targeting agent is an antibody.

Embodiment 50. The complex of any one of embodiments 44-49, wherein said targeting agent is a cancer-specific antibody.

Embodiment 51. The complex of any one of embodiments 44-50, wherein said gene editing agent is nuclease-deficient.

Embodiment 52. The complex of any one of embodiments 44-51, wherein said gene editing agent comprises a first cysteine and said first phosphorothioate nucleic acid comprises a first thiol moiety covalently bound to said gene editing agent through a disulfide linkage between said first cysteine and said first thiol moiety.

Embodiment 53. The complex of any one of embodiments 44-52, wherein said targeting agent comprises a second cysteine and said second phosphorothioate nucleic acid comprises a second thiol moiety covalently bound to said targeting agent through a disulfide linkage between said second cysteine and said second thiol moiety.

Embodiment 54. The complex of any one of embodiments 44-53, wherein said first phosphorothioate nucleic acid is bound to the C-terminus of said gene editing agent.

Embodiment 55. The complex of any one of embodiments 44-54, wherein said second phosphorothioate nucleic acid is independently attached to a lysine, arginine, cysteine, or histidine of said targeting agent.

Embodiment 56. The complex of any one of embodiments 44-55, wherein said first chemical linker and said second chemical linker are independently a pH-sensitive linker.

Embodiment 57. The complex of any one of embodiments 44-56, wherein said first chemical linker and said second chemical linker are independently a thioester linker.

Embodiment 58. The complex of any one of embodiments 44-57, wherein said first phosphorothioate nucleic acid and said second phosphorothioate nucleic acid are independently a phosphorothioate deoxyribonucleic acid or a phosphorothioate ribonucleic acid.

Embodiment 59. The complex of any one of embodiments 44-58, wherein said first phosphorothioate nucleic acid and said second phosphorothioate nucleic acid are independently about 10, 20, 30, 40, 50, 60, 70, 80, 90, 100 or more nucleic acid residues in length.

Embodiment 60. The complex of any one of embodiments 44-58, wherein said first phosphorothioate nucleic acid and said second phosphorothioate nucleic acid are independently from about 10 to about 30 nucleic acid residues in length.

Embodiment 61. The complex of any one of embodiments 44-60, further comprising one or more guide RNA bound to said gene editing agent.

Embodiment 62. The complex of any one of embodiments 44-36, wherein said complex forms part of a cell.

Embodiment 63. The complex of embodiment 62, wherein said cell is a cancer cell or a healthy cell.

Embodiment 64. The complex of embodiment 62 or 63, wherein said cell is a T cell, a chimeric antigen receptor (CAR) T cell, a natural killer (Nk) cell, a macrophage, a neuronal cell or a hematopoietic stem cell.

Embodiment 65. The complex of embodiment 62 or 63, wherein said cell is a pancreatic cancer cell or an ovarian cancer cell.

Embodiment 66. The complex of any one of embodiments 44-65, wherein said one or more guide RNA is complementary to one or more target sequence in said cell.

Embodiment 67. The complex of embodiment 66, wherein said one or more target sequence is a STAT-3 target sequence.

Embodiment 68. The complex of any one of embodiments 61-67, wherein said one or more guide RNA comprises the sequence of SEQ ID NO:1.

Embodiment 69. A pharmaceutical composition comprising a complex of any one of embodiments 1-68 and a pharmaceutically acceptable excipient.

Embodiment 70. A method of delivering a gene editing agent to a cell, said method comprising contacting a cell with a complex of any one of embodiments 1-68 thereby delivering said gene editing agent to said cell.

Embodiment 71. The method of embodiment 70, wherein said contacting occurs in vitro or in vivo.

Embodiment 72. The method of embodiment 70 or 71, wherein said cell is a cancer cell or a healthy cell.

Embodiment 73. The method of embodiment 70 or 71, wherein said cell is a T cell, a chimeric antigen receptor (CAR) T cell, a natural killer (Nk) cell, a macrophage, a neuronal cell or a hematopoietic stem cell.

Embodiment 74. The method of embodiment 70 or 71, wherein said cell is a pancreatic cancer cell or an ovarian cancer cell.

1. A complex for delivering a gene editing agent to a cell, said complex comprising a gene editing agent covalently bound to a phosphorothioate nucleic acid through a chemical linker.

2. The complex of claim 1, wherein said gene editing agent comprises a cysteine and said phosphorothioate nucleic acid comprises a thiol moiety covalently bound to said gene editing agent through a disulfide linkage between said cysteine and said thiol moiety.

3. The complex of claim 1, wherein said phosphorothioate nucleic acid is bound to the C-terminus of said gene editing agent.

4. The complex of claim 1, wherein said chemical linker is a pH-sensitive linker.

5. The complex of claim 1, wherein said chemical linker is a thioester linker.

6.-14. (canceled)

15. The complex of claim 1, wherein said complex forms part of a cell.

16. The complex of claim 15, wherein said cell is a cancer cell or a healthy cell.

17. The complex of claim 15, wherein said cell is a T cell, a chimeric antigen receptor (CAR) T cell, a natural killer (Nk) cell, a macrophage, a neuronal cell or a hematopoietic stem cell.

18. (canceled)

19. (canceled)

20. (canceled)

21. (canceled)

22. A complex for delivering a gene editing agent to a cell, said complex comprising:
(i) a double-stranded phosphorothioate oligonucleotide;
(ii) a first gene editing agent covalently bound to a first phosphorothioate nucleic acid through a first chemical linker; and
(iii) a second gene editing agent covalently bound to a second phosphorothioate nucleic acid through a second chemical linker;
wherein at least a portion of said first phosphorothioate nucleic acid and a portion of said second phosphorothioate nucleic acid are complementary to each other and wherein at least a portion of said first phosphorothioate nucleic acid is hybridized to at least a portion of said second phosphorothioate nucleic acid thereby forming said double-stranded phosphorothioate oligonucleotide.

23.-26. (canceled)

27. The complex of claim 22, wherein said first gene editing agent comprises a first cysteine and said first phosphorothioate nucleic acid comprises a first thiol moiety covalently bound to said first gene editing agent through a disulfide linkage between said first cysteine and said first thiol moiety.

28. The complex of claim 22, wherein said second gene editing agent comprises a second cysteine and said second phosphorothioate nucleic acid comprises a second thiol moiety covalently bound to said second gene editing agent through a disulfide linkage between said second cysteine and said second thiol moiety.

29. The complex of claim 22, wherein said first phosphorothioate nucleic acid is bound to the C-terminus of said first gene editing agent.

30. The complex of claim 22, wherein said second phosphorothioate nucleic acid is bound to the C-terminus of said second gene editing agent.

31. The complex of claim 22, wherein said first chemical linker and said second chemical linker are independently a pH-sensitive linker.

32. The complex of claim 22, wherein said first chemical linker and said second chemical linker are independently a thioester linker.

33.-36. (canceled)

37. The complex of claim 22, wherein said complex forms part of a cell.

38. The complex of claim 37, wherein said cell is a cancer cell or a healthy cell.

39. The complex of claim 37, wherein said cell is a T cell, a chimeric antigen receptor (CAR) T cell, a natural killer (Nk) cell, a macrophage, a neuronal cell or a hematopoietic stem cell.

40. (canceled)

41. The complex of claim 22, wherein said one or more guide RNA is complementary to one or more target sequence in said cell.

42. The complex of claim 41, wherein said one or more target sequence is a STAT-3 target sequence, a Programmed cell death protein 1 (PDCD1) target sequence, a Programmed cell death protein 1 (PDCD2) target sequence, a Tet methylcytosine dioxygenase 2 (TET2) target sequence, a PARG Poly (ADP-ribose) glycohydrolase target sequence, a T cell receptor-alpha (TCR-a) target sequence, a T cell receptor-beta (TCR-b) target sequence, a Vascular endothelial growth factor A-alpha (VEGFA-a) target sequence or a Vascular endothelial growth factor A-beta (VEGFA-b) target sequence.

43. (canceled)

44. A complex for delivering a gene editing agent to a cell, said complex comprising:
(i) a double-stranded phosphorothioate oligonucleotide;
(ii) a gene editing agent covalently bound to a first phosphorothioate nucleic acid through a first chemical linker; and
(iii) a targeting agent covalently bound to a second phosphorothioate nucleic acid through a second chemical linker;
wherein at least a portion of said first phosphorothioate nucleic acid and a portion of said second phosphorothioate nucleic acid are complementary to each other; and
wherein at least a portion of said first phosphorothioate nucleic acid is hybridized to at least a portion of said second phosphorothioate nucleic acid thereby forming said double-stranded phosphorothioate oligonucleotide.

45.-51. (canceled)

52. The complex of claim 44, wherein said gene editing agent comprises a first cysteine and said first phosphorothioate nucleic acid comprises a first thiol moiety covalently bound to said gene editing agent through a disulfide linkage between said first cysteine and said first thiol moiety.

53. The complex of claim 44, wherein said targeting agent comprises a second cysteine and said second phosphorothioate nucleic acid comprises a second thiol moiety covalently bound to said targeting agent through a disulfide linkage between said second cysteine and said second thiol moiety.

54. The complex of claim 44, wherein said first phosphorothioate nucleic acid is bound to the C-terminus of said gene editing agent.

55. The complex of claim 44, wherein said second phosphorothioate nucleic acid is independently attached to a lysine, arginine, cysteine, or histidine of said targeting agent.

56. The complex of claim 44, wherein said first chemical linker and said second chemical linker are independently a pH-sensitive linker.

57. The complex of claim 44, wherein said first chemical linker and said second chemical linker are independently a thioester linker.

58.-61. (canceled)

62. The complex of claim 44, wherein said complex forms part of a cell.

63. The complex of claim 62, wherein said cell is a cancer cell or a healthy cell.

64. The complex of claim 62, wherein said cell is a T cell, a chimeric antigen receptor (CAR) T cell, a natural killer (Nk) cell, a macrophage, a neuronal cell or a hematopoietic stem cell.

65.-68. (canceled)

69. A pharmaceutical composition comprising a complex of any one of claims 1, 22 or 44 and a pharmaceutically acceptable excipient.

70. A method of delivering a gene editing agent to a cell, said method comprising contacting a cell with a complex of any one of claims 1, 22 or 44 thereby delivering said gene editing agent to said cell.

71.-74. (canceled)
